(19) United States
(12) Patent Application Publication
(10) Pub. No.: US 2004/0216174 A1
(43) Pub. Date: Oct. 28, 2004

(54) Screening assays for targets and drugs useful in treatment and prevention of lipid metabolism disorders

(76) Inventors: Siegfried Hekimi, Montreal (CA); Yukimasa Shibata, Kobe (JP); Robyn Branicky, Montreal (CA)

Correspondence Address: JONES DAY, 222 EAST 41ST ST, NEW YORK, NY, 10017, US

(21) Appl. No.: 10/800,333

(22) Filed: Mar. 12, 2004

Related U.S. Application Data
(60) Provisional application No. 60/454,925, filed on Mar. 14, 2003.

(51) Int. Cl. A01K 67/033

(52) U.S. Cl. 800/3; 800/8

(57) ABSTRACT

The invention relates to the use of C. elegans as a model for identifying genes that are involved in lipid or lipoprotein metabolism and which can serve as drug targets. The invention also provides a system for screening drugs useful in the treatment and prevention of diseases associated with undesirable or abnormal levels of lipids (e.g., cholesterol) or lipoproteins (e.g., LDL), such as cardiovascular disorders and dyslipidemia.

The present application claims the benefit of U.S. provisional application No. 60/454,925, filed on Mar. 14, 2003, which is incorporated herein by reference in its entirety.

### 1. FIELD OF THE INVENTION

The invention relates to the use of C. elegans as a model for the discovery of potential drug targets. The invention provides a system for screening drugs useful in the treatment and prevention of certain lipid metabolism disorders such as cardiovascular diseases and dyslipidemia.

### 2. BACKGROUND OF THE INVENTION

### 2.1 C. elegans clk1 gene and pleiotropic phenotypes of clk-1 mutants

Mutations in the Caenorhabditis elegans gene clk-1 are highly pleiotropic, affecting the rates of physiological traits that occur over a wide range of timescales (Wong et al., 1995, Genetics 139: 1247-1259). They result in a mean lengthening of the cell cycle of early embryos, embryonic and post-embryonic development, as well as the defecation, swimming, and pharyngeal pumping cycles of adults. clk-1 mutations also affect reproductive features, like the egg-production rate and self-brood size, which are both reduced, and lead to an increased life span.

A number of observations suggest that the phenotypes of clk-1 mutants are the result of an inability to appropriately set the rate of physiological processes (Branicky et al., 2000, Bioessays 22: 48-56 and Wong et al., 1995, Genetics 139: 1247-1259). One example is that many of the features affected by clk-1 mutations are more variable, in addition to being slower on average. For instance, although the average length of embryogenesis of clk-1 mutants is slower than that of the wild type, some clk-1 embryos can develop faster than wild-type embryos, while others take more than two times longer, which suggests that timing is deregulated in the mutants (Wong et al., 1995, Genetics 139: 1247-1259). Also, clk-1 mutant embryos are unable to properly adjust their rate of development in response to changes in temperature. When wild-type embryos are cultured to the 2-cell stage at a particular temperature, and are then transferred to a new temperature, they immediately develop at a rate corresponding to that new temperature. In contrast, when clk-1 mutant embryos are transferred to a new temperature, the rate of development at the new temperature is strongly influenced by the temperature experienced before the shift (Wong et al., 1995, Genetics 139: 1247-1259). This suggests that clk-1 might also be needed to re-set physiological rates in response to changes in temperature. Finally, all of the phenotypes affected in clk-1 mutants can be maternally rescued, that is, homozygous mutant progeny issued from a heterozygous hermaphrodite are phenotypically wild-type. This maternal rescue extends to adulthood, such that all adult behaviors, and even the long life of clk-1 mutants are rescued (Hekimi et al., 1995, Genetics 141: 1351-1364 and Wong et al., 1995, Genetics 139: 1247-1259). Thus, clk-1 is believed to affect a regulatory process that is involved in setting physiological rates in the worm (Branicky et al., 2000, Bioessays 22: 48-56; Felkai et al., 1999, EMBO J 18: 1783-1792; Wong et al., 1995, Genetics 139: 1247-1259). In the presence of maternally supplied clk-1 product, timing can be set appropriately early in development so that homozygous clk-1 mutants can subsequently develop and behave like the wild type.

clk-1 encodes a mitochondrial protein that is highly conserved, structurally and functionally, among eukaryotes (Ewbank et al., 1997, Science 275: 980-983; Jonassen et al., 1996, Arch Biochem Biophys 330: 285-289; Proft et al.,1995, EMBO J 14: 6116-6126; Vajo et al., 1999, Mamm Genome 10: 1000-1004) and encodes a putative hydroxylase (Stenmark et al., 2001, J Biol Chem 276:33297-300) that is required for the biosynthesis of ubiquinone, (UQ, also called coenzyme Q, CoQ), a prenylated benzoquinone lipid that functions as a transporter of electrons in complexes II and III of the respiratory chain. Mutants of the yeast homologue of clk-1, coq-7, do not produce UQ, and therefore cannot grow on non-fermentable carbon sources (Marbois and Clarke, 1996, J Biol Chem 271: 2995-3004). Mitochondria isolated from clk-1 mutants also do not contain detectable levels of UQ but instead accumulate the UQ biosynthetic intermediate, demethoxyubiquinone (DMQ) (Miyadera et al., 2001, J Biol Chem 276: 7713-7716). In clk-1 mutants this compound functions as an electron carrier, such that the mitochondria can maintain respiration despite the complete absence of UQ (Felkai et al., 1999, EMBO J 18: 1783-1792 and Miyadera et al., 2001, J Biol Chem 276: 7713-7716). However, DMQ cannot entirely substitute for UQ as clk-1 mutants cannot complete development when they are fed E. coli strains that do not produce UQ (Jonassen et al., 2001, PNAS 98: 421-426).

It is, however, not clear how the absence of UQ relates to the other mutant phenotypes as there is no correlation between this biochemical phenotype and the severity of the overall phenotype. Indeed, the quinone phenotype is identical for all three known clk-1 alleles (e2519, qm30 and qm51): UQ is totally absent from mitochondria in all three cases, and all three accumulate the same amount of DMQ. Yet, most of the features affected in clk-1 mutants are slowed down much more severely in the putative null alleles qm30 and qm51, than they are in the partial loss of function allele e2519 (Felkai et al., 1999, EMBO J 18: 1783-1792 and Wong et al., 1995, Genetics 139: 1247-1259). Also, by various measures of energy metabolism in intact worms, clk-1 mutants have been shown to have metabolic capacities and ATP levels comparable to the wild type (Braeckman et al., 1999, Curr Biol 9: 493-496.). Together, these observations suggest that much of the phenotype of clk-1 mutants may not be the direct consequence of an absence of UQ in mitochondria or a decreased level of energy production.

One of the features affected in clk-1 mutants is the defecation cycle. In C. elegans, defecation is effected by a stereotyped Defecation Motor Program (DMP). The DMP consists of three distinct steps: the posterior body muscle contraction (pBoc), the anterior body muscle contraction (aBoc), and the expulsion (Exp), which consists of the enteric muscle contractions (EMC) (Thomas et al., 1990, Genetics 124: 855-872.). In the presence of adequate food, the defecation cycle period of 56 seconds is regular in single animals over time and among animals, with a standard deviation of only a few seconds. In addition to its tight periodicity, the defecation cycle has other properties that suggest that it might be controlled by an endogenous “clock”. For example, the phase of the cycle can be reset by lightly touching the animal, and the rhythm is maintained even in the absence of expression of the DMP (Liu and Thomas, 1994, J Neurosci 14: 1953-1962).

The periodicity of the defecation cycle can be altered by mutations in at least 13 genes (Dec phenotype). These mutations fall into two major classes: short Dec (Dec-s), for mutations that decrease the cycle length, and long Dec (Dec-L), for mutations that increase the cycle length (Iwasaki et al., 1995, PNAS 92: 10317-10321). The molecular identification of the Dec-L gene, dec-4 (lef-1/itr-1), as the inositol triphosphate receptor (IP3 receptor), a protein involved in regulating intracellular calcium levels, suggests that calcium oscillations contribute to the regulation of the rhythm. Indeed, Dal Santo et al. (1999, Cell 98: 757-767) showed that calcium levels peak in the intestine just prior to the first muscle contraction of the DMP, and that expression of the EP3 receptor in the intestine was sufficient for normal rhythm generation. Although it is not yet clear how the different Dec genes might be interacting to regulate the defecation cycle, the molecular characterization of two other genes, flr-1 and unc-43/dec-8, also support roles for calcium and the intestine in rhythm regulation. flr-1 mutants, originally identified on the basis of their resistance to fluoride (Katsura et al., 1994, Genetics 136: 145-154), have, among other defecation phenotypes, a very short defecation cycle length (Iwasaki et al., 1995, PNAS 92: 10317-10321). flr-1 encodes an ion channel of the degenerin/epithelial sodium channel superfamily, which is expressed only in the intestine from embryos to adults (Take-Uchi et al., 1998, PNAS 95: 11775-11780). Mutations in unc-43 result in multiple behavioral defects including defecation phenotypes (Liu and Thomas, 1994, J Neurosci 14: 1953-1962 and Reiner et al., 1999, Nature 402: 199-203). Loss-of-function mutations result in an increased frequency of defecation, usually the result of a repetition of the DMP ˜13 sec after the initiation of the primary motor program, whereas the gain of function mutation results in a decreased frequency of defecation. unc-43 encodes the C. elegans CaM Kinase II, which is widely expressed in neurons, muscles, and the intestine (Reiner et al., 1999, Nature 402: 199-203).

### 2.2 Lipid Metabolism

Lipids present in the diet must be absorbed and transported in the blood. The metabolism of lipids involves the interaction of lipids, apoproteins, lipoproteins, bile acids, and enzymes. For a review, see Brown & Goldstein, In, The Pharmacological Basis Of Therapeutics, 8th Ed., Goodman & Gilman, Pergamon Press, NY, 1990, Ch. 36, pp. 874-896; and Fuchs, Am. J. Physiol. Gastrointest. Liver Physiol. 284:G551-557.

Lipoproteins are micelle-like assemblies found in plasma which contain varying proportions of different types of lipids and apoproteins. There are five main classes of plasma lipoproteins, in order of increasing density, chylomicrons, very low density lipoproteins (VLDL), intermediate-density lipoproteins (IDL), low density lipoproteins (LDL), and high density lipoproteins (HDL). Although many types of lipid are found associated with each lipoprotein class, each class transports predominantly one type of lipid: triacylglycerols described above are transported in chylomicrons, VLDL, and IDL; while phospholipids and cholesterol esters are transported in HDL and LDL respectively. The apoproteins are noncovalently bound to the surface of lipoproteins and act as binding sites and enzyme cofactors in the metabolism of the various particles. The major apolipoproteins are apoA-I, A-II, A-IV, B-100, B-48, C-I, C-II, C-III, D, and E. ApoB-100 is present in VLDL, IDL and LDL, whereas apoE is present in chylomicron remnants, VLDL and IDL. High levels of circulating LDL and beta-VLDL in blood in particular have been associated with increased risk of cardiovascular heart disease.

Chylomicrons are formed in the intestine from absorbed lipids and apoproteins generated by the intestinal epithelium. They are large particles (80-500 nm) that are less dense than water. Chylomicrons are formed in the intestinal epithelial cells and then pass out off the cells into the tissue fluid. From there they are collected into the central lacteals of the intestinal villi. The lymphatic system carries these large lipoproteins to the general circulation. Chylomicrons transport dietary fats from the intestine to adipose tissue and the liver.

The majority of VLDL are derived from the liver and represent the transport mechanisms for triacylglycerol from the liver to other tissues. The mechanism of manufacture and release is remarkably similar to the particulate secretion of chylomicrons in the intestine. Except for the mammary gland, the liver and intestine are the only tissues that secrete particulate lipid. Particulate lipid is unable to pass through capillary walls without prior hydrolysis and therefore is relegated to the lymphatic system. VLDL deliver endogenously synthesized fats to adipose tissue.

Both chylomicrons and VLDL (30-100 nm) particles are metabolized and cleared from the blood rapidly. Adipose tissue, heart and muscle do most of the metabolism. This is accomplished via the action of the enzyme lipoprotein lipase present in blood vessels and tissues. The lipoprotein complex becomes bound to the walls of blood vessels where the enzyme hydrolyzes triacylglycerol to free fatty acids and glycerol. Some of the free fatty acids are released into the blood but most are transported into the tissues. The resulting chylomicron remnants are much smaller and are enriched in cholesterol and cholesterol esters. These remnants are taken up by the liver by a receptor mediated mechanism.

LDL (25-30 nm) is formed from VLDL and perhaps from chylomicrons. In normal cells, LDL is internalized, cholesterol esters are hydrolyzed, the protein is broken down in lysozomes, and cellular cholesterol synthesis is repressed. The number of LDL binding sites on a cell membrane is regulated by cellular need for cholesterol. Half of the LDL is metabolized in the liver. Factors that increase the synthesis of triacylglycerol and secretion of VLDL by the liver include high carbohydrate diets, ethanol ingestion, high concentrations of insulin, and low concentration of glucagon. VLDL and LDL are atherogenic lipoproteins. ApoB-100 and apoE are ligands for the LDL receptor.

The liver and intestine synthesize and secrete the smallest (7.5-10 nm), most soluble and protein rich lipoproteins, HDL, but the intestinal sources lack a protein that is added later from liver sources. HDL contains cholesterol esters at its core surrounded by phospholipids and protein. Plasma HDL concentrations are inversely related to the incidence of coronary artery disease. HDL is thought to act as a cholesterol scavenger carrying surplus cholesterol from the tissues to the liver. HDL removes cholesterol from peripheral tissues and prevents lipid accumulation in arterial walls.

Cholesterol is the metabolic precursor of steroid hormones and bile acids as well as an essential constituent of cell membranes. In man and other animals, cholesterol is ingested in the diet and also synthesized by the liver and other tissues. Dietary cholesterol absorption, endogenous cholesterol synthesis and biliary cholesterol secretion regulate whole body cholesterol balance. Because elevated plasma cholesterol level is a risk factor for atherosclerosis, and most of the cholesterol in the body is disposed of via the biliary system, the enterohepatic circulation and regulation of bile acid synthesis and transport is a very important part of lipid metabolism. In the liver, cholesterol is converted to 7-hydroxycholesterol and then to cholic acid and chenodeoxycholic acid. These bile acids are reabsorbed via intestine and delivered back to the liver.

Hepatic lipase and lipoprotein lipase are multifunctional proteins which mediate the binding, uptake, catabolism, and remodeling of lipoproteins and phospholipids. Lipoprotein lipase and hepatic lipase function while bound to the luminal surface of endothelial cells in peripheral tissues and the liver respectively. Both enzymes participate in reverse cholesterol transport, which is the movement of cholesterol from peripheral tissues to the liver either for excretion from the body or for recycling. Genetic defects in both hepatic lipase and lipoprotein lipase are known to be the cause of familial disorders of lipoprotein metabolism. Defects in the metabolism of lipoproteins result in serious metabolism disorders, including hypercholesterolemia, hyperlipidemia, and atherosclerosis.

Atherosclerosis is a complex, polygenic disorder which is defined in histological terms by deposits (lipid or fibrolipid plaques) of lipids and of other blood derivatives in blood vessel walls, especially the large arteries (aorta, coronary arteries, carotid). These plaques, which are more or less calcified according to the degree of progression of the atherosclerotic process, may be coupled with lesions and are associated with the accumulation in the vessels of fatty deposits consisting essentially of cholesterol esters. These plaques are accompanied by a thickening of the vessel wall, hypertrophy of the smooth muscle, appearance of foam cells (lipid-laden cells resulting from uncontrolled uptake of cholesterol by recruited macrophages) and accumulation of fibrous tissue. The atheromatous plaque protrudes markedly from the wall causing vascular occlusions by atheroma, thrombosis or embolism, which occur in those patients who are most affected. These lesions can lead to serious cardiovascular pathologies such as infarction, sudden death, cardiac insufficiency, and stroke.

Despite the understanding that has evolved regarding the role of various enzymes and apoproteins in lipid homeostasis, there nevertheless is a need to identify additional genes coding for proteins that participates in and/or regulate lipid metabolism.

### 2.3 Current Treatments for Lipid Metabolism-Related Disorders

The standard treatment modalities include dietary therapy, physical exercise and drug therapy. Currently the drugs recommended for lowering serum cholesterol and triglycerides can be classified into several classes. However, each has its own drawbacks and limitations in terms of efficacy, side-effects and qualifying patient population.

Bile-acid-binding resins are a class of drugs that interrupt the recycling of bile acids from the intestine to the liver; e.g., cholestyramine (Questran Light™., Bristol-Myers Squibb), and colestipol hydrochloride (Colestid™., The Upjohn company). The use of such resins, however, at best only lowers serum cholesterol levels by about 20%, and is associated with gastrointestinal side-effects, including constipation and certain vitamin deficiencies.

The statins are cholesterol lowering agents that block cholesterol synthesis by inhibiting AMGCoA reductase—the key enzyme involved in the cholesterol biosynthetic pathway. The statins, e.g., lovastatin (Mevacor™, Merck & Co., Inc.) and pravastatin (Pravachol™, Bristol-Myers Squibb Co.) are sometimes used in combination with bile-acid-binding resins. The statins significantly reduce serum cholesterol and LDL-serum levels, and slow progression of coronary atherosclerosis. However, serum HDL cholesterol levels are only slightly increased. The mechanism of the LDL lowering effect may involve both reduction of VLDL concentration and induction of cellular expression of LDL-receptor, leading to reduced production and/or increased catabolism of LDLs. Side effects, including liver and kidney dysfunction are associated with the use of these drugs (Physicians Desk Reference, Medical Economics Co., Inc., Montvale, N.J. 1997).

Niacin, or nicotinic acid, is a water soluble vitamin B-complex used as a dietary supplement and antihyperlipidemic agent. Niacin diminishes production of VLDL and is effective at lowering LDL. It is used in combination with bile-acid binding resins. Niacin can increase HDL when used at adequate doses, however, its usefulness is limited by serious side effects when used at such doses.

Fibrates are a class of lipid-lowering drugs used to treat various forms of hyperlipidemia, (i.e., elevated serum triglycerides) which may also be associated with hypercholesterolemia. For example, clofibrate (Atromid-S™, Wyeth-Ayerest Laboratories) is an antilipidemic agent which acts (via an unknown mechanism) to lower serum triglycerides by reducing the VLDL fraction. Although serum cholesterol may be reduced in certain patient subpopulations, the biochemical response to the drug is variable, and is not always possible to predict which patients will obtain favorable results. Serious side-effects are associated with the use of fibrates including toxicity such as malignancy, (especially gastrointestinal cancer), gallbladder disease and an increased incidence in non-coronary mortality.

Thus, there is a need to develop safer drugs that are efficacious in lowering serum cholesterol, lowering LDL serum levels, preventing coronary heart disease, and/or treating existing disorder.

### 3. SUMMARY OF THE INVENTION

The invention relates to the use of C. elegans as a model for the discovery of genes involved in lipid metabolism, in particular, the modulation of lipid and/or lipoprotein levels. Such genes, termed Modulators Of Lipids and Lipoproteins (or MOLLs), can serve as targets for drug discovery or can be used in screening assays to identify additional MOLLs.

one embodiment, the invention encompasses a method for selecting nematodes having modulated level of a lipid or lipoprotein comprising: treating test nematodes to modulate the level of a lipid or a lipoprotein; identifying test nematodes that manifest/exhibit a phenotype that is modified as compared to the phenotype of the test nematodes of step (a) that has not been treated, and correlating a modified phenotype with a modulated level of the lipid or lipoprotein in the test nematodes. Examples of phenotypes that can be used include (i) length of defecation cycle; (ii) rate of germline development relative to rate of soma development; (iii) rate of embryonic development; and/or (iv) rate of post-embryonic development.

In another embodiment, the invention encompasses a method for isolating a gene that modulates the level of a lipid or lipoprotein in nematodes comprising: subjecting nematodes that comprise at least one mutation in the clk-1 gene to mutagenesis to produce test nematodes; identifying test nematodes that manifest a phenotype that is modified as compared to the phenotype of the nematodes of step (a) not subjected to mutagenesis, and correlating the modified phenotype with a modulated level of the lipid or lipoprotein in the test nematodes, wherein the phenotype can be (i) length of defecation cycle; (ii) rate of germline development relative to rate of soma development; (iii) rate of embryonic development; and/or (iv) rate of post-embryonic development; and cloning the gene by techniques well known in the art that was mutated in the test nematodes.

In another embodiment, the invention encompasses a method for identifying a gene that modulates the level of a lipid or lipoprotein in nematodes comprising contacting test nematodes that comprise at least one mutation in the clk-1 gene with a nucleic acid that reduces specifically the level of expression of a nematode gene; and correlating a modified phenotype with a change in the level of lipid or lipoprotein in the test nematode, wherein a modification of a phenotype relative to the phenotype of test nematodes not contacted with said nucleic acid indicates that the nematode gene modulates the level of the lipid or lipoprotein in nematodes, said phenotype being any of the following (i) length of defecation cycle; (ii) rate of germline development relative to rate of soma development; (iii) rate of embryonic development; and/or (iv) rate of post-embryonic development.

In another embodiment, the invention encompasses a method of screening for a compound that modulates the level of a lipid or lipoprotein in a nematode comprising: contacting a compound with test nematodes; comparing a phenotype of the test nematodes with the phenotype of nematodes not contacted with the compound, whereby a difference in the phenotypes identifies the compound. The modification of phenotype correlates with a modulated level of a lipid or lipoprotein, the phenotype being selected from the group consisting of (i) length of defecation cycle; (ii) rate of germline development relative to rate of soma development; (iii) rate of embryonic development; and (iv) rate of post-embryonic development.

In various embodiments of the methods, the phenotype that is modified is (i) a decreased length of defecation cycle; (ii) an increased rate of germline development relative to rate of soma development; (iii) an increased rate of embryonic development; or (iv) an increased rate of post-embryonic development.

Examples of such lipids in nematodes that may also be present in mammals include but is not limited to cholesterol, fatty acids, sterols, and ubiquinone. Examples of such lipoproteins in nematodes include, but are not limited to, LDL-like lipoprotein, which contain homologs of human apolipoproteins including the ApoBs, the vitellogenins, and lipoproteins containing fragments of ApoB-like sequences, as well as other lipid-containing particles that comprise such proteins.

The present invention also relates to nucleotide sequences of MOLL genes, particularly dsc-3 and dsc-4, and amino acid sequences of their encoded proteins, as well fragments, derivatives and analogs which are functionally active, i.e., they are capable of displaying one or more known functional activities associated with a full-length wild-type MOLL protein. Such functional activities include but are not limited to antigenicity, immunogenicity, and biological activity (e.g., binding of lipids and apolipoproteins, modulation of cholesterol, LDL and/or ROS levels). In one embodiment, the invention encompasses an isolated MOLL nucleic acid molecule that comprises a nucleotide sequence which is at least 90% identical to the nucleotide sequence of SEQ ID NO:1 or 7; that hybridizes with a nucleic acid probe consisting of the nucleotide sequence of SEQ ID NO:1 or 7, or a complement thereof under stringent conditions; or that comprises a nucleic acid molecule that encodes a polypeptide comprising the amino acid sequence of SEQ ID NO:2 or 8. Complements, fragments and variants of isolated MOLL nucleic acid molecules are also encompassed.

In another embodiment, the invention encompasses an isolated MOLL polypeptide, particularly DSC-3 and DSC-4. In a specific embodiment, the invention encompasses a polypeptide comprising a portion of the amino acid sequence of SEQ ID NO:2 or 8; a naturally occurring allelic variant of SEQ ID NO:2 or 8, and a variant that is at least 90% identical to SEQ ID NO:2 or 8.

Methods of production of the MOLL proteins, derivatives and analogs, e.g., by recombinant means, are also provided. Also encompassed are cells and nematodes containing recombinant MOLL nucleic acids and/or recombinant MOLL polypeptides.

The invention also provides for prophylactic and therapeutic treatment of disorders characterized by undesirable or abnormal levels of lipids, lipoproteins, and/or ROS. Such methods comprise administering to a subject in need thereof an effective amount of an agent of the invention that alters lipids (including sterols, such as cholesterol), lipoproteins (such as LDL) and/or ROS levels such that the pathological phenotype is ameliorated. Agents of the invention encompass compositions capable of modulating the expression level or activity of clk-1 and/or MOLL proteins and nucleic acids as well as MOLL proteins and analogs and derivatives (including fragments) thereof, antibodies thereto; nucleic acids encoding the MOLL proteins, analogs, or derivatives; and MOLL antisense nucleic acids.

### 4. DESCRIPTION OF THE FIGURES

FIGS. 1A-1E: The dsc-4(qm182) mutation and reduced cholesterol intake suppress the slowed germline development of clk-1 mutants. (A) Time course analysis of egg-laying rate. Worms were synchronized at the adult molt (Time 0) and the egg-laying rate (number of eggs laid per hour) was measured at 24 h intervals (n≧30). The genotypes were as follows: Wild type (N2); dsc-4(qm182); clk-1(qm30); and clk-1(qm30)/dsc-4(qm182). Wild-type animals reached peak egg laying between 24 and 48 hours whereas clk-1 mutants reached peak egg laying at 72 hours. The dsc-4(qm182) mutation suppressed as demonstrated by clk-1/dsc-4 double mutants reaching peak egg laying by 48 hours. The dsc-4 single mutants reached peak egg laying by 24 hours, which was slightly earlier than in the wild type. (B) Brood size. The average number of progeny produced by more than 10 animals of each genotype is shown. The error bars represent the standard deviations (SD). The mean brood sizes ± the SD of wild type=315.0±30.4, dsc-4(qm182)=210.9±31.8, clk-1(qm30)=206.8±52.4, and clk-1(qm30)/dsc-4(qm182)=233.8±33.7. (C) Germline development at 6 hours after the adult molt. The percentage of germlines at each of four different developmental stages is shown for each genotype (n≧30). The stages of development of the anterior (A) and posterior (P) gonad arms are presented separately. All wild-type animals were in the process of oogenesis and about half of them had fertilized eggs. In contrast, less than half of the clk-1(qm30) mutants were in the process of oogenesis. The development of the posterior germline was more advanced than the anterior. The dsc-4(qm182) mutation suppressed the slower germline development of clk-1 mutants, but it cannot overcome the difference between the anterior and posterior gonad. (D) Germline development at 1.5 hours after the adult molt. The percentage of germlines at each of four different developmental stages is shown for each genotype on plates with and without cholesterol supplementation (n≧30 and n≧19, respectively). Most wild-type animals, which start primary spermatocyte formation at the late L4 stage, had finished primary spermatocyte formation. In contrast, half of the clk-1(qm30) mutants had not yet started primary spermatocyte formation. The dsc-4(qm182) mutation suppressed the slower germline development of clk-1(qm30) mutants as most of clk-1(qm30)/dsc-4(qm182) double mutants had finished primary spermatocyte formation. (E) Duration of postembryonic development. The percentage of worms that had reached adulthood during each time interval is shown (n≧50). Wild-type animals had all reached adulthood by 51 hours after hatching. clk-1(qm30) mutants had all reached adulthood by 81 hours after hatching, as did clk-1(qm30)/dsc-4(qm182) double mutants, indicating that dsc-4 does not suppress the slow post-embryonic development of clk-1 mutants.

FIGS. 2A-2G: Effects of clk-1 and dsc-4 on germline development and pattern of expression of dsc-4. (A) Schematic representation of the proximal portion of the germline in late L4 and young adult hermaphrodites. The gonad normally consists of 2 U-shaped arms (anterior and posterior) that join the centrally located uterus, and are fused to the vulva. The distal-proximal axis is relative to the vulva, the proximal opening of the gonad to the exterior. (B)-(D) The proximal end of the posterior germline at 6 hours after the adult molt. Left is anterior and top is dorsal. Asterisks indicate the nucleus of the most proximal oocyte. Arrows indicate the proximal end of the germline. The letter “e” indicates fertilized egg. (B) The wild type had oocytes at the proximal end of the germline. (D) clk-1(qm30) mutants were still undergoing spermatogenesis (dotted line). Primary spermatocytes were also observed near the proximal end of the germline (solid line). (C) dsc-4(qm182) single mutants and (E) clk-1(qm30)/dsc-4(qm182) double mutants had oocytes at the proximal end of germline as well as fertilized eggs in the uterus. (F)-(G) GFP fluorescence derived from a dsc-4::GFP transcriptional fusion reporter gene. The outline of the animals and embryos are indicated by the dotted lines. The bar indicates 10 μm. (F) GFP fluorescence observed in the intestine of a comma stage embryo. (G) Posterior end of a late larval worm. GFP fluorescence was observed in the intestine up to and during adulthood.

FIGS. 3A-3C: The structure and homologies of the dsc-4 gene and protein product. (A) The primary structure of DSC-4 polypeptide (SEQ ID NO:2) aligned with the zebrafish, mouse and human MTP. Identical residues are cross-hatched and residues that have >75% and >50% similarity are in black and in light grey, respectively. The asterisks indicate the mutation sites at which mutations were found in dsc-4(qm182). There was a C→T transition at position 354 of the gene results in a serine to phenylalanine substitution at position 62 of the protein and a G→A transition at position 605 of the gene results in an alanine to threonine substitution at position 146 of the protein. The signal sequence is not shown. Domains of DSC-4 include: the apoB binding domain (amino acid residues 19-295), the apoB and PDI binding domain (amino acid residues 296-609), and the lipid binding domain (amino acid residues 610-890). (B) The genomic structure of dsc-4 gene. The filled-in and open boxes correspond to non-coding and coding regions, respectively. Sequencing of a cDNA as well as a PCR product amplified from a first-strand cDNA library revealed that the dsc-4 message is SL1 trans-spliced and contains 11 exons. (C) Schematic representation of the dsc-4 protein. Cross-hatched boxes represent the signal sequence, apoB binding domain, apoB and PDI binding domain and lipid binding and transfer domain.

FIG. 4: Sequences of the dsc-4 cDNA (SEQ ID NO:1) and protein (SEQ ID NO:2). indicates a predicted cleavage site by signal sequence. # indicates sites mutated in dsc-4 (qm182) mutant identified in the suppressor screen. The C to T mutation at nucleotide position 354 results in a serine to phenylalanine substitution at amino acid position 62 and the G to A mutation at nucleotide position 605 results in an alanine to threonine substitution at amino acid position 146. The underlined residues correspond to the apoB binding domain, the dotted underlined residues correspond to the apoB and PDI binding domain, and the double underlined residues correspond to the lipid binding domain. The sequences of the 5′-and 3′-UTRs have also been included. The dsc-4 DNA sequence shown comprises 11 exons which can be found following a 5′ untranslated region at nucleotide bases 1-169. The nucleotide positions of the exons within the dsc-4 coding sequence are as follows: exon 1: 170-215, exon 2: 216-472, exon 3: 473-819, exon 4: 820-924, exon 5: 925-1084, exon 6: 1085-1251, exon 7: 1252-1543, exon 8: 1544-1738, exon 9: 1739-2182. exon 10: 2183-2529, and exon 11: 2530-2848.

FIGS. 5A-5F: RNA interference against vit-3, -4, and -5 suppressed the slow germline development of clk-1 mutants. (A) A comparison of the identity between vit genes. The percentages of identical nucleotides between vit gene sequences are indicated. vit-3, -4 and -5 are virtually identical to each other and show about 66% and 40% homology with vit-2 and -6 respectively. vit-2 has 42% identity with vit-6. For (B)-(E), worms were synchronized at the adult molt (Time 0) and the egg-laying rate (number of eggs laid per hour) was measured at 24 hour intervals (n≧20). (B) The effect of vit RNAi on egg-laying rate. The egg-laying rate of clk-1 mutants at 24 hours after the molt to adulthood is shown (n≧20). Error bars indicate SD. vit-5 RNAi caused weaker suppression than dsc-4 RNAi while vit-2 RNAi and vit-6 RNAi had virtually no effect by this measure. (C) The effect of vit-5 RNAi on egg-laying rate. A time course experiment was performed with the wild type, clk-1(qm30) and clk-1(qm30)/dsc-4(qm182) mutants treated with vit-5 RNAi (n≧27). vit-5 RNAi affected the egg-laying rate only in the clk-1(qm30) background. That the effect of vit-5 RNAi was not additive to that of the dsc-4 mutation. (D) The effect of vit-5 RNAi on germline development. The percentage of animals with eggs in their uteri at each time point is indicated (n≧30). The germline development of clk-1(qm30) was accelerated by vit-5 RNAi, which resulted in the appearance of the first animals with fertilized eggs 9 hours prior to the controls. Furthermore, all vit-5 RNAi treated animals had eggs 15 hours before all the controls did. clk-1 single mutant (E) and clk-1/vit-5 RNAi double mutant (F) animals at 24 hours after the adult molt. Bar indicates 10μm. clk-1(qm30) mutants on the control plates had an average of 0.9±1.6 fertilized eggs in their uteri (n=28), while clk-1/vit-5 RNAi animals had 10.3±1.9 eggs (n=20). A 3X magnified image of the uterus is shown in the bottom right. Arrowheads point to fertilized eggs.

FIGS. 6A-6C: SOD-1 RNAi suppressed the slow germline development of clk-1 mutants. Worms were synchronized at the adult molt (Time 0) and the egg-laying rate (number of eggs laid per hour) was measured at 24 hour intervals. The average egg-laying rate of 13 to 23 animals is shown for each genotype. (A) Effects of RNAi against SOD-1, -2, -3, -4 on the egg-laying rate of the wild type. More than 22 animals were examined for each RNAi treatment. RNAi against SOD-1, -2, -3 and -4 had no effect on the egg-laying rate of wild-type animals. (B) Effect of RNAi against SOD-1, -2, -3, -4 on the egg-laying rate of clk-1 mutants. More than 19 animals were examined for each RNAi treatment. SOD-1 RNAi suppressed the delayed egg-laying of clk-1 mutants. clk-1(qm30) mutants reached peak of egg-laying at 72 hours after the adult molt on the control plate, whereas clk-1(qm30)/SOD-1 RNAi double mutants reached peak of egg-laying at 48 hours. RNAi against SOD-2 and -3 decreased the number of eggs laid per hour but did not significantly affect the time-course of egg-laying, while RNAi against SOD-4 had no effect on clk-1 mutants. (C) Effect of RNAi against SOD-1 on the egg-laying rate of clk-1(qm30)/dsc-4(qm182) double mutants. More than 23 animals were examined for each condition. RNAi against SOD-1 had no effect in either the dsc-4(qm182) or clk-1(qm30)/dsc-4(qm182) mutant backgrounds.

FIGS. 7A-7B: A model of the functional relationships between gene products and processes involved in lipoprotein oxidation in worms and vertebrates. (A) A model for the regulation of C. elegans germline development by lipoprotein. The results described in Sections 7 and 8 indicate that germline development is stimulated by oxidized LDL-like lipoproteins and inhibited by native LDL-like lipoprotein. Activities and processes that favor the production of native lipoproteins are in light grey, and those that favor oxidation are in dark grey. DSC-4 polypeptide, the worm homologue of MTP, is required for the secretion of LDL-like lipoprotein. clk-1 polypeptide is required for the biosynthesis of ubiquinone (UQ), a redox-active lipid that is a major source of ROS, but is also an antioxidant. In clk-1 mutants, UQ is not made but a biosynthetic precursor, demethoxyubiquinone (DMQ), accumulates. DMQ is less prone to ROS production and may also be a better antioxidant. Thus, the LDL-like lipoproteins are less oxidized in clk-1 mutants. On the other hand, RNAi against SOD-1, which encodes the cytosolic superoxide dismutase, leads to an increase in cytoplasmic ROS. As the LDL-like lipoproteins are oxidized by ROS, this counteracts the reduction in oxidation caused by the presence of DMQ, restoring the wild-type ratio of native to oxidized LDL-like lipoproteins, thereby stimulating the development of the germline. The secretion of LDL-like lipoproteins is reduced by mutations in dsc-4, by decreasing cholesterol intake, and by decreasing the production of the VIT proteins, the worm homologues of apoB. These conditions are expected to severely affect both native and oxidized LDL-like lipoproteins, thus restoring the synchronization of soma and germline by removing both inhibitory and stimulatory inputs. (B) A comparison of the elements involved in the secretion and oxidation of lipoproteins in vertebrates and worms. The structural elements involved in the production and oxidation of LDL-like lipoproteins exist in worms and are functionally related in a way similar to that observed in vertebrates. DSC-4 is the worm homologue of MTP while vit-3, vit-4 and vit-5 are the worm homologues of apoB. In worms, as in vertebrates, reduction of cholesterol intake reduces LDL-like lipoprotein secretion and that ROS lead to their oxidation. Oxidized lipoprotein have specific biological effects. In vertebrates, one of the best characterized effects is the initiation of the processes that lead to atherosclerosis. In worms, an effect on the development of the germline was identified. MTP=Microsomal Triglyceride Transfer Protein; apoB=apolipoprotein B; LDL=Low Density Lipoprotein; VIT=Vitellogenin; ROS=Reactive Oxygen Species.

FIGS. 8A-8B: K02D7.4 rescued both the suppression of slow defecation and slow germline development of clk-1(qm30) mutants conferred by dsc-4(qm182). qmEx254 and qmEx251 are two different extrachromosomal arrays containing the K02D7.4 gene. (A) Defecation. The dsc-4(qm182) mutation suppresses the slow defecation of clk-1(qm30) mutants at 20 and 25° C. Both extrachromosomal arrays rescued this suppressing effect. Each bar represents the mean defecation cycle length of >15 animals scored for 5 consecutive defecation cycles at either 20 or 25° C.; the error bars represent the standard deviations of the means. (B) Germline development. The dsc-4(qm182) mutation suppressed the delayed germline development of clk-1 mutants, which resulted in an increased rate of egg laying shortly after the adult molt. Both extrachromosomal arrays rescued this effect. Each bar represents the mean egglaying rate (number of eggs laid per hour) of >30 animals at 24 hours after the 12 adult molt; the error bars represent the standard deviations. That wild-type copies of K02D7.4 rescued the phenotypes caused by the dsc-4(qm182) mutation indicates that dsc-4 corresponds to K02D7.4.

FIG. 9: The nucleotide sequence of the dsc-3 transcript (SEQ ID NO:7). The 22 exons of dsc-3 were identified at the following nucleotide base pairs: exon 1: 1-110, exon 2: 111-224, exon 3: 225-323, exon 4: 324-458, exon 5: 459-613, exon 6: 614-991, exon 7: 992-1272, exon 8: 1273-1341, exon 9: 1342-1764, exon 10: 1765-1890, exon 11: 1891-2088, exon 12: 2089-2232, exon 13: 2233-2320, exon 14: 2321-2469, exon 15: 2470-2620, exon 16: 2621-3014, exon 17: 3015-3147, exon 18: 3148-3476, exon 19: 3477-3693, exon 20: 3694-3791, exon 21: 3792-3909, and exon 22: 3910-3945. The nucleotides not in boldface (nucleotides 3182-3666) correspond to exons 18 and 19 of the predicted transcript. This piece of DNA was amplified from a cDNA library and was used as a template for the production of double stranded RNA (dsRNA) which was used to perform RNA interference (RNAi) against the H06H21.10 gene. RNAi against the dsc-3 gene phenocopied mutant dsc-3.

FIG. 10: The amino acid sequence of DSC-3. The amino acid sequence comprises the predicted amino acid sequence of H06H21.10 from wormbase (www.wormbase.org) and 92 additional amino acids (amino acids 154-245). These 92 amino acids were identified by a tBlastn search of the worm genomic sequence using the sequence of the human ATP8B4 protein as a query.

FIG. 11: Alignment of the amino acid sequences of the gene dsc-3, four homologous Type IV P-Type ATPases from humans, and consensus sequence. FIC1/PFIC1/BRIC corresponds to ATP8B1, which shares highest amino acid identity with ATP8B2 and ATP8B4. The percent identity of dsc-3 (amino acid positions 35-1127) and AT8B1 (amino acid positions 91-1163) is 50%. The percent identity of dsc-3 (amino acid positions 20-1172) and ATP8B2 (amino acid positions 46-1161) is 56%. The percent identity of dsc-3 (amino acid positions 25-812) and ATP8B3 (amino acid positions 194-1034) is 38%. The percent identity of dsc-3 (amino acid positions 137-1115) and ATP8B4 (2 to 946) is 54%.

### 5. DETAILED DESCRIPTION OF THE INVENTION

The invention relates to the use of C. elegans as a model for identifying genes that are involved in lipid metabolism and which can serve as drug targets. The invention is based on the discovery that germline development in clk-1 genetic mutants of C. elegans is uncoupled from somatic development (i.e., heterochronic phenotype), and that reducing the production, or increasing the oxidation of the C. elegans analogue of vertebrate low density lipoprotein (LDL) suppresses this phenotype. These observations, which provide a genetic model system for the study of lipid metabolism, indicate that the oxidation of cellular constituents is reduced in clk-1 mutants and that native lipoproteins inhibit, while oxidized lipoproteins stimulate germline development. Thus, the rate of germline development and other phenotypes of clk-1 mutants can be used to study aspects of lipoprotein metabolism, and lipid metabolism generally. The nematodes and genes also allow the study of the interactions of such genes, environmental factors, such as reactive oxygen species (ROS) and/or drugs in the context of lipoprotein oxidation and its biological effects in a multicellular animal model.

One of the advantages of the drug discovery platform of the invention is that elements of the assays can be combined to produce a variety of assays. Many of the nematodes and their phenotypes used for target identification can also be used to screen for compounds. Based on the genotypes and corresponding phenotypes of the C. elegans mutants of the invention, various assays have been provided to screen for compounds that act on one or more of the drug target genes/gene products to generate a desirable biological outcome which is associated with one or more characteristic phenotypes in the mutant nematodes. Accordingly, the invention is based, in part, on a systematic approach involving in vivo models of a metabolic state or a disorder coupled with sensitive and high throughput gene expression assays. The invention provides a validated platform that permits the discovery of novel genes and gene products (whether novel or known) that are involved in novel pathways that play a role in cardiovascular disease pathology and progression. Thus, the invention allows one to define targets in humans useful for diagnosis, monitoring, drug screening and design, and/or other therapeutic intervention.

In mammals, lipoproteins are essential components in the redistribution of lipids to tissues, either after lipid absorption in the gut, or lipid storage in the liver. In various physiological settings, lipoproteins become oxidized by ROS, which changes their properties. For instance, native LDL is recognized by the LDL receptor, while oxidized LDL (OxLDL) does not bind the LDL receptor, but is recognized by different types of receptors, including scavenger receptors on macrophages. LDL and OxLDL have distinct biological effects on various cell types, including the endothelial cells of the vasculature. In particular, OxLDL has pathobiological significance as it can accumulate in the wall of blood vessels, where it is specifically taken up by macrophages, which thus become foam cells. This process is crucial in the cascade of events that lead to atherosclerosis and is therefore of central significance for human health.

clk-1 encodes a mitochondrial hydroxylase that is required for the biosynthesis of ubiquinone, (UQ, also called coenzyme Q, CoQ), a prenylated benzoquinone lipid that functions as a transporter of electrons in complexes II and III of the respiratory chain. UQ and its reduced form ubiquinol, is a major site of production of reactive oxygen species (ROS). During electron transport, ubisemiquinone species are formed, which are unstable and generate superoxide. Furthermore, ubiquinone/ubiquinol is a redox-active cofactor of other enzyme systems that produce ROS, for example the plasma membrane NAD(P)H oxidoreductases, as well as the lysosomal and peroxisomal electron transport chains. In all these locations-ROS can be produced during redox reactions involving ubiquinone/ubiquinol.

Mutants of the yeast homologue of clk-1, coq-7, do not produce UQ, and therefore cannot grow on non-fermentable carbon sources. Mitochondria isolated from clk-1 mutants also do not contain detectable levels of UQ but instead accumulate the UQ biosynthetic intermediate, demethoxyubiquinone (DMQ) (Miyadera et al., 2001, J Biol Chem 276: 7713-7716). In clk-1 mutants this compound functions as an electron carrier, such that the mitochondria can maintain respiration despite the complete absence of UQ. However, DMQ cannot entirely substitute for UQ as clk-1 mutants cannot complete development when they are fed E. coli strains that do not produce UQ (Jonassen et al., 2001, PNAS 98: 421-426).

In nematodes, the inventors found that the slow germline development of clk-1 mutants, in which UQ is replaced by DMQ, is suppressed by a mutation in dsc-4, the nematode homologue of the large subunit of microsomal triglyceride transfer protein (MTP), which is required for the secretion of apoB-dependent LDL-like lipoproteins. The same effect is obtained by inhibiting the synthesis of the vitellogenins VIT-3, VIT-4 and VIT-5, the worm homologues of apoB, and by disruption of the expression of SOD-1, the cytoplasmic superoxide dismutase, whose detoxifying function is believed to have a major impact on the oxidation of LDL (Guo et al., 2001, Arterioscler Thromb Vasc Biol 21:1131-1138). Based on these findings, a model is provided in which native LDL-like lipoproteins and oxidized LDL-like lipoproteins have opposite effects on the germline, and in which the level of oxidation of these lipoproteins is decreased by the presence of DMQ. UQ functions as a co-factor for a number of systems that produce ROS as well as an antioxidant. It is therefore possible that the effect of DMQ is due to a decrease of ROS production when DMQ is used as a co-factor instead of UQ, or DMQ is a better antioxidant. Without being bound by any particular theory, it has been observed that, like clk-1 mutations, excess vitamin E slows down germline development (Harrington and Harley, 1988), and thus, an increased ability of DMQ over UQ in promoting vitamin E regeneration can explain its effect on germline development.

Although not intending to be bound by any mechanism of action, in essence, some of the phenotypes displayed by clk-1 mutant nematodes (e.g., slow germline development and increased defecation cycle length) are due in part to the accumulation of a pool of native (unoxidized) LDL-like lipoprotein. Therefore, such clk-1 mutant phenotypes can be rescued in nematodes by certain processes that reduce native LDL-like lipoprotein levels. This can be accomplished in a variety of processes including decreasing native LDL-like lipoprotein synthesis/secretion or increasing the conversion of native to oxidized LDL-like lipoprotein. Examples provided herein below have provided a proof of principle by restoring wild type phenotypes in clk-1 mutant nematodes through manipulation of native LDL-like lipoprotein levels. In one approach, LDL-like lipoprotein synthesis/secretion was decreased in nematodes by lowering the availability of either the major dietary lipid (i.e., cholesterol) or protein component (i.e., apoB homologs vit-3, vit-4 and vit-5) of LDL. Both cholesterol depletion (see Section 8.3) and vit-3, vit-4 and vit-5 RNAi administrations (see Section 8.2) caused a change in the rate of germline development that was essentially wild type. Alternatively, LDL levels were decreased by increasing conversion of native LDL to oxidized LDL. Production of oxidized LDL was increased by increasing reactive oxygen species (ROS) available for interaction with native LDL. ROS levels were increased by decreasing the function of an antioxidant enzyme superoxide dismutase (SOD) by the administration of SOD RNAi (Section 8.4). Decreased native LDL levels due to increased oxidized LDL also caused a change in the rate of germline development that was essentially wild type.

The identification of dsc-3 mutants as suppressor mutants of clk-1 and the effect of cholesterol depletion on clk-1 mutants further indicate that the clk-1 phenotype is sensitive to changes in the levels of certain lipids, such as cholesterol and related metabolites. A change in the level of such lipids can contribute to a change in native LDL-like lipoprotein level thereby affecting the phenotype of the nematode.

In one embodiment, the invention provides the use of clk-1 mutants of C. elegans to identify novel genes that are involved in lipid metabolism, particularly changes in cholesterol levels, LDL secretion and LDL oxidation. The phenotype of clk-1 mutants is pleiotropic, with most aspects of development, behavior and reproduction being slowed on average, including the embryonic cell cycles, overall embryonic development, post-embryonic development, various cyclic behaviors, such as defecation, pharyngeal pumping, and swimming, as well as the egg-laying rate and aging (Wong et al., 1995, Genetics, 139:1247-1259). According to the method of the invention, by generating and isolating mutants that suppress the various phenotypes of clk-1 mutants under different environmental conditions, novel classes of genes can be identified that affect particular aspects of clk-1 functions. Phenotypes that can be used in identifying and characterizing the suppressor mutants include, e.g., length of defecation cycle, rate of germline development, and embryonic and post-embryonic development. The methods of the invention rely on the correlation of such phenotypes with changes in the levels of lipids and/or lipoproteins in test nematodes. Detailed description of these assays based on functional analysis are provided in Section 5.4.

In a related aspect, the methods can also be used to investigate and validate the functions of C. elegans genes that share structural elements and/or sequence homology with vertebrate genes that are involved in lipid metabolism. The methods of the invention for identifying and characterizing novel gene targets are described in details in Sections 5.3.1 and 6.

In another related aspect, the methods can also be used to identify proteins in C. elegans which can be oxidized by ROS, particularly those molecules of which the oxidized forms possess different biological properties and as a result lead to changes in the phenotype of the nematode.

In yet another embodiment, the invention provides novel genes and gene products that suppress the phenotypes of clk-1 mutants. These genes are identified by the methods of the invention and can fall into several classes depending on the aspects of the phenotype of clk-1 mutants that were modified. Also provided are nematodes containing a mutation in any one of these genes, and their uses in biological assays and drug screening assays. In a related embodiment, the invention also provides “humanized” nematode in which the human homolog of the nematode genes identified by the methods of the invention are cloned into and expressed in nematodes. The use of such nematodes in biological assays and drug screening assays are contemplated.

In a specific embodiment, the invention provides the dsc-4 gene of C. elegans and the dsc-4 gene product, which was found to be the nematode homologue of vertebrate MTP. The experimental results described in Section 7 indicate that structural elements involved in the production and oxidation of LDL-like lipoproteins exist in nematodes and are functionally related in a way similar to that observed in vertebrates. It was observed that, as in vertebrates, reduction of cholesterol intake reduces LDL-like lipoprotein secretion and that ROS lead to their oxidation. The findings indicate that the effect of oxidized LDL-like lipoprotein in nematode germline development is analogous to the biological effects of oxidized LDL on vertebrates that lead to atherosclerosis.

In another specific embodiment, using the gene identification methods described herein, the inventors identified a C. elegans gene, designated herein as dsc-3. A mutation in dsc-3 suppresses the slow defecation phenotype of clk-1 mutants. The effect of a dsc-3 mutation is not additive to that of a dsc-4 mutation suggesting that the two mutations act in the same pathway or affect the same process. Based on sequence homology, the gene product of dsc-3, DSC-3, is a member of a family of type IV P-type ATPase, and in particular, ATP-dependent amino-phospholipid transporters. One of the dsc-3 homologs in human is the human gene ATP8B 1 which is deficient in patients suffering an autosomal-recessive familial liver disorder characterized by intrahepatic cholestasis, i.e., the impairment of normal bile flow without anatomical obstruction. The mechanism by which the absence or dysfunction of ATP8B1 in humans leads to cholestasis is currently undefined. Since the cholesterol level in mammals is regulated by the coordinate regulation of intestinal absorption, endogenous synthesis, and biliary secretion, the identification of dsc-3 by the method of the invention indicates, through its action on bile acid regulation, that it plays a role in cholesterol homeostasis in nematodes as well as in mammals.

In various embodiments, the invention provides correlations of phenotypes in nematodes with levels of lipids and/or liporproteins and corresponding gene activities. And because the functions of the homologs of such genes are known in mammals, the invention further provides the recognition of nematode genes (such as dsc-3, dsc-4 and others) and their homologs identified by the methods of the invention, as excellent drug screening targets as well as candidates for genes that are mutated or become deregulated in human disorders related to lipid metabolism. Other members of this class of genes, dsc-1, dsc-2, dsc-5, dsc-7, and dec-7 and their uses in various methods described herein are also encompassed by the invention.

Accordingly, the invention encompasses the use of nucleic acids, such as the dsc class of genes including dsc-3 and dsc-4 which are exemplary members, in genetic analysis, mutagenesis, recombinant expression, assays such as diagnostic assays, gene therapy, and transgenic experiments. Details of the nucleotide sequences of the nucleic acids of the invention and their uses are described in Section 5.1.1.

The invention also encompasses the use of dsc gene products, such as DSC-3 and DSC-4 polypeptides, in antibody generation, protein engineering (including fusion with other proteins), therapy, and various assays, including biological assays and drug screening assays. Details of the amino acid sequences of the polypeptides of the invention and their uses are described in Section 5.1.2.

Also encompassed are mutant nematodes containing mutations in one or more genes of the invention, and mutations of known C. elegans genes including clk-1. In specific embodiments, the invention provides nematode mutants comprising at least one mutation in dsc-4 and/or dsc-3, or nematodes in which the normal level of dsc-4 and/or dsc-3 expression is reduced, or mutants comprising mutations in at least two genes, such as clk-1/dsc-3 and clk-1/dsc-4 double mutants. Details on the compositions and methods of making and using mutant nematodes are provided in Section 5.3.1.

The invention also encompasses the use of dsc-3 nucleic acids, DSC-3 polypeptides, dsc-4 nucleic acids, DSC-4 polypeptides, and/or the aforementioned mutant nematodes in various methods of genetic and biological analysis, and screening. The uses of dsc-3 genes, DSC-3 polypeptides, dsc-4 genes and DSC-4 polypeptides as a drug targets are specifically provided. In a related aspect, the use of dsc-3 mutants and/or dsc-4 mutants in the assays of the invention to identify additional genes involved in lipoprotein metabolism are also contemplated.

One of the main objectives of the present invention is to provide methods for the selection of compounds for use in the field of metabolism disorders including but not limited to cardiovascular diseases and dyslipidemia disorders. The invention features a platform for screening drugs useful in the treatment and prevention of such metabolism disorders in humans. Various assays are provided to screen for compounds that generate a desirable biological outcome which is associated with one or more characteristic phenotypes in the test nematodes. Phenotypes such as embryonic cell cycles, overall embryonic development, post-embryonic development, and various cyclic behaviors, e.g., defecation, pharyngeal pumping, and swimming, egg-laying rate and aging can be used. Essentially, the phenotypes of the test nematodes are used as biological read-out in the assays of the invention for the activity of one or more target genes/gene products or the level of certain lipids and/or lipoproteins. The test compounds may act on initially unknown drug target genes/gene products in the test nematodes. For example, the invention provides assays based on germline development of test nematodes for screening compounds that reduce the level of LDL secretion and/or LDL oxidation in vertebrates, preferably humans. The phenotypes of the nematode can also indicate the level of certain lipid metabolites in the nematodes which reflect the activities of the target gene products. The methods of the invention may also be used for other drug development needs, such as but not limited to dereplication, and pharmacological and toxicity studies. Details of the compound screening assays of the invention are provided in Section 5.3.2.

In a related aspect, the invention provides assays for screening compounds that can modulate lipid transport and/or lipoprotein transport, including the transport of fatty acids, sterols (e.g., cholesterol), and primary and secondary bile salts and acids. The C. elegans genome comprises several vit genes that resemble the apoB protein found in VLDL and LDL lipoproteins. The vitellogenins are major constituents of egg yolk in a variety of organisms including nematode worms. In mammals, the formation and lipidation of LDL-like lipoproteins requires the activity of the microsomal triglyceride transfer protein (MTP), which is homolgous to DSC-4. A decrease of the synthesis and activity of a C. elegans DSC-4 produces specific biological effects but does not or only marginally, affect the synthesis of egg yolk. These results indicate that LDL-like lipoproteins assembled in the endoplasmic reticulum (ER) represent a small subset of apoB-containing lipoproteins in C. elegans. Without being bound by any theory, it follows that apoB-containing yolk particles can be assembled extracellularly as are a majority of lipoproteins in some others organisms. The MTP-dependent, ER-assembled lipoproteins would likely have a different structure from other apoB-containing lipoproteins. It is contemplated that in C. elegans, even in view of the dominant presence of yolk-like lipoproteins for oocyte production, there exists a variety of other particles, as in mammals, comprising one or more lipoproteins that are involved in lipid transport, such as but not limited to high density lipoprotein (HDL)-like particles, very low density lipoprotein (VLDL)-like particles. The use of these complex nematode lipoprotein particles as targets in the assays of the invention are encompassed. It is also contemplated that the dsc genes of the invention can encode components of such particles or enzymes involved in the synthesis, assembly and transport of such lipoprotein particles in the nematode.

In another specific embodiment, the invention provides assays for screening compounds that can modulate the state of oxidation of biological entities in vivo, including but not limited to cells, organelles, cellular constituents, cell surface components, extracellular materials, lipids, proteins, carbohydrates, and nucleic acids. An example of such an entity is LDL.

In a related aspect, the invention provides assays in which the morphologic, behavioral and developmental phenotypes of the nematodes are not assessed by visual observation. These assays are based on gene expression profiles associated with the phenotypes and/or the use of reporter gene constructs comprising C. elegans promoters of which the activities are associated with the phenotypes. In another related aspect, the invention also provides assays in which the levels of lipids and/or lipoproteins in nematodes are directed observed and/or measured. The above-described visual and non-visual assays form an integral part of the platform for screening and analyzing the nematodes of the invention. Some of the assays of the invention, especially the non-visual assays have been developed such that many or all of the steps can be automated.

Active compounds identified by the assays of the invention are for pharmaceutical, veterinary or agrochemical/pesticidal (e.g. insecticidal and/or nematocidal) use. The active compounds can be used in vertebrates, preferably mammals such as companion animals, farm animals, and protected wild animals, and most preferably humans. Other embodiments, uses, benefits of the system will become apparent from the further descriptions provided herein below.

### 5.1 Modulators of Lipid and Lipoprotein Levels

The present invention relates to the identification of C. elegans genes that are involved in lipid metabolism and which can serve as drug targets. Such genes are generically termed Modulators Of Lipids and Lipoproteins (or MOLL), and include genes that can modulate the levels of lipids and lipoprotein in C. elegans. MOLL nucleic acids and/or polypeptides have a role in lipid metabolism including, but not limited to, i) lipoprotein synthesis/secretion, ii) bile acid/salt synthesis, absorption and excretion; (iii) modulation of lipid levels generally such that more or less lipids are available for incorporation into lipoprotein or conversion to bile acid/salts; (iv) sterol synthesis, transport and utilization, v) LDL oxidation, vi) ROS production, and/or vii) ROS clearance. The involvement of MOLLs in lipid metabolism can be direct (e.g., a polypeptide that is a lipoprotein or a component of a lipoprotein complex, an enzyme that oxidizes LDL, a polypeptide that transport a lipid from one location to another within the body or within a cell, etc.) or indirect (e.g., a polypeptide that causes a change in activity level of a polypeptide that is directly involved in lipid metabolism, etc.). Accordingly, the levels of many different lipids, lipoproteins and their metabolites are expected to be modulated in the test nematodes and genetic models of the invention. See Watts and Browse, 2002, PNAS 99:5854-5859, Watts et al., 2003, Genetics 163:581-589, Lesa et al., 2003, J. Cell Sci. 116:4965-4975, Kniazeva et al., 2003, Genetics 163:159-169. Examples of such lipids include but are not limited to C-12 fatty acids (e.g., lauric acid), C-14 fatty acids (e.g., myristic acid), C-16 fatty acids (e.g., palmitic acid), C-18 fatty acids(e.g., stearic acid), C-20 fatty acids (e.g., arachidonic acid) and C-22 fatty acids (e.g., cervonic acid); ubiquinone and related lipids involved in electron transport; sterols (e.g., cholesterol), oxysterols (e.g., 22(R)-hydroxyl cholesterol), phytosterols (e.g., campesterol, sitosterol, stigasterol); as well as intermediates of cholesterol biosynthesis starting from lanosterol and intermediates of cholesterol utilization from cholesterol, 7-hydroxycholesterol through to primary and secondary bile acids/salts.

Examples of MOLLs include dsc-1, dsc-2, dsc-3, dsc-4, dsc-5, dsc-7, or dec-7. In a preferred embodiment, the invention provides dsc-3 and dsc-4 as exemplary MOLL nucleic acids.

Mutant MOLLs can be identified in the target screens of the invention as suppressors of a clk-1 mutant phenotype in C. elegans resulting from undesirable levels of native LDL or ROS (e.g., defecation cycle length, heterochronic germline development, rate of embryonic or post-embryonic development). In one embodiment, mutant MOLLs can be used to isolate wild type MOLL homologues in C. elegans as well as in other species (e.g., humans). In another embodiment, mutant MOLLs can be introduced into C. elegans and be used in further target screens (in place of mutant clk-1) to identify additional MOLLs or in drug screening assays to identify agents of the invention. In some embodiments, a C. elegans that has one mutant MOLL is used to screen for additional targets or agents of the invention. In other embodiments, a C. elegans that has more than one mutant MOLL is used to screen for additional targets or agents of the invention.

5.1.1 MOLL Nucleic Acids

The present invention encompasses MOLL nucleic acids (such as the dsc-4 and dsc-3 nucleic acids set forth in SEQ ID NO:1 and 7 respectively). The inventors, as a proof of principle, have conducted a screen of the invention to identify MOLLs and have identified dsc-4 as a first example of a MOLL. All mutant MOLL nucleic acids identified in the screening methods of the invention as well as their wild type counterparts are nucleic acids of the invention. In addition, it will be appreciated that nucleic acids of the invention also encompass variants of MOLL nucleotide sequences of the invention, including, but not limited to, any fragment, homologue, naturally occurring allele, or mutant thereof. Nucleic acids of the invention also encompass those nucleic acids capable of hybridization to the MOLL nucleic acids under stringent conditions. Nucleic acids of the invention also encompass those nucleic acids capable of encoding the same polypeptide as the MOLL nucleic acid as well as those nucleic acids that can hybridize under stringent conditions to those nucleic acids capable of encoding the same polypeptides as the MOLL nucleic acids. One or more activities of polypeptides encoded by nucleic acids of the invention can vary relative to the activities of the polypeptides encoded by MOLL nucleic acids identified by the methods of the invention.

In one embodiment, the invention provides nucleic acids that encode the amino acid sequence of a MOLL polypeptide. The invention also provides nucleic acids comprising a nucleotide sequence that encode the amino acid sequence of a MOLL polypeptide, such as the amino acid sequences of SEQ ID NO:2 or 8 or a fragment thereof. In a specific embodiment, the nucleic acids do not comprise intron sequences, or genomic sequences that are contiguous to the nucleotide sequence set forth in SEQ ID NO:1 or 7 in the C. elegans genome, or genomic sequences that are contiguous to subsequences of SEQ ID NO:1 or 7 which correspond to individual exons in the C. elegans genome. In another specific embodiment, the nucleic acid in the nucleic acid clones designated yk357a6, K02D7, H06H21, or Y17G9 are not encompassed by the invention.

In another embodiment, nucleic acids that are at least 75%, 80%, 85%, 90%, 95%, 98%, or 99% identical to the MOLL nucleotide sequence (e.g., SEQ ID NO:1 or 7) or variants thereof are encompassed by the invention. To determine the percent identity of two nucleic acid sequences, the sequences are aligned for optimal comparison purposes (e.g., gaps can be introduced in the sequence of a first nucleic acid sequence for optimal alignment with a second or nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, PNAS 87:2264-2268, modified as in Karlin and Altschul, 1993, PNAS 90:5873-5877. Such an algorithm is incorporated into the NBLAST and XBLAST programs of Altschul et al., 1990, J Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of NBLAST) can be used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted. Preferably, the sequence comparison is performed along the entire length of at least one of the nucleic acid sequences, usually the sequence of a nucleic acid probe.

In another embodiment, fragments of MOLL nucleic acids or variants thereof are encompassed by the invention. The invention features nucleic acid molecules which comprise a fragment of at least 100, 200, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, or 4100, contiguous nucleotides of the MOLL nucleotide sequence identified by the methods of the invention (e.g., SEQ ID NO:1 or 7) or variants or complement thereof. In a preferred embodiment, the fragment comprises at least a portion of the open reading frame or coding sequence. In another embodiment, the fragment encodes one or more exons and/or domains (e.g., functional domains) of the MOLL polypeptide of the invention.

In a less preferred embodiment, the MOLL nucleic acids of the invention do not encompasses isolated clones or fragments of genomic DNA that comprises a MOLL nucleic acid or a fragment thereof, and that are from a genomic DNA library generated by cloning of total or chromosome-specific genomic DNA.

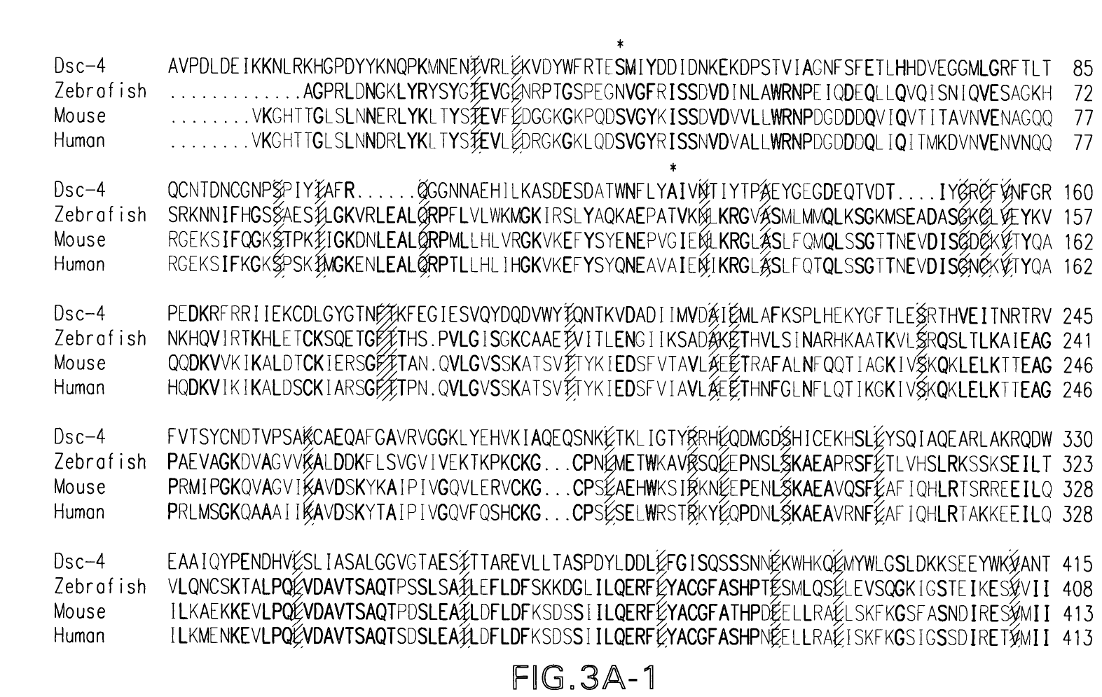

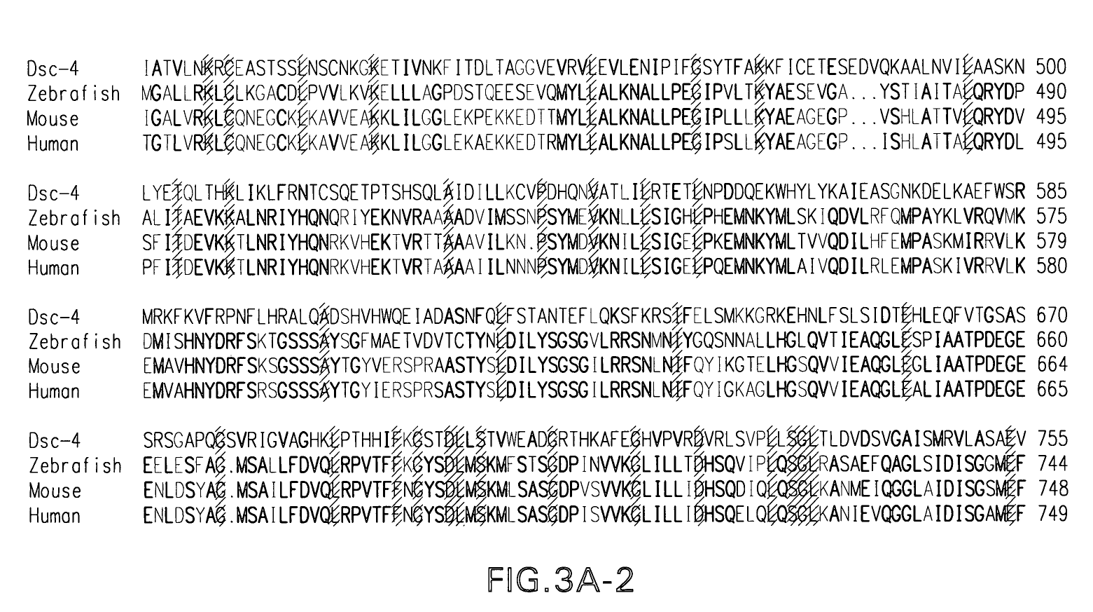

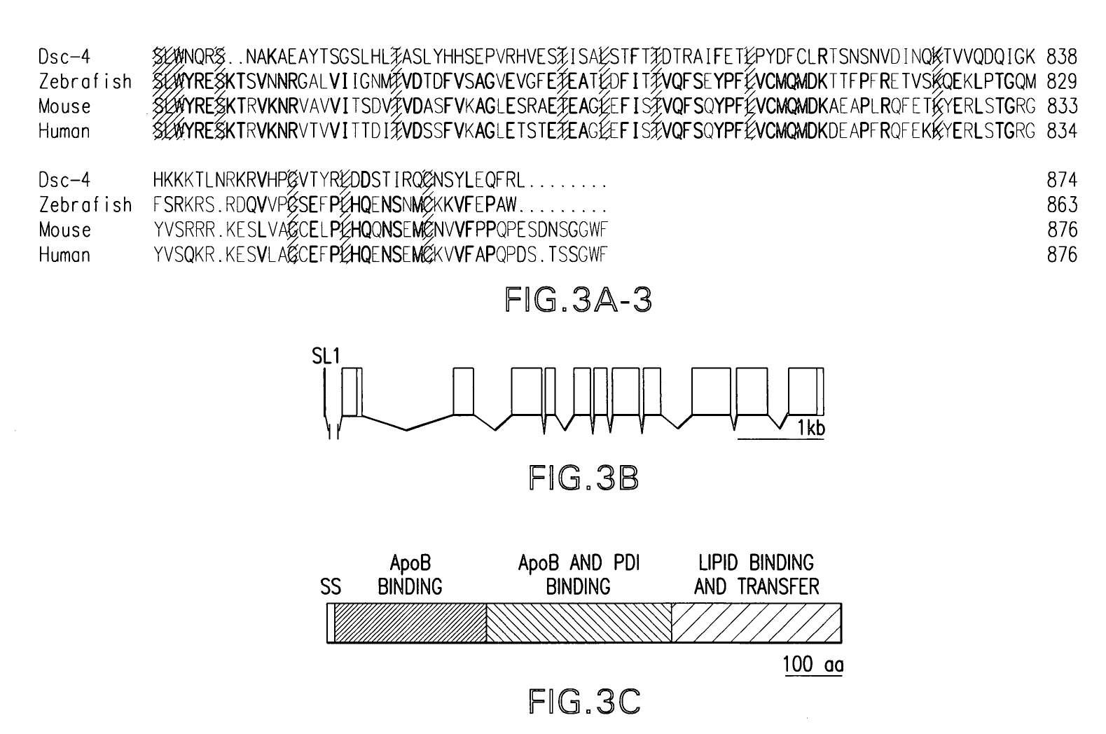

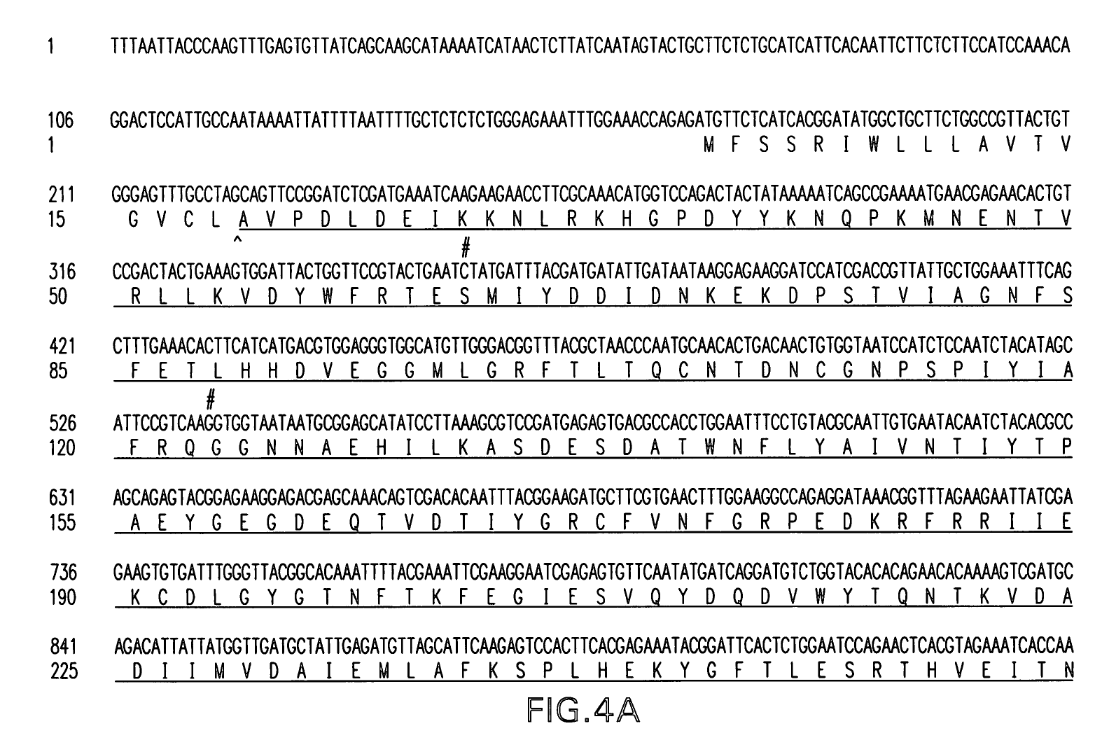

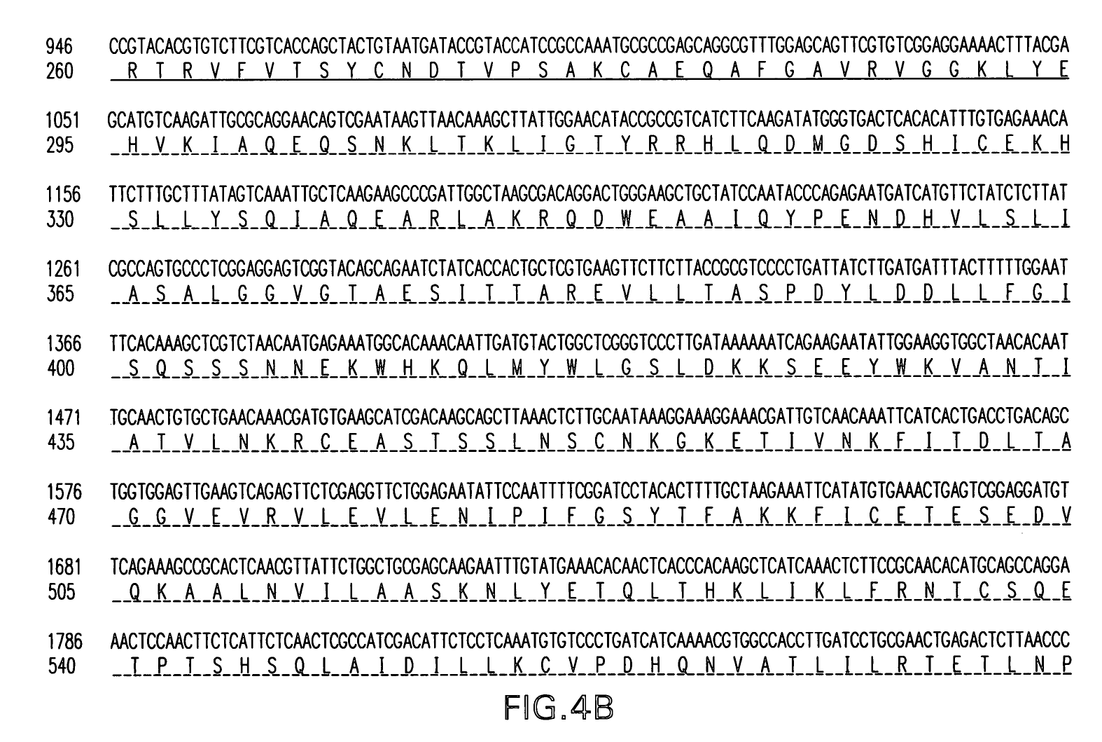

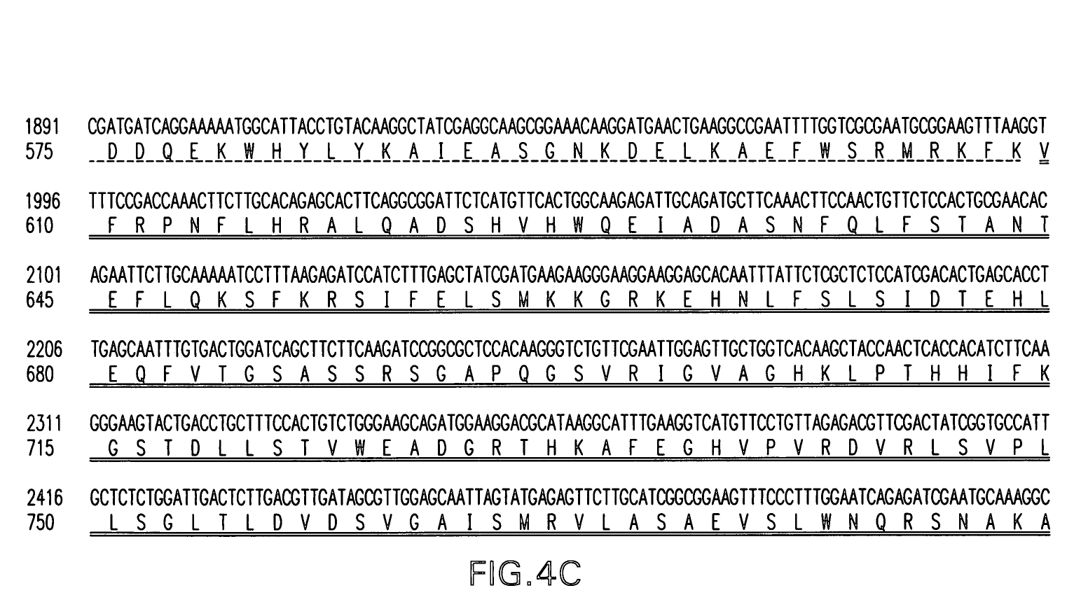

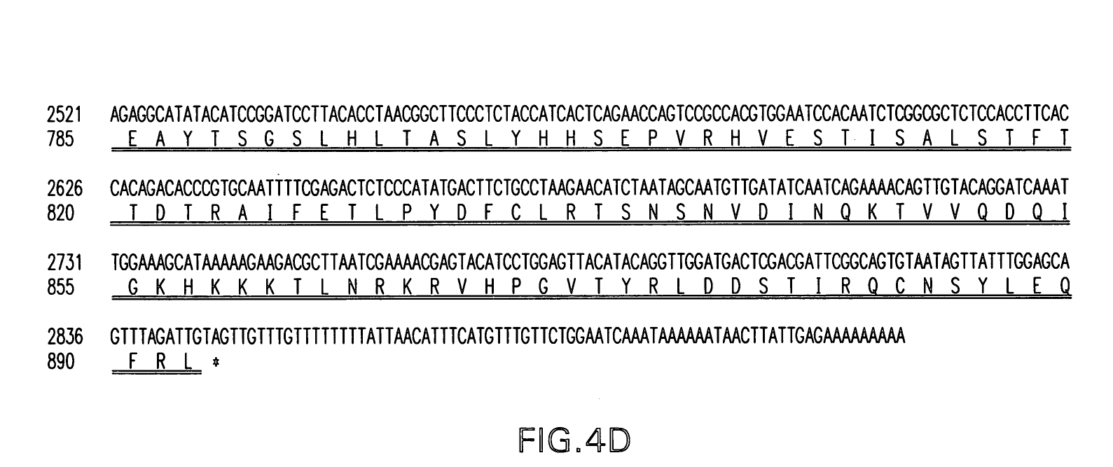

In a specific embodiment, where the MOLL is dsc-4, the nucleic acid fragment encodes one or more of the apoB binding domain, the PDI binding domain, or the lipid binding domain of dsc-4 (see e.g., FIGS. 3C and 4).

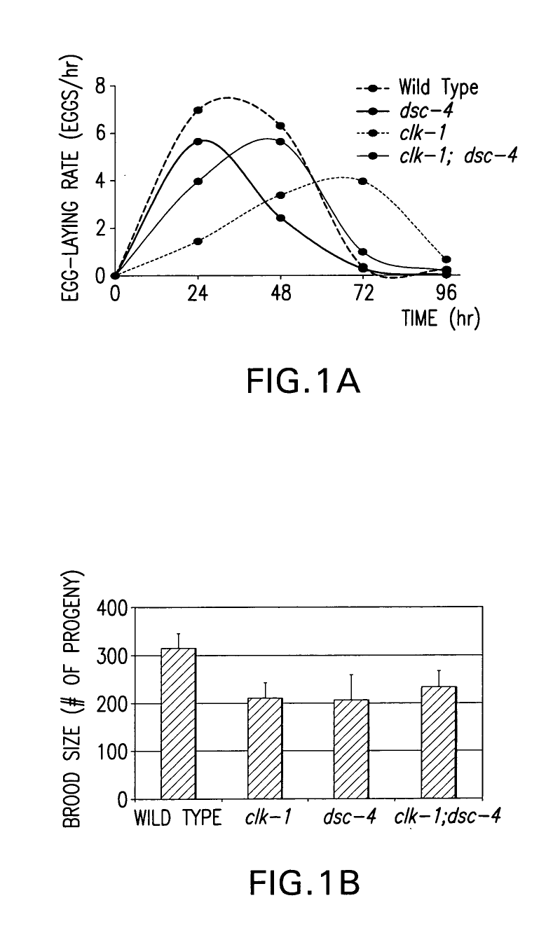

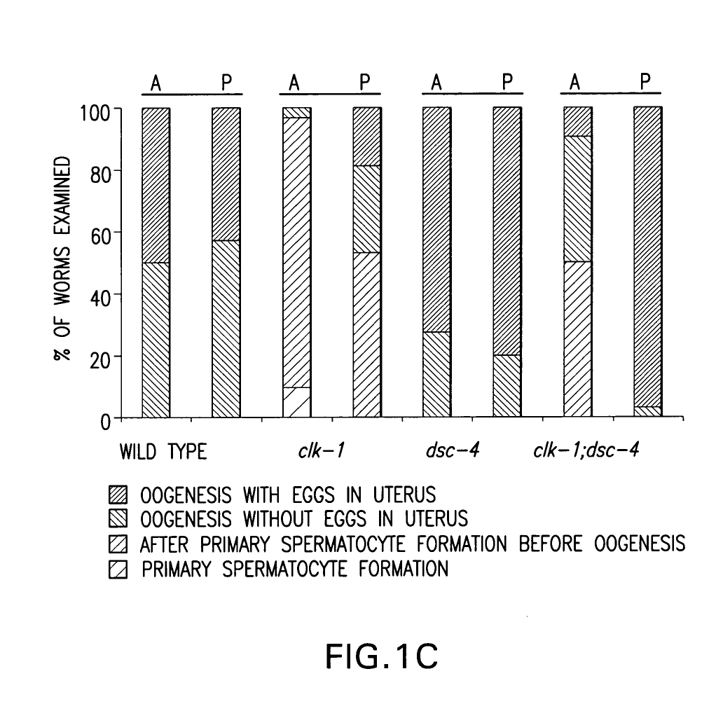

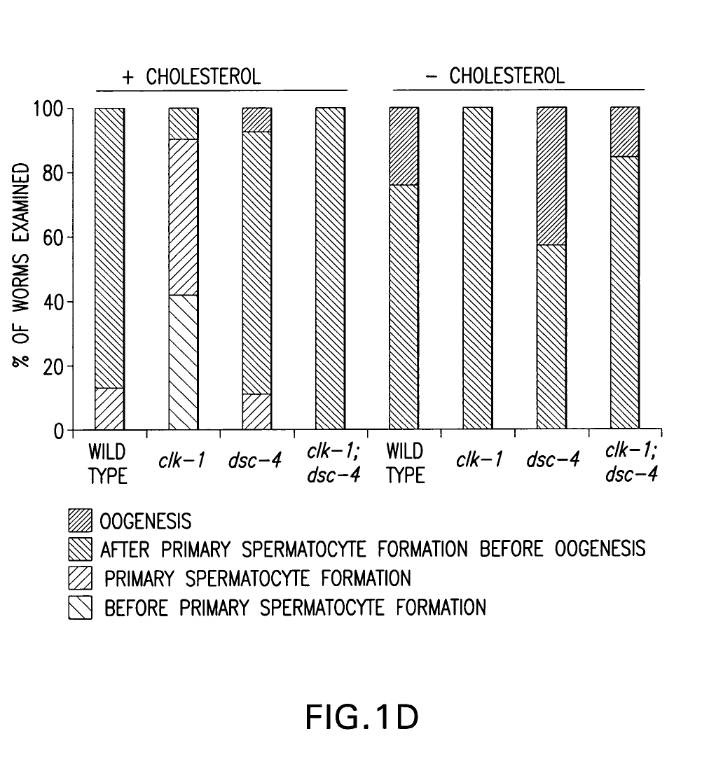

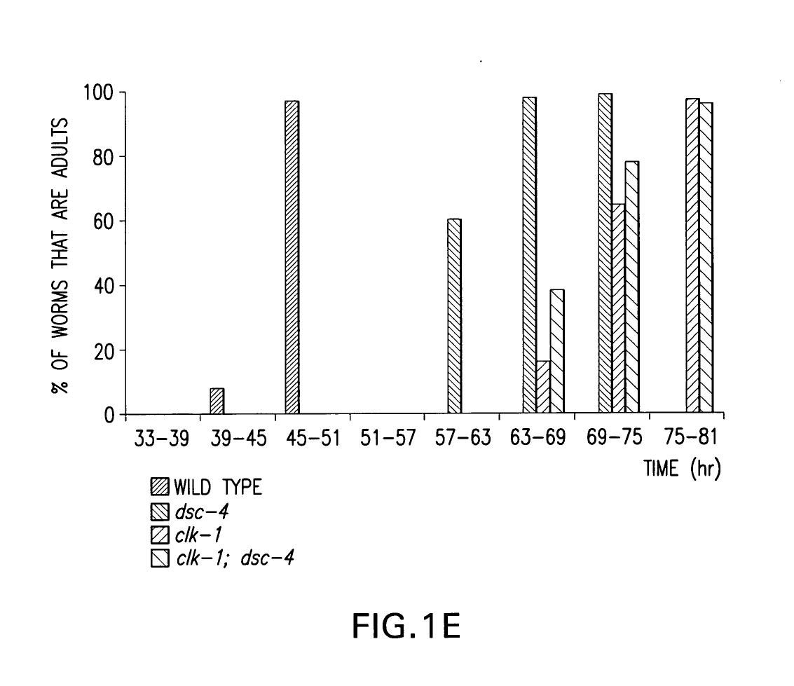

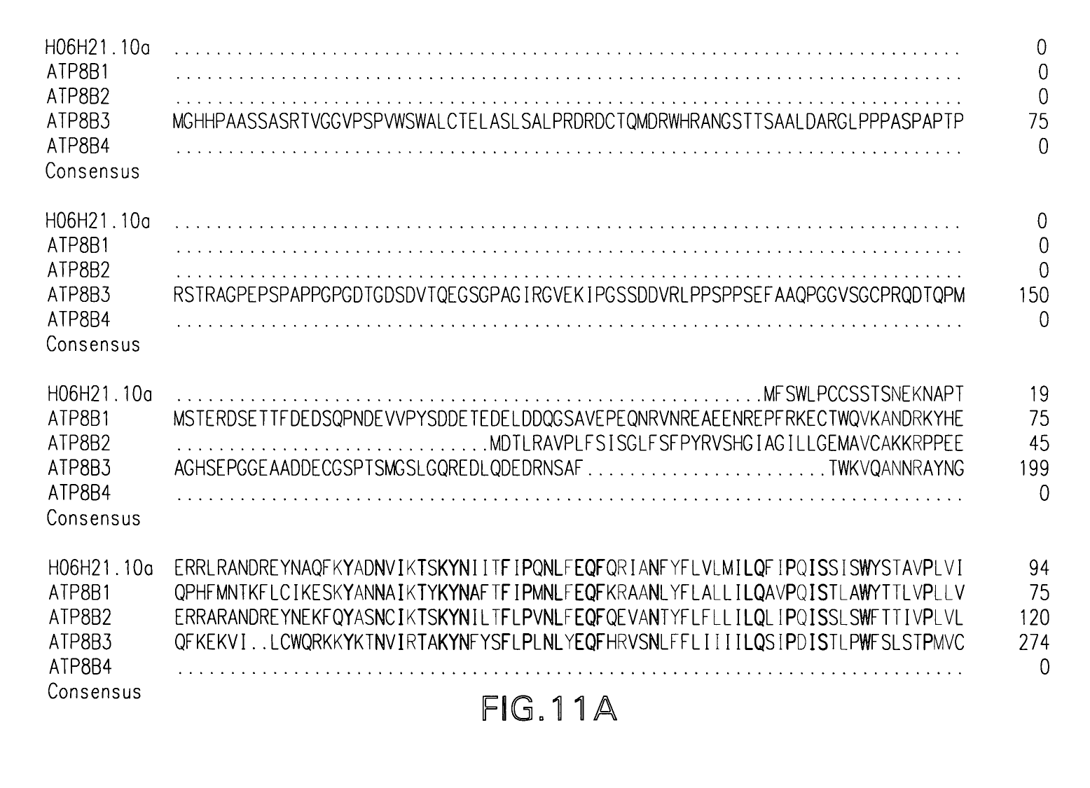

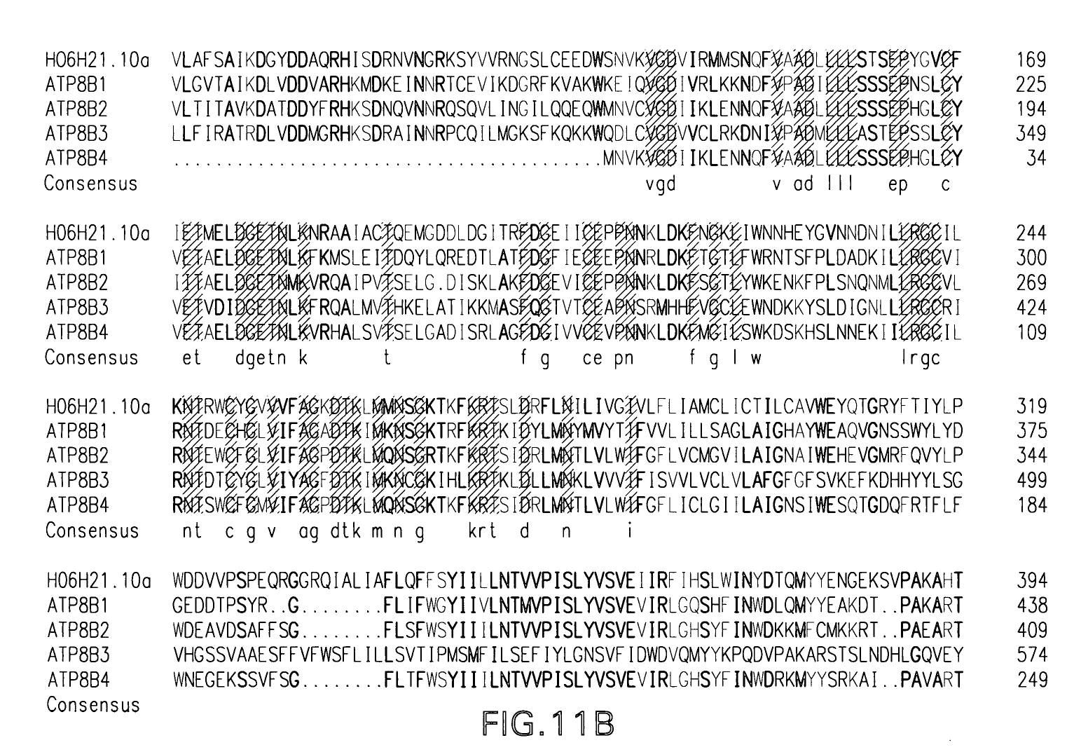

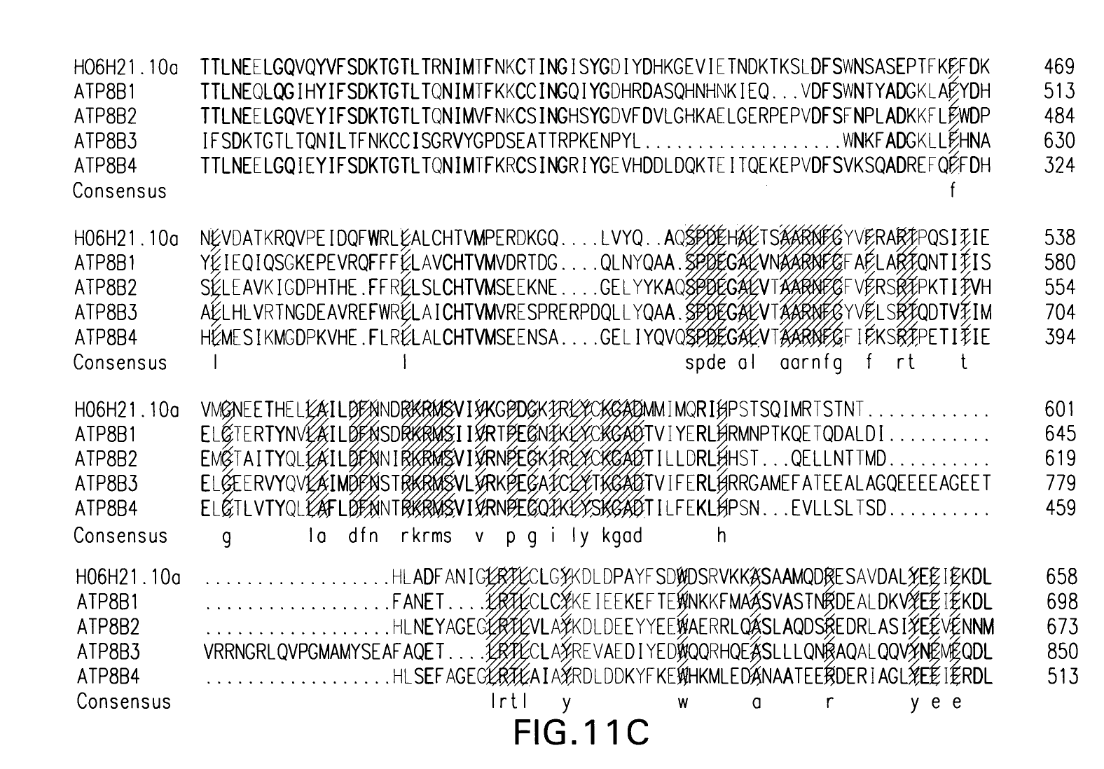

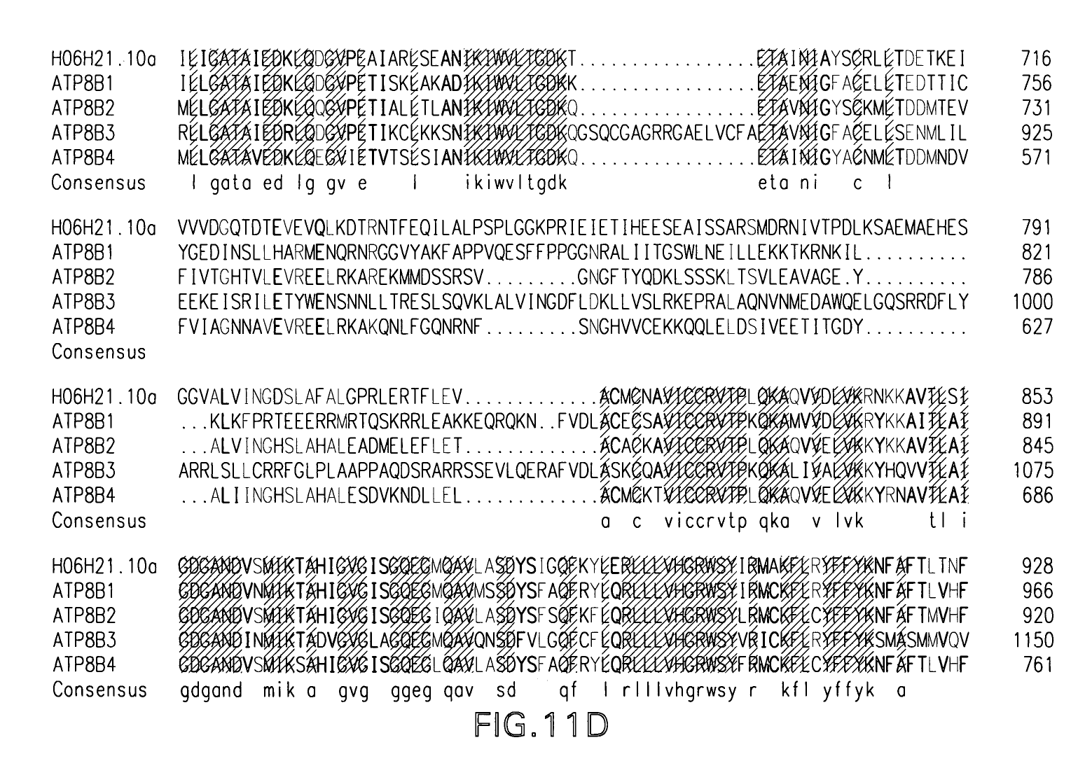

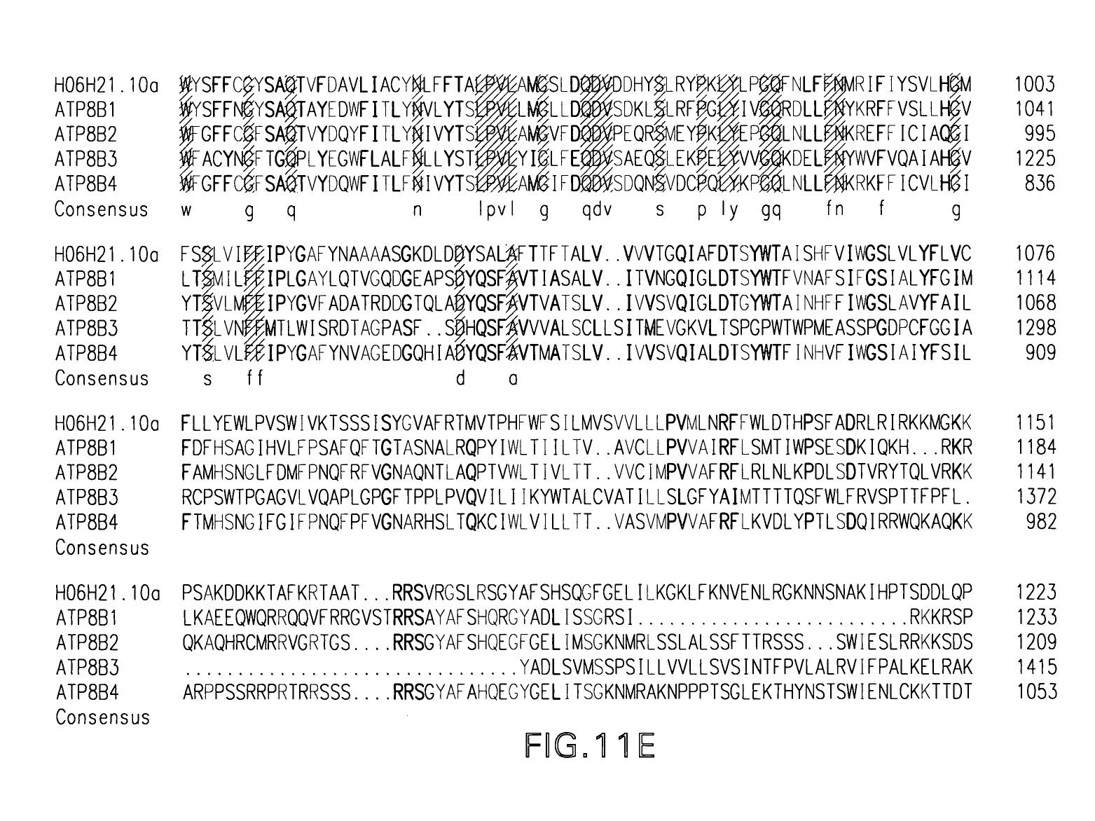

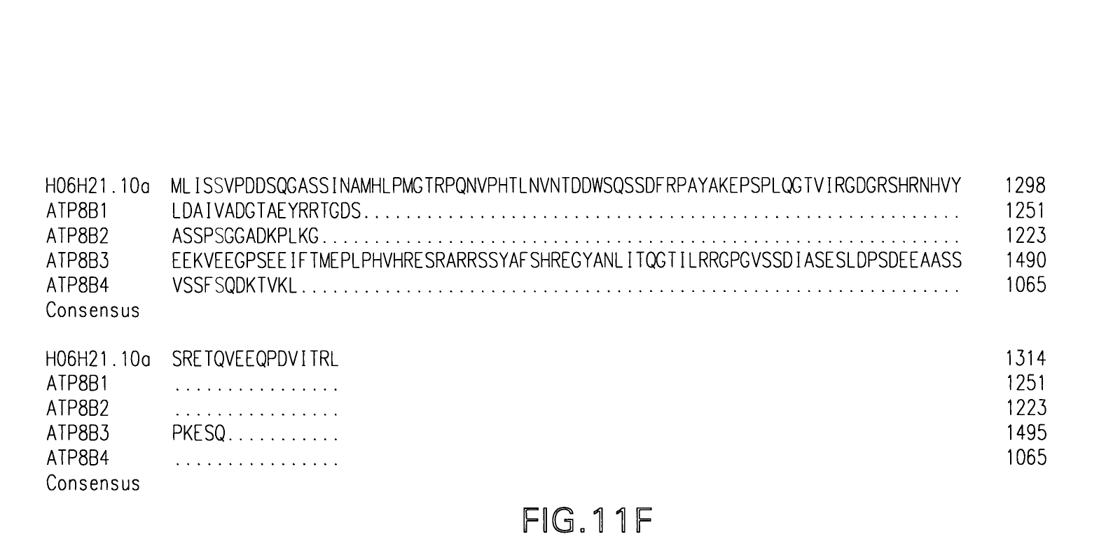

In another specific embodiment, where the MOLL is dsc-3, the nucleic acid fragment encodes one or more of the domains that align with the eight conserved domains as defined in FIG. 1 of Harris et al. (2003, Biochem Biophys Acta 1633:127-131, which is incorporated herein by reference in its entirety), or one of the transmembrane domains. (FIG. 11). The nucleic acid fragments can also encode peptides comprising one or more of the functional domains of DSC-3, such as the phosphorylation domain in the large cytoplasmic loop, the ATP binding domains, the ATP hydrolysis domain, and/or the hinge domain that connects the ATP binding sites to conserved residues within the transmembrane domains for substrate translocation. Examples of nucleic acid fragments of the invention are further described in Sections 7 and 9.

Those skilled in the art will recognize that nucleic acid sequence polymorphisms that may or may not lead to changes in the encoded amino acid sequence may exist within a population (e.g., the human population). Such genetic polymorphisms may exist among individuals within a population due to natural allelic variation. An allele is one of a group of genes which occur alternatively at a given genetic locus. As used herein, the phrase “allelic variant” refers to a nucleotide sequence which occurs at a given locus or to a polypeptide encoded by the nucleotide sequence. As used herein, a “naturally-occurring” nucleic acid molecule refers to an RNA or DNA molecule having a nucleotide sequence that occurs in nature (e.g., encodes a natural protein). Naturally-occurring allelic variations can typically result in 1-5% variance in the nucleotide sequence of a given gene. Usually naturally occurring variations do not alter or do not substantially alter the functional activity of the encoded polypeptide. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation are intended to be within the scope of the invention. In one embodiment, polymorphisms that are associated with a particular disorder are used as markers to diagnose said disorder.

Moreover, nucleic acid molecules encoding proteins of the invention from C. elegans and other species (homologs) which have a nucleotide sequence which differs from that of the C. elegans protein (e.g., human) are intended to be within the scope of the invention. Nucleic acid molecules corresponding to natural allelic variants and homologs of a nucleic acid of the invention can be isolated based on their identity to the C. elegans or human nucleic acid molecule using the C. elegans or human nucleic acid, or a portion thereof, as a hybridization probe according to standard hybridization techniques under stringent hybridization conditions.

Accordingly, in another embodiment, an isolated nucleic acid molecule of the invention is at least 25, 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000 or 4100 contiguous nucleotides in length and hybridizes under stringent conditions to the nucleic acid molecule comprising the nucleotide sequence, preferably the coding sequence, of a MOLL nucleic acid or a complement thereof.

In addition to naturally-occurring allelic variants of a nucleic acid molecule of the invention, the skilled artisan will further appreciate that changes can be introduced by mutation into the nucleotide sequence of a nucleic acid of the invention that may or may not result in changes in the amino acid sequence of the encoded protein, either with or without altering the biological activity of the protein. Such mutant nucleic acids are also encompassed in the invention.

Accordingly, in another embodiment, the invention pertains to nucleic acid molecules encoding a polypeptide of the invention that contain changes in amino acid residues that may or may not be essential for at least one activity. Such polypeptides differ in amino acid sequence from polypeptides encoded by MOLL nucleic acids yet retain at least one biological activity.

Another aspect of the invention pertains to nucleic acid molecules that encode polypeptides that include an amino acid sequence that is at least about 30%, 35%, 40%, 45%, 50%, 55%, 60%, 65%, 75%, 85%, 95%, or 98% identical to the amino acid sequence of a MOLL polypeptide. As used herein, the term “hybridizes under stringent conditions” is intended to describe conditions for hybridization and washing under which nucleotide sequences at least 60%, 65%, 70%, 75%, 80%, 85%, 90% identical to each other typically remain hybridized to each other. Such stringent conditions are known to those skilled in the art and can be found in, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3. A preferred, non-limiting example of stringent hybridization conditions are hybridization in 6× sodium chloride/sodium citrate (SSC) at about 45° C. followed by one or more washes in 0.2×SSC, 0.1% SDS at 50-65° C. Highly stringent conditions such as hybridization to filter-bound DNA in 6X SSC at about 45° C. followed by one or more washes in 0.1×SSC/0.2% SDS at about 60° C. can also be used in the invention.

In one embodiment, MOLL nucleic acids can be used as probes to monitor expression levels of MOLL genes. In a specific embodiment, MOLL nucleic acid expression is used to diagnose disorders involving undesirable levels of lipids, bile salts/acids, lipoproteins (including but not limited to LDL) and/or ROS. In another specific embodiment, MOLL nucleic acid expression is used to monitor effectiveness of treatment (either MOLL based or non-MOLL based treatment) of disorders involving undesirable levels of lipids, bile salts/acids, lipoproteins (including but not limited to LDL) and/or ROS. In another specific embodiment, MOLL nucleic acid expression is used to predict those individuals predisposed or likely to suffer from a disorder involving undesirable levels of lipids, bile salts/acids, lipoproteins (including but not limited to LDL) and/or ROS.

In another embodiment, MOLL nucleic acids can be used to recombinantly express MOLL polypeptides. The full length MOLL or any portion or domain thereof can be expressed and if desired, purified by conventional techniques. Additionally, fusion proteins can be created by expressing a fusion construct wherein a MOLL nucleic acid is joined to a nucleic acid encoding a heterologous polypeptide or portion thereof.

In another embodiment, MOLL nucleic acids are agents of the invention to be used in methods of treatment of disorders involving undesirable levels of lipids, bile salts/acids, lipoproteins (including but not limited to LDL) and/or ROS. For example, MOLL nucleic acids can be used to increase MOLL expression (i.e., gene therapy) or decrease MOLL expression (i.e., MOLL antisense) in vivo.

5.1.2 MOLL Polypeptides

The present invention encompasses MOLL polypeptides, such as DSC-4 and DSC-3, the amino acid sequences of which are set forth in SEQ ID NO:2 and SEQ ID NO:8 respectively. The mutant MOLL polypeptides identified in the screening methods of the invention as well as their wild type counterparts are polypeptides of the invention. In addition, it will be appreciated that polypeptides of the invention also encompass variants of MOLL polypeptides of the invention, including, but not limited to, any fragment, derivative, homolog, naturally-occurring allele, or mutant thereof. Polypeptides of the invention also encompass those polypeptides that are encoded by any of the nucleic acids described in Section 5.1.1.

As used herein, the term “derivative” refers to a polypeptide that comprises an amino acid sequence of a MOLL polypeptide of the invention (e.g., dsc-4) which has been altered by the introduction of amino acid residue substitutions, deletions or additions. Derivative polypeptides may or may not possess residues that have been modified, i.e., by the covalent attachment of any type of molecule to the polypeptide. For example, but not by way of limitation, a derivative polypeptide of the invention may be modified, e.g., by glycosylation, acetylation, pegylation, phosphorylation, amidation, derivatization by known protecting/blocking groups, proteolytic cleavage, linkage to a cellular ligand or other protein, etc. A derivative of a polypeptide of the invention may be modified by chemical modifications using techniques known to those of skill in the art, including, but not limited to specific chemical cleavage, acetylation, formylation, metabolic synthesis of tunicamycin, etc. Furthermore, a derivative of a polypeptide of the invention may contain one or more non-classical amino acids.

In one embodiment, a polypeptide derivative is a functionally active derivative and possesses at least one, preferably more, similar or identical functions as a MOLL polypeptide of the invention such as, but not limited to, any one of the following: binding to antibodies that are raised against the wild type MOLL polypeptide, altering LDL levels, altering oxidized LDL levels, altering ROS levels, altering cholesterol levels, altering defecation cycle length of a C. elegans, and altering the developmental rate of a C. elegans organism (e.g., embryonic or post-embryonic development ) or a tissue thereof (e.g., germline tissue development). In another embodiment, a derivative of a polypeptide of the invention has an increased or decreased activity in one or more of the foregoing functions when compared to an unaltered polypeptide. Other altered activities include, but are not limited to, resistance to proteolysis or increased ability to cross a cell membrane.

In one embodiment, polypeptides that are at least 75%, 80%, 85%, 90%, 95%, 98%, or 99% identical to a MOLL polypeptide identified by the screening methods of the invention or variants thereof are encompassed by the invention. The degree of similarity (or percent identity) can be calculated by methods disclosed in Section 5.1.1 (with the caveat that NBLAST is not used in BLAST amino acid searches, rather XBLAST is used with program parameters set, e.g., to score-50, wordlength=3). In specific embodiments, a variant of DSC-4 comprises the consensus sequence as shown in FIG. 11 (SEQ ID NO:13), where for amino acid residue positions where no consensus residue exists, said positions consist of one of the amino acid residues found aligned at that residue position in any of the aligned human or C.elegans sequences shown in FIG. 11. In certain embodiments, a variant of DSC-4 comprises the consensus sequence as shown in FIG. 11 (SEQ ID NO:13), where for an amino acid residue position where no consensus residue exists, that position consists of an amino acid that is a conservative substitution of one of the amino acid residues found aligned at that residue position in any of the aligned human or C. elegans sequences shown in FIG. 11, with the proviso that the variant DSC-4 sequence does not comprise the aligned human sequences shown in FIG. 11.

In another embodiment, fragments of a MOLL polypeptide of the invention or variants thereof are encompassed by the invention. The invention features polypeptides which include a fragment of at least 5, 10, 15, 20, 25, 40, 50, 60, 70, 80, 90, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, or 1400 consecutive amino acid residues of the amino acid sequence of a MOLL polypeptide or variant thereof. A fragment of a polypeptide of the invention may or may not be immunogenic and/or antigenic. Preferably, a fragment of a polypeptide of the invention retains some level of function in at least one activity of the full length polypeptide. In one embodiment, the fragment comprises a one or more exons and/or domains (e.g., functional domains) of the MOLL polypeptide of the invention. In a specific embodiment, where the MOLL is dsc-4, the fragment comprises one or more of the apoB binding domain, the PDI binding domain, or the lipid binding domain (see e.g., FIGS. 3C and 4). In another embodiment, where the MOLL is dsc-3, the fragment comprises one or more of functional domains, such as the phosphorylation domain in the large cytoplasmic loop, the ATP binding domains, the ATP hydrolysis domain, or the hinge domain that connects the ATP binding sites to conserved residues within the transmembrane domains for substrate translocation.

Accordingly, in another embodiment, an isolated polypeptide of the invention is at least 25, 50, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, or 1400 contiguous amino acids in length and the nucleic acid that encodes such a polypeptide of the invention hybridizes under stringent conditions to the nucleic acid that encodes a MOLL polypeptide isolated by the methods of the invention or a variant thereof.

In addition to naturally-occurring allelic variants of a polypeptide of the invention, the skilled artisan will further appreciate that changes can be introduced by mutation into the nucleotide sequence of a nucleic acid encoding a polypeptide of the invention that may or may not result in changes in the biological activity of the protein. Such mutant polypeptides are also encompassed in the invention.

Accordingly, in another embodiment, the invention pertains to polypeptides that contain changes in amino acid residues that may or may not be essential for at least one activity. Such polypeptides differ in amino acid sequence from a MOLL polypeptide yet retain at least one biological activity of that MOLL polypeptide.

Another aspect of the invention pertains to polypeptides that are immunospecifically bound to by an antibody that immunospecifically binds to any one of the MOLL polypeptides identified by the screening methods of the invention.

In one embodiment, MOLL polypeptides can be used to make MOLL antibodies, preferably monoclonal antibodies. MOLL antibodies can be used as probes to monitor expression levels of MOLL gene products (or polypeptides). In a specific embodiment, MOLL polypeptide expression is used to diagnose disorders involving undesirable levels of lipids, bile salts/acids, lipoproteins (including but not limited to LDL) and/or ROS. In another specific embodiment, MOLL polypeptide expression is used to monitor effectiveness of treatment (either MOLL based or non- MOLL based treatment) of disorders involving undesirable levels of lipids, bile salts/acids, lipoproteins (including but not limited to LDL) and/or ROS. In another specific embodiment, MOLL polypeptide expression is used to predict those individuals predisposed or likely to suffer from a disorder involving undesirable levels of lipids, bile salts/acids, lipoproteins (including but not limited to LDL) and/or ROS.

In another embodiment, MOLL polypeptides are agents of the invention to be used in methods of treatment of disorders involving undesirable levels of LDL and/or ROS, or abnormal lipid or bile metabolism. For example, MOLL polypeptides can be administered to an individual to increase MOLL level in vivo.

In another embodiment, MOLL polypeptides can be used in drug screening assays to identify agents of the invention that bind to the MOLL polypeptide.

5.1.2.1 Antibodies

The present invention encompasses antibodies, or fragments thereof that immunospecifically bind to a MOLL polypeptide of the invention (e.g., DSC-3 or DSC-4). The antibodies of the invention can be polyclonal antibodies or monoclonal antibodies. The term “immunospecifically” as used herein refers to the ability of an antibody of the invention to bind a MOLL polypeptide without cross-reactivity with other non-MOLL polypeptides.

In one embodiment, the invention provides uses of substantially purified antibodies or fragments thereof, including human, non-human, or humanized antibodies or fragments thereof, which antibodies or fragments immunospecifically bind to a polypeptide of the invention comprising an amino acid sequence of SEQ ID NO:2 or 8 and an amino acid sequence which is encoded by the polynucleotide consisting of SEQ ID NO:1 or 7; or a fragment of at least 8 contiguous amino acid residues of the amino acid sequence of SEQ ID NO:2 or 8. Non-human antibodies can be goat, mouse, sheep, horse, chicken, rabbit, or rat antibodies.

In other embodiments, the invention provides substantially purified antibodies or fragments thereof, including human, non-human, or humanized antibodies or fragments thereof, which antibodies or fragments immunospecifically bind to a polypeptide of the invention comprising: i) a naturally occurring allelic variant of a polypeptide comprising the amino acid sequence of SEQ ID NO:2 or 8, wherein the polypeptide is encoded by a MOLL nucleic acid which hybridizes with a MOLL nucleic acid consisting of the nucleotide sequence of SEQ ID NO:1 or 7, or a complement thereof under stringent conditions; ii) a polypeptide that is encoded by a MOLL nucleic acid comprising a nucleotide sequence which is at least 90% identical to a nucleic acid consisting of SEQ ID NO:1 or 7, or a complement thereof; and iii) a polypeptide that is at least 90% identical to the amino acid sequence of SEQ ID NO:2 or 8. Non-human antibodies can be goat, mouse, sheep, horse, chicken, rabbit, or rat antibodies. The antibodies of the invention can be used in the methods of the invention.

In specific embodiments, the antibody of the invention binds to an exon or domain (e.g., functional domain) of a MOLL polypeptide of the invention, and prevents binding of the polypeptide to an endogenous binding partner, or causes the polypeptide to be degraded. In a more specific embodiment, where the MOLL is DSC-4, the domain is the apoB binding domain, the PDI binding domain, or the lipid binding domain (see e.g., FIG. 3C). In another specific embodiment, where the MOLL is DSC-3, the domain can comprise one or more of its transmembrane domain, (see FIG. 11).

In various embodiments, the antibodies of the present invention bind to the same epitope as any the antibodies that immunospecifically bind to polypeptides of the invention or competes with any of the antibodies that immunospecifically bind to polypeptides of the invention, e.g. as assayed by ELISA or any other appropriate immunoassay. As used herein, the term “epitope” refers to a portion of a polypeptide of the invention having antigenic or immunogenic activity in an animal, preferably in a mammal, and most preferably in a human. An epitope having immunogenic activity is a portion of a polypeptide of the invention that elicits an antibody response in an animal. An epitope having antigenic activity is a portion of a polypeptide of the invention to which an antibody immunospecifically binds as determined by any method well known in the art, for example, by immunoassays. Antigenic epitopes need not necessarily be immunogenic. An epitope can comprise post-translationally modified residues on the polypeptide, e.g., glycosylations and phosphorylations.

As used herein, the term “antibodies or fragments thereof that immunospecifically bind to a polypeptide of the invention” refers to antibodies or fragments thereof that specifically bind to a MOLL polypeptide of the invention (e.g., DSC-3 or DSC-4) and do not specifically bind to other polypeptides. Preferably, antibodies or fragments that immunospecifically bind to a polypeptide of the invention or a fragment thereof do not cross-react with other antigens. Antibodies or fragments that immunospecifically bind to a polypeptide of the invention can be identified, for example, by immunoassays or other techniques known to those of skill in the art. Antibodies of the invention include, but are not limited to, synthetic antibodies, monoclonal antibodies, recombinantly produced antibodies, multispecific antibodies, human antibodies, humanized antibodies, chimeric antibodies, single-chain Fvs (sFv), single chain antibodies, intrabodies, Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to an antigen of a polypeptide of the invention (e.g., one or more complementarity determining regions (CDRs) of an antibody). The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In various embodiments, the antibodies of the invention, or fragments thereof, can be chimeric and/or humanized antibodies. The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multispecific antibodies may immunospecifically bind to different epitopes of a MOLL polypeptide of the invention or may immunospecifically bind to a MOLL polypeptide of the invention as well a heterologous epitope, such as a heterologous polypeptide as described by Segal in U.S. Pat. No. 4,676,980. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies used in the methods of the invention include derivatives that are modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies of the invention or fragments thereof that comprise a framework region known to those of skill in the art. Preferably, the antibody of the invention or fragment thereof is human or humanized. In a specific embodiment, the antibody of the invention or fragment thereof comprises one or more CDRs from any antibody that immunospecifically binds a MOLL polypeptide of the invention. In a more specific embodiment, the antibody of the invention or fragment thereof comprises one or more CDRs from any antibody that immunospecifically recognizes a MOLL polypeptide of the invention.

The present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079). In one embodiment, the present invention provides single domain antibodies comprising two VH domains having modifications such that single domain antibodies are formed and having the amino acid sequence of any of the VH domains from any antibody that immunospecifically binds a MOLL polypeptide of the invention. In another embodiment, the present invention also provides single domain antibodies comprising two VH domains comprising one or more of the VH CDRs from any antibody that immunospecifically binds a MOLL polypeptide of the invention.

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication No. WO 97/34631). Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifuinctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The present invention also encompasses the use of antibodies or antibody fragments comprising the amino acid sequence of an antibody that immunospecifically binds a MOLL polypeptide of the invention with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays) can be used to assay the affinity of an antibody for a particular antigen.

Standard techniques known to those skilled in the art can be used to introduce mutations in the nucleotide sequence encoding an antibody, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original antibody or fragment thereof. In a preferred embodiment, the derivatives have conservative amino acid substitutions made at one or more predicted non-essential amino acid residues.

The antibodies of the invention or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” refers to an antibody that is derived from a single clone, including any eukaryotic, prokaryotic, or phage clone, and not the method by which it is produced (e.g., hybridoma technology).

The methods of the invention also encompass polynucleotides that encode or hybridize under high stringency, intermediate or lower stringency hybridization conditions to polynucleotides that encode an antibody of the invention.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., supra and Ausubel et al., eds., 1998, Current Protocols in Molecular Biology, John Wiley & Sons, NY), to generate antibodies having a different amino acid sequence, for example to create amino acid substitutions, deletions, and/or insertions. Other alterations to the polynucleotide are encompassed by the present invention and within the skill of the art.

5.1.2.1.1 Intrabodies

In certain embodiments, MOLL polypeptides of the invention are intracellular polypeptides. Thus, it may be advantageous for an antibody to bind the antigen intracellularly i.e., an intrabody. An intrabody comprises at least a portion of an antibody that is capable of immunospecifically binding an antigen and preferably does not contain sequences coding for its secretion. Such an intrabody can be used to modulate the activity of the polypeptide of the invention to which it binds. In one embodiment, an antagonistic intrabody is administered to decrease the activity of a polypeptide of the invention. In another embodiment, an agonistic intrabody is administered to increase the activity of a polypeptide of the invention. In another embodiment, an intrabody of the invention is administered such that it localizes to a specific subcellular compartment and thus modulates a polypeptide of the invention exclusively in that location.

In one embodiment, the intrabody comprises a single-chain Fv (“sFv”). sFvs are antibody fragments comprising the VH and VL domains of antibody, wherein these domains are present in a single polypeptide chain. Generally, the sFv polypeptide further comprises a polypeptide linker between the VH and VL domains which enables the sFv to form the desired structure for antigen binding. For a review of sFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, WA, 1998, “Intrabodies: Basic Research and Clinical Gene Therapy Applications” Springer:New York).

Generation of intrabodies is well-known to the skilled artisan and is described, for example, in U.S. Pat. Nos. 6,004,940; 6,072,036; 5,965,371. Further, the construction of intrabodies is discussed in Ohage and Steipe, 1999, J Mol. Biol. 291:1119-1128; Ohage et al., 1999, J. Mol. Biol. 291:1129-1134; and Wirtz and Steipe, 1999, Protein Science 8:2245-2250. Recombinant molecular biological techniques such as those described for recombinant production of antibodies (e.g., Sections 5.1.2.1 and 5.2) may also be used in the generation of intrabodies.

In one embodiment, the recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well. Examples of membrane permeable sequences are the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, von Heijne, 1987, Prot. Seq. Data Anal. 1:41-2; von Heijne and Abrahmsen, 1989, FEBS Lett. 224:439-46

In another embodiment, a polynucleotide encoding an intrabody is administered to a patient (e.g., as in gene therapy). In this embodiment, methods as described in Section 5.6.2 can be used to administer the intrabody polynucleotide.

### 5.2 Recombinant Expression

Another aspect of the invention pertains to vectors, preferably expression vectors, comprising a nucleic acid of the invention, or a variant thereof. As used herein, the term “vector” refers to a polynucleotide capable of transporting another nucleic acid to which it has been linked. One type of vector is a “plasmid”, which refers to a circular double stranded DNA loop into which additional DNA segments can be introduced. Another type of vector is a viral vector, wherein additional DNA segments can be introduced into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses).

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably associated with the polynucleotide to be expressed. Within a recombinant expression vector, “operably associated” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology, (1990) Academic Press, San Diego, CA, p. 185. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a MOLL polypeptide of the invention in prokaryotic (e.g., E. coli) or eukaryotic cells (e.g., insect cells using baculovirus expression vectors, yeast cells, C. elegans cells, or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors comprising constitutive or inducible promoters directing the expression of either fusion or non-fusion proteins. Fusion vectors add a number of amino acids to a protein encoded therein, usually to the amino terminus of the recombinant protein. Such fusion vectors typically serve at least three purposes: 1) to increase expression of recombinant protein; 2) to increase the solubility of the recombinant protein; and/or 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, MA) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., Gene Expression Technology: Methods in Enzymology 185, Academic Press, San Diego, Calif. (1990) p. 60-89). Target gene expression from the pTrc vector relies on host RNA polymerase transcription from a hybrid trp-lac fusion promoter. Target gene expression from the pET 11d vector relies on transcription from a T7 gn 10-lac fusion promoter mediated by a coexpressed viral RNA polymerase (T7 gn1). This viral polymerase is supplied by host strains BL21(DE3) or HMS174(DE3) from a resident Xprophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) p. 119-128). Another strategy is to alter the sequence of the nucleic acid to be inserted into an expression vector so that the individual codons for each amino acid are those preferentially utilized in E. coli (Wada et al., 1992, Nucleic Acids Res. 20:2111-2118). Such alteration of polynucleotides of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corp., San Diego, Calif.), and pPicZ (Invitrogen Corp., San Diego, Calif.).

In another embodiment, the expression vector is a baculovirus expression vector. Baculovirus vectors available for expression of proteins in cultured insect cells (e.g., Sf9 cells) include the pAc series (Smith et al., 1983, Mol. Cell Biol. 3:2156-2165) and the pVL series (Lucklow and Summers, 1989, Virology 170:31-39).

In another embodiment, the expression vector is a C. elegans expression vector. Examples of vectors for expression in C. elegans include a whole set of publicly available vectors from the Fire vector collection (http://ftp.ciwemb.edu/PNF:byName:/FireLabWeb/FireLablnfo/FireLabVectors/) including vectors with heat shock promoters such as pPD49.78 and pPD49.83, and promoterless vectors in which sequence of the gene of interest, including its upstream regulatory region, is cloned in frame with the gene fluorescent protein gene to monitor expression by epifluorescence (see generally Mello and Fire, 1995, DNA transformation in “Methods in Cell Biology Caenorhabditis elegans: Modern Biological Analysis of an organism” vol. 48, Shakes and Epstein eds., Academic Press:San Diego). Additionally, gene expression can be directed in C. elegans by injection of cDNA with its promoter region (e.g. a PCR product or restriction fragment) not cloned into any vector.

In yet another embodiment, a nucleic acid of the invention is expressed in mammalian cells using a mammalian expression vector. Examples of mammalian expression vectors include pCDM8 (Seed, 1987, Nature 329:840-842) and pMT2PC (Kaufmnan et al., 1987, EMBO J. 6:187-193). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al. 1990, Molecular Cloning, A Laboratory Manual, 2d Ed., Cold Spring Harbor Laboratory, Cold Spring Harbor, N.Y.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banedji et al., 1983, Cell 33:729-740; Queen and Baltimore, 1983, Cell 33:741-748), neuron-specific promoters (e.g., the neurofilament promoter; Byrne and Ruddle, 1989, PNAS 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Campes and Tilghman, 1989, Genes Dev. 3:537-546).

The invention further provides a recombinant expression vector comprising a polynucleotide of the invention cloned into the expression vector in an antisense orientation. That is, the DNA molecule is operably associated with a regulatory sequence in a manner which allows for expression (by transcription of the DNA molecule) of an RNA molecule which is antisense to the mRNA encoding a polypeptide of the invention. Regulatory sequences operably associated with a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

In another embodiment, the expression characteristics of an endogenous gene corresponding to a nucleic acid of the invention within a cell, cell line or microorganism may be modified by inserting a DNA regulatory element heterologous to the endogenous gene of interest into the genome of a cell, stable cell line or cloned microorganism such that the inserted regulatory element is operatively linked with an endogenous gene and controls, modulates or activates the endogenous gene. For example, endogenous genes of the invention which are normally “transcriptionally silent”, i.e., genes which are normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous genes of the invention may be activated by insertion of a promiscuous regulatory element that works across cell types.

A heterologous regulatory element may be inserted into a stable cell line or cloned microorganism, such that it is operatively linked with and activates expression of an endogenous gene corresponding to a nucleic acid of the invention, using techniques, such as targeted homologous recombination, which are well known to those of skill in the art (See, e.g., U.S. Pat. Nos. 5,272,071 and 5,968,502; International Publication Nos. WO 91/06667 and WO 94/12650). Alternatively, non-targeted techniques (e.g., non-homologous recombination) well known in the art can be used (see, e.g., International Publication No. WO 99/15650).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Accordingly, the present invention provides a host cell having an expression vector comprising a nucleic acid of the invention, or a variant thereof. A host cell can be any prokaryotic (e.g., E. coli) or eukaryotic cell (e.g., insect cells, yeast or mammalian cells). The invention also provides a method for expressing a nucleic acid of the invention thus making the encoded polypeptide (e.g., MOLL polypeptide such as DSC-3 and DSC-4) comprising the steps of (a) culturing a cell comprising a recombinant nucleic acid of the invention under conditions that allow said polypeptide to be expressed by said cell; and isolating the expressed polypeptide.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide of the invention from the medium or the host cell.

### 5.3 Preparation of C. elegans for Assays of the Invention

In one embodiment, the C. elegans used in the screening assays of the invention have at least one mutation. The mutation affects the level of cholesterol, LDL and/or ROS such that the C. elegans displays one or more of the phenotypes associated with undesirable levels of LDL and/or ROS (e.g., increased or decreased defecation cycle length, heterochronic germline development, increased or decreased rate of embryonic or post-embryonic development). The mutation may be one that is naturally occurring, has been induced randomly, or has been introduced by site directed mutagenesis or RNAi, etc.

In one embodiment, the C. elegans used in the screening assays of the invention contains a mutation in clk-1. Wild type clk-1 sequences can be found e.g., in Genbank as Accession Nos. NM_{—}065727, NM_{—}009940, and NM_{—}06138. Any method of mutagenesis known in the art (e.g., those described below) can be used to make mutant clk-1 for use in the assays of the invention. In a specific embodiment, the C. elegans used for screening has a clk-1(qm30) mutation. In another specific embodiment, the C. elegans used for screening has a clk-1 (e2519) mutation. In another specific embodiment, the C. elegans used for screening has a clk-1(qm51) mutation. (see generally International Patent Publication No. WO 98/17823, Felkai et al., 1999, EMBO J 18: 1783-1792, Wong et al., 1995, Genetics 139: 1247-1259, Ewbank et al., 1997, Science 275:980-983, Branicky et al., 2001, Genetics 159:997-1006 for clk-1 mutations). In another embodiment, mutations that have been isolated in clk-1 homologues in other species can be used in the assays of the invention. Corresponding mutations can be made in the C. elegans clk-1 through directed mutagenesis. For example, Marbois and Clark (1996, J. Biol. Chem. 271:2995-3004) describe a mutation in coq-7, the yeast clk-1 homologue.

In another embodiment, the C. elegans used in the screening assays of the invention contains a mutation in a MOLL. In a specific embodiment, the MOLL is selected from the group consisting of dsc-1, dsc-2, dsc-3, dsc-4, dsc-5, and dsc-7. Any method of mutagenesis known in the art (e.g., those described below) can be used to make mutant MOLL for use in the assays of the invention. In a more specific embodiment, the MOLL is dsc-4. In another specific embodiment, the MOLL is dsc-3. Wild type sequence of dsc-4 and dsc-3 is SEQ ID NO:1, and SEQ ID NO:7 respectively. In an even more specific embodiment, the C. elegans used for screening has a dsc-4(qm 182) mutation.

5.3.1 For Target Identification

In one embodiment, the invention encompasses the use of C. elegans to identify target genes involved in alteration of the level of a lipid or a lipoprotein, such as cholesterol and LDL, as well as ROS. In such an embodiment, a C. elegans having a mutant clk-1 or mutant MOLL (e.g., dsc-3 or dsc-4) such that it displays a phenotype associated with undesirable levels of cholesterol, LDL and/or ROS (e.g., increased or decreased defecation cycle length, heterochronic germline development, increased or decreased rate of embryonic or post-embryonic development; see Section 5.4.2) is further mutagenized. Worms are then scored for either a lessening of the mutant phenotype (i.e., a phenotype that is more similar to wild type than the initial mutant phenotype) or a worsening of the mutant phenotype (i.e., a phenotype that is even less similar to wild type than the initial mutant phenotype).

Any method known in the art can be used for generating mutants to be used in the target screening assays of the invention. Methods of mutagenesis can be used which randomly generate mutations in the genome of the C. elegans including, but not limited to, EMS chemical deletion mutagenesis and Tc1 transposon insertion mutagenesis. Alternatively, methods of mutagenesis can be used which are directed to a particular gene (e.g., a gene that is expected or predicted to be a MOLL) including, but not limited to, RNAi and molecular evolution techniques such as site directed mutagenesis. In addition to its use in directed mutagenesis, RNAi mutagenesis may also be used to identify heretofore unknown MOLLs. The expression of each gene in C. elegans can be systematically decreased/inhibited using RNAi high throughput techniques (see, e.g., Kamath et al., 2003, Nature 421:231-7, Ashrafi et al., 2003, Nature 421:268-72, Tasch1, 2003, Nature 421:220-221 and Section 5.3.1.4). Because this technique can be used without prior identification of candidate MOLLs, RNAi can be used in the same way as random mutagenesis methods.

Mutations that rescue clk-1 mutant C. elegans by decreasing native LDL levels can fall into two main classes—namely those mutations which decrease native LDL synthesis/secretion and those mutations which promote the conversion of native LDL to oxidized LDL. The genes identified in the screen in each category of clk-1 suppressors can be heterogeneous in their normal functions yet all share the characteristic of decreasing native LDL levels when mutated. A further degree of heterogeneity is introduced due to the fact that mutagenesis can affect the function of a gene in different ways (e.g., complete or partial loss of function, gain of function, overexpression, etc.). Thus, the function of a gene can either be decreased or enhanced depending upon the type of mutation present.

The members of the class of genes identified as clk-1 suppressors that decrease native LDL synthesis/secretion when mutated may have a variety of different functions when not mutant. For example, those genes which have been mutated such that their normal function is decreased can have a normal function involved in i) increasing LDL synthesis/secretion, ii) promoting the expression or activity of a molecule involved in increasing LDL synthesis/secretion, iii) decreasing the expression or activity of a molecule involved in inhibiting LDL synthesis/secretion, iv) increasing lipid levels generally such that more are available for incorporation into LDL, or v) promoting the expression or activity of a molecule involved in increasing lipid levels generally, or vi) decreasing the expression or activity of a molecule involved in lowering lipid levels generally. Alternatively, those genes which have been mutated such that their normal function is enhanced can have a normal function involved in i) decreasing LDL synthesis/secretion, ii) promoting the expression or activity of a molecule involved in decreasing LDL synthesis/secretion, iii) increasing the expression or activity of a molecule involved in inhibiting LDL synthesis/secretion, iv) decreasing lipid levels generally such that less are available for incorporation into LDL, v) promoting the expression or activity of a molecule involved in decreasing lipid levels generally or vi) decreasing the expression or activity of a molecule involved in increasing lipid levels generally.

Similarly, the members of the class of genes identified as clk-1 suppressors that promote the conversion of native LDL to oxidized LDL when mutated may have a variety of different functions when not mutated. For example, those genes which have been mutated such that their normal function is decreased can have a normal function involved in i) decreasing LDL oxidation, ii) promoting the expression or activity of a molecule involved in decreasing LDL oxidation, iii) decreasing the expression or activity of a molecule involved in increasing LDL oxidation, iv) decreasing ROS production, v) promoting the expression or activity of a molecule involved in decreasing ROS production, vi) decreasing the expression or activity of a molecule involved in increasing ROS production, vii) increasing ROS clearance, viii) promoting the expression or activity of a molecule involved in increasing ROS clearance, or ix) decreasing the expression or activity of a molecule involved in decreasing ROS clearance. Alternatively, those genes which have been mutated such that their normal function is enhanced can have a normal function involved in i) increasing LDL oxidation, ii) promoting the expression or activity of a molecule involved in increasing LDL oxidation, iii) decreasing the expression or activity of a molecule involved in decreasing LDL oxidation, iv) increasing ROS production, v) promoting the expression or activity of a molecule involved in increasing ROS production, vi) decreasing the expression or activity of a molecule involved in decreasing ROS production, vii) decreasing ROS clearance, viii) promoting the expression or activity of a molecule involved in decreasing ROS clearance, or ix) decreasing the expression or activity of a molecule involved in increasing ROS clearance.

Although each of these genes identified as clk-1 suppressors can have very different functions from each other, they each can functionally affect native LDL levels. Because of this common characteristic, each can be used as a target for drug discovery such that compounds are identified which affect native LDL levels (see Section 5.3.2).

Preferably, a mutant MOLL polypeptide exhibits altered activity in at least one function displayed by the wild type MOLL polypeptide. The altered activity of the mutant polypeptide can be a decrease (e.g., loss-of-function mutation) or increase (e.g., gain-of-function mutation) in activity. As used herein, the phrase “loss-of-function mutation” refers to a mutation such that the mutant polypeptide has decreased activity. The decreased activity may be present in each of the functions/activities of the polypeptide or may present in fewer than all of the functions/activities of the polypeptide. A loss-of-function mutation can be a complete (null) or partial loss-of-function. As used herein, the phrase “gain-of-function mutation” refers to a mutation such that the mutant polypeptide has increased activity. The increased activity may be present in each of the functions/activities of the polypeptide or may be present in fewer than all of the functions/activities of the polypeptide.

5.3.1.1 EMS Chemical Deletion Mutagenesis

Ethyl methanesulfonate (EMS) is a commonly-used chemical mutagen for creating loss-of-function mutations in genes-of-interest in C. elegans. Approximately 13% of mutations induced by EMS are small deletions. With the methods described herein, there is approximately a 95% probability of identifying a deletion-of-interest by screening 4×106 EMS-mutagenized genomes. After mutagenesis, mutant C. elegans are further screened to identify those mutations that are in a gene encoding a polypeptide of the invention. Briefly, this procedure involves creating a library of several million mutagenized C. elegans which are distributed in small pools in 96-well plates, each pool composed of approximately 400 haploid genomes. A portion of each pool is used to generate a corresponding library of genomic DNA derived from the mutagenized nematodes. The DNA library is screened with a PCR assay to identify pools that carry genomes with deletions-of-interest, and mutant worms carrying the desired deletions are recovered from the corresponding pools of the mutagenized animals. Although EMS is a preferred mutagen to generate deletions, other mutagens can be used that also provide a significant yield of deletions, such as X-rays, gamma-rays, diepoxybutane, formaldehyde and trimethylpsoralen with ultraviolet light.

Nematodes may be mutagenized with EMS using any procedure known to one skilled in the art, such as the procedure described by Sulston and Hodgkin (1988, pp. 587-606, in The Nematode Caenorhabditis elegans, Wood, Ed., Cold Spring Harbor Laboratory Press, Cold Spring Harbor, N.Y.). Following exposure to the mutagen, nematodes are dispensed into petri dishes, incubated one to two days, and embryos isolated by hypochlorite treatment (Id.) Embryos are allowed to hatch and L1 larvae are collected following overnight incubation. The larvae are distributed in petri plates at an average density of 200 animals per plate and incubated for 5 to 7 days until just starved. A sample of nematodes is collected from each plate by washing with a solution of distilled water, and the nematodes washed from each plate are placed in one well of a 96-well plate. Worms are lysed and DNA stored at −80° C. until further analysis. Live nematodes from each plate are aliquoted into tubes within racks for storage at −80° C., such that the physical arrangement of tubes of live animals is the same as the arrangement of corresponding DNA lysates in the 96-well plates.

A pooling strategy is used to allow efficient PCR screening of the DNA lysates. The pools are made from each 96-well plate by mixing 10 μl of lysate from 8 wells comprising each column of wells in a plate. The pooled lysates for each column are used for screening with PCR. PCR primers are designed for each locus-of-interest to be about 1.5 to 12 kb apart, depending on the size of the locus, such that deletions encompassing the entire coding regions of MOLL nucleic acids of the invention can be detected following a previously-described procedure (see Plasterk, 1995, Methods in Cell Biology 48:59-80). For each region, two sets of primer pairs are chosen for carrying out a nested PCR strategy such that an outside set is used for the first round of PCR and an inside set is used for the second round of PCR. The second round of PCR is performed to achieve greater specificity in the reaction.

Products of the second round of PCR may be analyzed by electrophoresis in agarose or acrylamide gels. If a potential deletion product is observed in at least one of the two reactions, two rounds of PCR are performed as described above on lysates from each individual well derived from the column corresponding to the positive pool. This results in the identification of a positive “address,” i.e., a specific well within an individual plate, containing a deletion mutant. The positive address is re-tested in quadruplicate using two rounds of PCR as described above, and the product is gel purified and sequenced directly to confirm the presence of the desired deletion.

Once a positive address has been identified and confirmed by sequence analysis, approximately 300 individual worms from the relevant plate are cloned onto separate, fresh plates. When F1 animals are present on the plate, the parent nematodes are placed into buffer and lysed as described above. The same primer pairs and cycling conditions used to identify the deletion are used to perform PCR on these animals. Once a single animal carrying the deletion has been identified, its progeny are cloned and examined using the same conditions described above, until a homozygous population of deletion animals is obtained.

5.3.1.2 Tc1 Transposon Insertion Mutagenesis

The transposable element Tc1 may also be used as a mutagen in C. elegans since insertion of the transposable element into a gene-of-interest can result in the inactivation of gene function. After mutagenesis, mutant C. elegans are further screened to identify those mutations that are in a gene encoding a polypeptide of the invention. Starting with a strain that contains a high copy number of the Tc1 transposable element in a mutator background (i.e., a strain in which the transposable element is highly mobile), a Tc1 library containing approximately 3,000 individual cultures is created as previously described (see e.g., Zwaal et al., 1993, PNAS 90:7431-7435; Plasterk, 1995, “Reverse Genetics: From Gene Sequence to Mutant Worm”, in Caenorhabditis elegans: Modem Biological Analysis of an Organism (Epstein and Shakes, Eds.) pp. 59-80.). The library is screened for Tc1 insertions in the region of interest using the polymerase chain reaction with one set of primers specific for Tc1 sequence and one set of gene-specific primers (e.g., primers for clk-2). Because Tc1 exhibits a preference for insertion within introns, it is sometimes necessary to carry out a secondary screen of populations of insertion animals for imprecise excision of the transposable element, which can result in deletion of part or all of the gene of interest (generally, 1-2 kb of genomic sequence is deleted). The screen for Tc1 deletions is performed and deletion animals are recovered in the same manner as for the EMS screen described above.

5.3.1.3 Molecular Evolution Techniques

Mutant polypeptides can be created by introducing one or more nucleotide substitutions, additions or deletions into the nucleotide sequence of the nucleic acids of the invention (e.g., dsc-4), such that one or more amino acid substitutions, additions or deletions are introduced into the encoded protein. Mutations can be introduced by standard techniques such as directed molecular evolution techniques (see generally Arnold, 1993, Curr. Opinion Biotechnol. 4:450-455); e.g., site-directed mutagenesis (see e.g., Kunkel, 1985, PNAS, 82:488-492; Oliphant et al., 1986, Gene 44:177-183); oligonucleotide-directed mutagenesis (see e.g., Reidhaar-Olson et al., 1988, Science 241:53-57); chemical mutagenesis (see e.g., Eckert et al., 1987, Mutat. Res. 178:1- 10); error prone PCR (see e.g., Caldwell & Joyce, 1992, PCR Methods Applic. 2:28-33); cassette mutagenesis (see e.g., Arkin et al., PNAS, 1992, 89:7871-7815); DNA shuffling methods (see e.g., Stemmer et al., 1994, PNAS, 91:10747-10751; U.S. Pat. Nos. 5,605,793; 6,117,679; 6,132,970; 5,939,250; 5,965,408; 6,171,820).

In one embodiment, particular nucleotide sequences or positions of a nucleic acid are targeted for mutation. Such targeted mutations can be introduced at any position in the nucleic acid. For example, one can make nucleotide substitutions leading to amino acid substitutions at “non-essential” or “essential” amino acid residues. A “non-essential” amino acid residue is a residue that can be altered from the wild-type sequence without altering the biological activity, whereas an “essential” amino acid residue is required for at least one biological activity of the polypeptide. For example, amino acid residues that are not conserved or only semi-conserved among homologs of various species may be non-essential for activity. Alternatively, amino acid residues that are conserved among the homologs of various species (e.g., mouse and human) may be essential for activity.

Such targeted mutations can also be made at one or more non-conservative amino acid residues. A “non-conservative amino acid substitution” is one in which the amino acid residue is replaced with an amino acid residue having a dissimilar side chain. Families of amino acid residues having similar side chains have been defined in the art. These families include amino acids with basic side chains (e.g., lysine, arginine, histidine), acidic side chains (e.g., aspartic acid, glutamic acid, asparagine, glutamine), uncharged polar side chains (e.g., glycine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

Alternatively or in addition to non-conservative amino acid residue substitutions, such targeted mutations are made at one or more conservative amino acid residues. A “conservative amino acid substitution” is one in which the amino acid residue is replaced with an amino acid residue having a similar side chain. Following mutagenesis, the encoded protein can be expressed recombinantly and the activity of the protein can be determined.

In another embodiment, mutations can be introduced randomly along all or part of the coding sequence (e.g., by saturation mutagenesis). In certain embodiments, nucleotide sequences encoding other related polypeptides that have similar domains, structural motifs, active sites, or that aligns with a portion of the enzyme gene of the invention with mismatches or imperfect matches, can be used in the mutagenesis process to generate diversity of sequences. It should be understood that for each mutagenesis step in some of the techniques mentioned above, a number of iterative cycles of any or all of the steps may be performed to optimize the diversity of sequences. The above-described methods can be used in combination in any desired order. In many instances, the methods result in a pool of mutant nucleotide sequences or a pool of recombinant host cells comprising mutant nucleotide sequences. The nucleotide sequences or host cells expressing a modified enzyme with the desired characteristics can be identified by screening with one or more assays that are well known in the art. The assays may be carried out under conditions that select for polypeptides possessing the desired physical or chemical characteristics. The mutations in the nucleotide sequence can be determined by sequencing the nucleic acid encoding the mutant polypeptide in the clones.

5.3.1.4 RNA Interference

In certain embodiments, an RNA interference (RNAi) molecule is used to mutagenize a C. elegans by decreasing or inhibiting expression of the nucleic acid against which the RNAi is directed. RNAi refers to the use of double-stranded RNA (dsRNA) or small interfering RNA (siRNA) to suppress the expression of a gene comprising a related nucleotide sequence. RNAi is also called post-transcriptional gene silencing (or PTGS). Since the only RNA molecules normally found in the cytoplasm of a cell are molecules of single-stranded mRNA, the cell has enzymes that recognize and cut dsRNA into fragments containing 21-25 base pairs (approximately two turns of a double helix and which are referred to as small interfering RNA or siRNA). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in many organisms including, but not limited to, C. elegans, mammals, etc. dsRNA is used as inhibitory RNA or RNAi of the function of a nucleic acid molecule of the invention to produce a phenotype that is the same as that of a null mutant of a nucleic acid molecule of the invention (Wianny & Zernicka-Goetz, 2000, Nature Cell Biology 2: 70-75).

Alternatively, siRNA can be introduced directly into a cell to mediate RNA interference (Elbashir et al., 2001, Nature 411:494-498). Many methods have been developed to make siRNA, e.g., chemical synthesis or in vitro transcription. Once made, the siRNAs are introduced into cells via transient transfection. See also U.S. patent applications Ser. No. 60/265232, 09/821832 and PCT/US01/10188, directed to RNA Sequence-Specific Mediators of RNA Interference. A number of expression vectors have also been developed to continually express siRNAs in transiently and stably transfected mammalian cells (Brummelkamp et al., 2002 Science 296:550-553; Sui et al., 2002, PNAS 99(6):5515-5520; Paul et al., 2002, Nature Biotechnol. 20:505-508). Some of these vectors have been engineered to express small hairpin RNAs (shRNAs), which get processed in vivo into siRNA-like molecules capable of carrying out gene-specific silencing. Another type of siRNA expression vector encodes the sense and antisense siRNA strands under control of separate pol III promoters (Miyagishi and Taira, 2002, Nature Biotechnol. 20:497-500). The siRNA strands from this vector, like the shRNAs of the other vectors, have 5′ thymidine termination signals. Silencing efficacy by both types of expression vectors was comparable to that induced by transiently transfecting siRNA.

RNAi technology has been adapted for high throughput use in C. elegans (see, e.g., Kamath et al., 2003, Nature 421:231-7, Ashrafi et al., 2003, Nature 421:268-72, Tasch1, 2003, Nature 421:220-221). Briefly, DNA plasmids encoding a double-stranded RNA (dsRNA) of choice are inserted into E. coli. The nucleic acid encoding the dsRNA can be placed under the control of an inducible promoter such that expression in E. coli occurs only in the presence of the inducing molecule (e.g., IPTG). Nematodes at the latest larval stage are placed on a lawn of E. coli expressing the dsRNA and allowed to feed on the E. coli. The ingested bacteria release the dsRNA inside the nematode. As a result, the gene whose sequence corresponds to that of the dsRNA behaves as if the gene carries a loss-of-function mutation.

5.3.2 For Agent Identification

In one embodiment, the invention encompasses the use of C. elegans to assay, screen for, or identify agents of the invention which alter the level of a lipid or a lipoprotein in an organism. In such an embodiment, a C. elegans comprising a mutant clk-1 and/or a mutant MOLL gene (e.g., dsc-3 or dsc-4) such that it displays a phenotype associated with undesirable levels of a lipid or a lipoprotein (e.g., increased or decreased defecation cycle length, heterochronic germline development, increased or decreased rate of embryonic or post-embryonic development; see Section 5.4) is incubated with or fed candidate compounds. Worms are then scored for either a lessening of the mutant phenotype (i.e., a phenotype that is more similar to wild type than the initial mutant phenotype) or a worsening of the mutant phenotype (i.e., a phenotype that is even less similar to wild type than the initial mutant phenotype).

Although not intending to be bound by a particular mechanism of action, an agent or compound of the invention can alter activity of a clk-1 or MOLL polypeptide by, e.g., enhancing/interfering with clk-1 or MOLL nucleic acid/polypeptide expression, enhancing or interfering with the clk-1 or MOLL polypeptide interaction with an endogenous binding partner (e.g., a polypeptide, lipid, or nucleic acid that interacts with the polypeptide in a wild type organism), enhancing/interfering with clk-1 or MOLL activity, etc. The methods generally involve incubating agents with animals that express a clk-1 or MOLL nucleic acid/polypeptide molecule (either mutant or wild type) and then assaying for an alteration in phenotype that is associated with undesirable levels of a lipid or a lipoprotein (e.g., defecation cycle length, heterochronic germline development, rate of embryonic or post-embryonic development) thereby identifying an agent of the invention. The invention also encompasses the use of biochemical assays to identify test compounds that bind to a clk-1 or MOLL polypeptide molecule. Compounds found to bind to a clk-1 or MOLL polypeptide can then be assayed in C. elegans-based assays to determine any phenotype-altering properties.

In one specific embodiment, the invention provides the use of C. elegans clk-1 mutant test nematodes to assay for compounds that can partially or completely restore the wild type phenotype, or that can phenocopy the presence of a dsc mutant (e.g., dsc-3 or dsc-4) or RNAi of a dsc gene in the clk-1 mutant nematode. The invention also provides the use of test nematodes comprising mutations in multiple genes (such as dobule mutants clk-1/dsc-3 and clk-1/dsc-4), or RNAi of one or more genes, to further characterize positive compounds that restore partially or completely a wild type phenotype(s) in a clk-1 mutant assay as described above. Because many of the phenotypes are quantitative traits, the methods allow the determination of whether the effect on the phenotype(s) is additive with respect to contact with a compound and the presence of the mutant genotypes. Such methods can be used to determine whether a positive compound is affecting the same process as one of the mutant genotypes.

In particular, agents or compounds that partially or completely rescue clk-1 mutant C. elegans by decreasing native LDL levels can fall into three main categories—namely those compounds which decrease cholesterol absorption, decrease native LDL synthesis/secretion and those compounds which promote the conversion of native LDL to oxidized LDL. The compounds in each category can be heterogeneous in nature but all share the characteristic of the ability to decrease native LDL levels. For example, compounds that decrease cholesterol absorption can act by decreasing the level or activity of a molecule involved in (i) binding of cholesterol, (ii) transport of cholesterol across plasma and/or organelle membranes, or (iii) conversion of cholesterol into a related sterol that can be absorbed and transported. Compounds that decrease native LDL synthesis/secretion can act by i) decreasing the expression or activity of a molecule involved in LDL synthesis/secretion, ii) promoting the expression or activity of a molecule involved in inhibiting LDL synthesis/secretion, iii) promoting the expression or activity of a molecule involved in lowering lipid levels generally such that they are unavailable for incorporation into LDL, or iv) decreasing the expression or activity of a molecule involved in raising lipid levels. Compounds that promote the conversion of native LDL to oxidized LDL can act by i) promoting the expression or activity of a molecule involved in LDL oxidation, ii) decreasing the expression or activity of a molecule involved in inhibiting LDL oxidation, iii) promoting the expression or activity of a molecule involved in ROS production, iv) decreasing the expression or activity of a molecule involved in inhibiting ROS production., v) decreasing the expression or activity of a molecule involved in increasing ROS clearance, or vi) promoting the expression or activity of a molecule involved in decreasing ROS clearance.

5.3.2.1 Candidate Agents

As used herein, the term “agent” refers to a molecule that has a desired biological effect. Agents include, but are not limited to, proteinaceous molecules, including, but not limited to, peptide, polypeptide, protein, post-translationally modified protein, antibodies etc.; or a large molecule, including, but not limited to, inorganic or organic compounds; or a small molecule (less than 500 daltons), including, but not limited to, inorganic or organic compounds; or a nucleic acid molecule, including, but not limited to, double-stranded DNA, single-stranded DNA, double-stranded RNA, single-stranded RNA, or triple helix nucleic acid molecules. Agents can be natural products derived from any known organism (including, but not limited to, animals, plants, bacteria, fungi, protista, or viruses) or from a library of synthetic molecules. As used herein, the terms “agent” and “compound” are used interchangeably.

In preferred embodiments, candidate agents can partially or completely restore one of the phenotypes of a clk-1 mutant nematode to wild type. In related embodiments, candidate agents can phenocopy one or more effects of a mutation in a dsc gene in a clk-1 mutant nematode. In specific embodiments, the candidate agents are antagonists of dsc gene products, such as but not limited to DSC-3 and DSC-4.

Examples of candidate agents are disclosed in U.S. Pat. Nos. 5,474,991, 5,929,091; 6,147,214, 6,197,798, 6,417,367, 6,444,664. Examples of candidate agents include specific and non-specific ATPase inhibitors, such as vanadates that modulate the enzymatic function of DSC-3, and drugs such as Ezetimibe (Zetia Schering Plough) that affect the common metabolic process. In certain embodiments of the methods of the invention other agents known to increase bile synthesis can be used as agents, such as but not limited to, taurine which increases the activity of the CYP7A1 enzyme.

### 5.4 Assays of the Invention

In the present invention, nematodes are used as a model for investigating the role of various genes in lipid metabolism. The inventors observed that nematodes that harbor mutations in the genes of the invention exhibit a variety of morphologic, behavioral, developmental phenotypes. RNA interference or incubation with agents of the invention as well as environmental factors, such as temperature can further modify such phenotypes. The model is based on the discovery that specific morphologic, behavioral, developmental phenotypes of the nematodes are associated with certain metabolic events or states which also occur in humans and are associated with human disorders.

In one embodiment, the nematode model can be used to identify genes that are involved in lipid metabolism including mechanisms that lead to pathogenesis of dyslipidemia, atherosclerosis and cardiovascular diseases. This can be accomplished by using a nematode that produces a specific phenotype, generating mutations in the genome of such nematodes, screening for mutant nematodes that display a modified phenotype, and isolating the gene that was mutated which produced the modified phenotype. For example, the invention provides that reducing cholesterol level, the production or secretion of LDL or the oxidation of LDL increase the rate of germline development. By identifying mutants that produce the same change in germ line development, the genes that play a role in the processes of germline development can be found. Accordingly, the invention provides assays in which nematodes of a specific genotype and phenotype is subjected to mutagenesis, and mutated nematodes that display a change in the phenotypes are isolated. Optionally, the mutated nematodes can be subjected to certain environment, such as a shift in temperature, to further distinguish and characterize the change in the phenotype.

In another embodiment, instead of generating random or non-random mutations in the nematode genome, the mRNA levels of many known or predicted genes in the nematode can be specifically reduced to test for a change in the phenotype. This can be accomplished by many methods known in the art, including RNA-mediated interference.

In another embodiment, the nematode model can be used for drug screening whereby the effect of a candidate compound can be assessed by observing changes in the phenotypes of a nematode which reflect changes in the disorder-related metabolic events or states, or correlate with a change in the activity of a known drug target. By using a nematode that produces a specific phenotype, the display of a modified phenotype after contact with a candidate compound indicates that the compound can affect the relevant metabolic events or states in the animal (e.g., an increase or decrease in the levels of certain lipids and/or lipoproteins), and interact with if known the respective drug target (e.g., MOLL polypeptides). Accordingly, the invention provides assays in which nematodes of a specific phenotype is contacted with a test compound, and nematodes that display a change in the phenotype after the contact are identified and isolated. The change in phenotype can be correlated to a change in the relevant metabolic events or states in the animal such an increase or decrease in the levels of certain lipids (e.g., cholsterol) and/or lipoproteins (e.g., LDL-like lipoprotein) at certain locations within the animal. The test compound that produced the change is then further analyzed for its mode of action, such as the target with which it interacted in the test nematode; and further developed as a drug candidate. Optionally, the nematodes can be subjected to certain environment, such as a shift in temperature, before, during, or after the contacting step to further distinguish and characterize the change in the phenotype for correlation with a certain level of lipid or lipoprotein, or a certain activity.

In yet another embodiments, genes from other organisms that can alter a particular phenotype of nematodes can be used in the screening assays of the invention. For example, the MOLL homologs from other organisms, or mutants thereof, including, but not limited to, the human genes ATP8B 1, ATP8B2, ATP8B4, or MTP can be introduced into and expressed in nematodes by techniques common in the art, such as those disclosed in Section 5.3, and used in the screening assays of the invention (Harris et al., 2003, Biochem. Biophy. Acta 1633:127-131). For example, an expressible form of a human gene encoding a MOLL homolog, e.g., ATP8B1, ATP8B2, ATP8B4, or MTP, can be expressed in a worm comprising a mutant dsc-3 or dsc-4 genotype, and/or exhibiting a mutant dsc-3 or dsc-4 phenotype to determine the effect of the human gene in restoring the wild type phenotype. Test compounds can then be added to the worms that comprise the human homolog of MOLL genes (e.g, dsc-3 or dsc-4) so that compounds that disrupt the function of the human genes can be identified.

In a related embodiment, the drug screening assays can be conducted with test nematodes in which the expression of one or more specific genes (in addition to those that generate the initial phenotype in the test nematode) are reduced. This can be accomplished by methods known in the art such as RNA-mediated interference.

In yet another embodiment, the invention also provides assays that use techniques which measure directly the outcome of the relevant metabolic states or events that are associated with the phenotypes. Typically, these assays measure the presence, concentration, and distribution of certain metabolites, such as lipids and lipoproteins, in a part of or the whole nematode.

The target screening and drug screening assays of the invention as outlined above share common basic elements. First, the assays employ test nematodes of a known genotype that display one or more characterized phenotypes, wherein the phenotypes indicate the presence of certain metabolic states or events. Second, the assay requires a means for detecting the presence of a phenotype or a change in the phenotype, or a means for measuring a change in the phenotype, many of which are quantitative in nature. It is envisaged that these common elements can be combined to produce a variety of assays of the invention. This is one of the advantages of the drug discovery platform of the invention which is flexible, productive, and open-ended. For example, a nematode of a particular phenotype can be used in target screening assays as well as drug screening assays, the difference being the treatment of the nematode received, i.e., either mutagenesis, specific mRNA reduction, or contact with a test compound. The changes in the phenotypes of the treated nematodes can be assessed by the same set of techniques. Accordingly, a particular means or method developed for assessing a specific phenotype of a test nematode can be used in many different assays.

In various nematode assays of the invention, specific phenotypes are detected, observed, and/or measured, and compared with other experimental nematodes and control nematodes. The quantitative nature of many of the phenotypes allows correlation with lipid/lipoprotein level in the nematode. The following sections describe the nematode assays of the invention which are organized by the phenotype of interest.

5.4.1 Assay Technology

Assays of the invention can be used with many species of microscopic nematode worms—Nematoda, Rhabditidae, preferably Caenorhabditis species, such as C. elegans, and C. briggsae. Most preferably, C. elegans is used. There are many advantages in using C. elegans as a model organism. First, C. elegans has a short life-cycle of about 3 days which allows these nematodes (including mutants, transgenics and/or stable lines thereof) to be grown and developed quickly and in high numbers. Because of this short life span, in C. elegans based-assays, compounds may be tested over one or more, and up to essentially all, stages of development, without any problems associated with compound stability. Second., C. elegans is transparent, thus allowing for visual or non-visual inspection of internal organs and internal processes, and also the use of markers such as fluorescent reporter proteins, even while the nematodes are still alive. Accordingly, such inspection may be carried out in automated fashion using suitable equipment such as plate readers. Finally, the genetics of C. elegans is well known and the genome is sequenced, thus lending the nematode to many techniques and tools for genetic analysis, such as DNA and protein microarrays.

Techniques for transforming, handling, growing, maintaining and storing (e.g., as frozen samples) C. elegans are well established in the art, and are described for example, in W. B. Wood et al., “The nematode Caenorhabditis elegans”, Cold Spring Harbor Laboratory Press (1988) and Riddle et al., “C. elegans II”, Cold Spring Harbor Laboratory Press (1997); C. elegans: A Practical Approach by I. A. Hope, Oxford University Press, England 1999; each of which is incorporated herein by reference in its entirety.

General techniques and methodology for performing in vivo assays using the nematode worm Caenorhabditis elegans (C. elegans) as a model organism have been described in the art, such as but not limited to Rand and Johnson, Chapter 8, Vol. 84 “Caenorhabditis elegans: Modern Biological Analysis of An Organism”, Ed. Epstein and Shakes, Academic Press, 1995, WO 98/51351, W099/37770, WO 00/34438, WO/00/01846, WO 00/63427, WO 00/63425, WO 00/63426, WO 01/88532, and WO 01/94627, each of which is incorporated herein by reference in its entirety. As described in these applications, one of the main advantages of assays involving the use of C. elegans is that such assays can be carried out in multi-well plate format (with each well usually containing a sample of between 1 and 100 nematodes).

Generally, in the assays described below, the nematodes are incubated in suitable vessel or compartment, such as a well of a multi-well plate, on a suitable medium which may be a solid, semi-solid, viscous or liquid medium, with liquid and viscous media usually being preferred for assays in multi-well plate format. The medium is also seeded with bacteria which serves as food for the nematodes. In assays, where the nematodes are contacted with one or more test compounds, the compound can be added to the medium on or in which the nematodes grow, or the nematodes can be soaked in a solution containing the compound for various time intervals. After a suitable incubation time (i.e., sufficient for the compound to have its effect, if any, on the nematodes), the nematodes are then subjected to detection or measurement by visual inspection or by one or more techniques appropriate to the phenotype of interest. Since the nematodes can move around in the medium, the nematodes may optionally be killed or paralyzed prior to the analysis; this additional step may be used to keep the distribution of nematodes in the compartment or well uniform for signal detection. A change in the phenotype as compared to a control not contacted with the test compound is an indication of the influence of the compound on the nematode. Many of the manipulations can be automated such as by using suitable robotics and high throughput assay technologies known in the art. Preferably, in automated assays, techniques involving a non-visual detection method, such as measurement of fluorescence or nucleic acid hybridization can be used.

In many screening assays of the invention, non-visual methods of detection and measurement are used to determine the effect of a gene mutation, a RNA interference molecule, or a test compound on a phenotype. In various embodiments, these methods are based on the association of the phenotype of interest with the expression or a change in expression of one or more indicator genes (e.g., genes associated with a particular tissue type, developmental stage or behavior, etc.).

In one embodiment, the methods of the invention monitors the expression levels of the indicator genes and uses the gene expression profile of one or more indicator genes to determine the manifestation of a phenotype as well as quantitative aspects of the phenotype. A list of exemplary indicator genes is provided herein below for each of the phenotypes used in the assays of the invention.

In another embodiment, the methods of the invention exploits the activity of the regulatory sequences, such as promoters and enhancers, of an indicator gene for the generation of a signal in parallel to the manifestation of the phenotype. The detectable signal is produced by a reporter molecule, either by itself or by the use of accessory molecules. As used herein, a reporter is the gene product of a reporter gene which is operably associated with the regulatory sequence(s) of an indicator gene. The reporter can be a non-nematode protein or a fusion protein, for example, of a non-nematode protein with a part of or an entire indicator gene product. “Operably-associated” or “operably-linked” refers to an association in which the promoter and the reporter gene sequence(s) are joined and positioned in such a way as to permit transcription in nematode. Examples of reporter molecules are listed in Section 5.4.1.2.

Expression of one or more indicator genes can be assessed directly by detecting and/or measuring the levels of messenger RNA of the respective indicator genes, or the levels of the indicator gene products. Where a reporter gene is operably associated with an indicator gene regulatory region in a test nematode, the transcriptional and/or translational activity of the region can be determined by measuring the level of the reporter gene mRNA, the level of reporter, or the signal generated by the reporter.

Alternatively, expression of an indicator gene can be determined indirectly by detecting and measuring the production or processing of other metabolite(s), where the production or processing reflects the expression and functional activity of the indicator gene product. This approach is particularly applicable if the indicator gene product is an enzyme. Such metabolites can be products of biochemical reactions that are downstream of a pathway in which the indicator gene product is involved. Such alternative methods of detecting the expression of the indicator gene or functional nucleotide sequence are intended to fall within the scope of the invention.

Expression of an indicator gene or reporter gene in test nematodes can be detected or measured by nucleic acid-based detection techniques. Ribonucleic acid (RNA) from the test nematodes can be used as the starting point for such assay techniques, and can be isolated according to standard nucleic acid preparation procedures which are well known to those of skill in the art. RNA can be used in hybridization or amplification assays. If a sufficient quantity of the test nematodes can be obtained, standard Northern analysis can be performed to determine the level of messenger RNA expression of the indicator gene or reporter gene.

Hybridization assays, such as Northern blot analysis, dot blot or slot blot hybridization, can involve for example, contacting and incubating RNA derived from test nematodes with one or more labeled nucleic acid probes under conditions favorable for the specific annealing of these probes to their complementary sequences within the indicator gene. Preferably, the lengths of nucleic acid probes are at least 15 nucleotides. After incubation, all non-annealed nucleic acids are removed from the probe:indicator transcript hybrid. The presence of nucleic acids which have hybridized, if any such molecules exist, is then detected. Appropriate nucleic acid probes to various indicator genes can be obtained from public and commercial sources, or synthesized by well known chemical methods, or by amplification and subcloning into plasmid vectors. Nucleotide sequences of indicator genes can be obtained from databases such as the Wormbase as described in Harris et al., 2004, Nucleic Acids Research, 32, Database issue D41 1-D417.

Using such a detection scheme, the nucleic acid of the indicator genes or reporter gene can be immobilized, for example, to a solid support such as a membrane, a glass surface, a silicon substrate, or a plastic surface such as that on a microtiter plate, glass slide, silicon wafer, or polystyrene beads. In this case, after incubation, non-annealed, labeled nucleic acid probes can be removed by washing the solid support. Detection or measurement of the remaining, annealed, labeled indicator nucleic acid reagents is accomplished using standard techniques well-known to those in the art, such as autoradiography, phosphorimaging, fluorescence measurement, light scattering, etc., depending on the labels used. Any appropriate isotopic and nonisotopic labels can be used. The amount of indicator gene transcript to which the nucleic acid probes have annealed can be compared to the amount obtained from control nematodes which have not been mutated or exposed to RNAi molecules or test compounds.

Alternative to Northern blot analysis, RNA obtained from test nematodes containing a complex mixture of transcripts, or cDNA prepared from such RNA, can be used as probes in hybridization assays of the invention. The RNA or cDNA probes, labeled isotopically or nonisotopically by standard techniques, are allowed to contact and incubate with a selected panel of indicator genes associated with a phenotype. Nucleic acids comprising the sequence of these genes or a subsequence thereof can be immobilized onto a solid support in the form of an array and presented to the labeled probes. Incubation conditions favoring the specific annealing of the labeled probes to their respective complementary sequences within the genes in the panel are employed. If the transcripts of an indicator gene which is included in the panel is present in the test nematode, a proportion of the transcripts which is labeled will hybridize to the immobilized nucleic acids of the indicator gene. After incubation, all non-annealed probes are removed, and the presence and amount of labeled probe which have hybridized to the genes in the panel is then detected. Detection or measurement of the remaining, annealed, labeled nucleic acid probe for each of the genes is accomplished using standard techniques well-known to those in the art, such as autoradiography, phosphorimaging, fluorescence measurement, etc. See, for example, Lockhart et al., 1996, Nature Biotechnol 14:1675-1680; and Ferguson et al., 1996, Nature Biotechnol, 14:1681-1684.

To facilitate parallel and high throughput analysis of hybridization results, the nucleic acids of the indicator genes can be immobilized at a high density on a solid support in a spatially distinguishable format to form an array. See, for example, the approaches described in Reinke, 2002, Nat Genet 32 Suppl:541-6 and Hill et al., 2000, Science 290:809-12, each of which is incorporated herein by reference in its entirety. Any method for producing arrays or microarrays of nucleic acids can be used. For example, DNA may be spotted directly onto a solid support, such as a porous membrane or glass (Zhao et al., 1995, Gene 156:207-213; Shalon et al., 1996, Genome Research 6:639-645), or transferred to a surface by inkjet technology (Blanchard et al., 1996, Biosens. Bioelectron. 11:687-690). Alternatively, oligonucleotides that comprise a subsequence of the indicator gene can be synthesized directly on derivatized glass or silicon using a combination of photolithography and oligonucleotide chemistry (Fodor et al., 1991, Science 251:767-773; Fodor et al., 1993, Science 364:555-556). DNA arrays useful in this parallel, high throughput approach are available commercially (e.g., from Affymetrix, Inc. Santa Clara, Calif.).

One of the advantages of this approach is that the expression of many indicator genes in a sample of test nematodes can be assessed in one simple hybridization assay. Accordingly, the methods of the invention can be used to assess the manifestation of a phenotype based on the expression of a large number of indicator genes simultaneously.

Alternative detection methods for the detection of indicator or reporter gene specific transcript, can involve their amplification, e.g., by polymerase chain reaction (PCR; U.S. Pat. No. 4,683,202), followed by the detection of the amplified molecules using techniques well known to those of skill in the art. The resulting amplified sequences can be compared to those which would be obtained from test nematodes not exposed to test compound or to RNAi molecule, or not mutated. Quantitative PCR techniques can also be used to determine the absolute amount of indicator/reporter gene transcript or the concentration of transcript relative to a standard (Wang et al., 1989, PNAS 86:9717-9721; Gilliland et al., 1990, PNAS 87:2725-2729). Multiplex PCR can be performed in which more than one indicator gene transcript, or more than one portion of an indicator gene transcript can be amplified from one sample simultaneously.

In one embodiment of such a detection scheme, cDNAs are synthesized from the RNAs of indicator/reporter genes (e.g., by reverse transcription of the RNA molecule into cDNA). A sequence within the cDNA is then used as the template for a nucleic acid amplification reaction, such as a PCR amplification reaction, or the like. The preferred lengths of PCR primers are at least 9-30 nucleotides. For detection of the amplified product, the nucleic acid amplification can be performed using radioactively or non-radioactively labeled nucleotides. Alternatively, enough amplified product can be made such that the product can be visualized by standard ethidium bromide staining or by utilizing any other suitable nucleic acid staining method.

Techniques that involve amplification and hybridization are particularly useful when not all the indicator gene transcripts are fully characterized. For example, techniques such as but not limited to differential display by PCR (Liang et al., 1992, Science 257:967-971; Pardee et al., U.S. Pat. No. 5,262,311), and serial analysis of gene transcript (SAGE; Velculescu et al., 1995, Science 270:484-487) may be used.

Those skilled in the art will be able to determine operative and optimal conditions for the above-described techniques by employing routine experimentation.

The present invention also provides protein-based screening assays which are based on the physical, immunological or functional properties of the indicator gene product, reporter molecule, fusion protein or metabolite. The indicator gene product, reporter molecule or metabolite can be isolated and purified by standard methods including chromatography and high pressure liquid chromatography based on, for example, ion exchange, affinity binding, size exclusion and hydrophobic interactions. Other standard techniques such as centrifugation, differential solubility and one- and two-dimensional gel electrophoresis can also be used. The protein isolation methods employed herein may, for example, be such as those described in Harlow and Lane (Harlow and Lane, 1988, “Antibodies: A Laboratory Manual”, Cold Spring Harbor Laboratory Press, Cold Spring Harbor, N.Y), which is incorporated herein by reference in its entirety.

Antibodies, or fragments of antibodies can be used to quantitatively or qualitatively detect by immunospecific binding the presence of indicator gene product, reporter molecule, fusion protein or metabolite, including lipoproteins and oxidized lipoproteins. This can be accomplished, for example, by immunofluorescence techniques employing a fluorescently labeled antibody coupled with light microscopic, flow cytometric, or fluorimetric detection.

In various embodiments, lipophilic dyes and stains can also be used to quantitatively or qualitatively detect lipids, lipoproteins, and metabolites that are produced by the test nematodes. The dyes and stains allow changes in the concentrations and distribution of lipids and lipoproteins in the medium, in the tissues of the nematodes, eggs, larvae, and other life stages to be detected and/or measured. The signal generated by such dyes and stains are colorimetric, and preferably, fluorescent, that can be observed by microscopy or measured by various means such as a fluorescence microplate reader. In various embodiments, one or more lipophilic compounds are used to label the assay reactions and/or the nematodes, and a specific pattern or profile of labeling is detected. Preferably, when fluorescent lipophilic dyes are used, a specific fluorescent profile or signature, such as a particular ratio of wavelength emissions under specific conditions of excitation, can be used. For example, fluorescent cholesterol analog such as dehydroergosterol, and cholesterol-binding polyene antibiotic filipin have been used to study cholesterol distribution and trafficking in living cells (Mukherjee et al., 1998, Biophysical J., 75:1915-1925). Dyes can also be incorporated into lipoprotein and used as a tracer, such as 3,3′-dioctadecylindocarbocyanine iodide (dil[3]) mixed with low density lipoprotein (LDL) to form the highly fluorescent LDL derivative dil(3)-LDL (Barak and Webb, 1981, J Cell Biol 903:595-604). Cholesteryl esters consist of a fatty acid esterified to the 3-hydroxyl group of cholesterol. These nonpolar species are the predominant lipid components of atherosclerotic plaque and low- and high-density lipoprotein (LDL and HDL) cores. Commercial suppliers of fluorescent dyes, such as Molecular Probes, Inc. provide cholesteryl esters of fluorescent labeled fatty acids—BODIPY FL C12 (C-3927), BODIPY 542/563 C11 (C-12680), BODIPY 576/589 C11 (C-12681) and of diphenylhexatrienylpropionic acid which can be adopted for use in tracing as well as measuring the levels of lipids and lipoproteins in the assay reactions and/or in nematodes.

Flow cytometry and fluorescence activated cell sorting (FACS) are well-known method for assaying and separating particles based on their fluorescent properties and dimensions (Kamarch, 1987, Methods Enzymol, 151:150-165). High throughput systems based on these techniques are available (e.g., COPAS BIOSORT by Union Biometrica, Somerville, Mass. US) for analyzing and sorting live/dead nematodes, nematode eggs, dauer larvae, and different life stages of nematodes are available. The technique may use time of flight which is an indicator of length, and/or extinction which is an indicator of size and internal structure as sort parameters. Moreover, mixed populations of test nematodes can be sorted by labeling with multiple fluorescent labels that emit at different wavelengths. This technique allows the separation of test nematodes from the growth medium which may contain bacteria, feces, and other unused metabolites. Sorted nematode can be directly deposited into individual wells of multi-well plates for further analysis. Flow cytometry and FACS can be particularly useful in assays that require determination of defecation rate, egg-laying rate, etc.

5.4.1.1 Indicator Genes

The invention provides for indicator genes to indirectly monitor the developmental stage or behavior of a C. elegans. Through the use of indicator genes, direct visual inspection of a C. elegans to determine developmental stage or performance of a behavior can be circumvented in favor of methods more amenable to high throughput technology. Aspects of the C. elegans phenotypes monitored in the assays of the invention can be translated into indicator gene expression.

As used herein the term “indicator genes” refers to those nematode genes that vary in level, location or timing of expression in the nematode at different times during development, under different circumstances, or during a particular behavior. The timing and location of expression as well as the relative expression levels of the gene products can be interpreted to indicate various information about the nematode (e.g., if a particular tissue type has developed, if a particular developmental stage has been achieved, if a particular behavior is being performed, etc.). Indicator genes exclude the target gene(s) and genes that are manipulated or mutated in the test nematode as a part of the assay. Although it is preferred, it is not required that an indicator gene is associated exclusively with only one tissue type, time in development or aspect of a behavior. For example, the indicator gene expression may accumulate over time but is greatly enriched at a particular time in development. The invention provides that, in certain embodiments, even if the expression of each individual indicator genes is not specific, the pattern or profile of expression of a number of indicator genes can be used to determine a specific piece of information about a nematode.

In one embodiment, an assay of the invention is based on rate of germline development. In a specific embodiment, germline development is monitored by monitoring the expression of one or more indicator genes specifically expressed in gametes.

In another embodiment, an assay of the invention is based on rate of post-embryonic development. In a specific embodiment, post-embryonic development is monitored by monitoring when an indicator gene associated with a particular stage in post embryonic development is expressed. In another specific embodiment, post-embryonic development is monitored by monitoring the relative size of various tissues in comparison to each other. For example, during post-embryonic development, the size of the pharynx in relation to the rest of the body decreases while the relative size of the germline and somatic gonads increases. By monitoring two or more tissue types (e.g., by monitoring indicator genes expressed exclusively or predominantly in each tissue type) whose relative size changes during development, rate of development can be followed.

In another embodiment, an assay of the invention is based on rate of embryonic development. In a specific embodiment, embryonic development is monitored by monitoring when an indicator gene associated with a particular stage in embryonic development is expressed.

Examples of somatic indicator genes including, but not limited to, myo-3 (body wall muscle), elt-2 (gut), myo-2 (pharynx), dpy-7 (hypodermis). Examples of tissue and cell specific indicator genes have been described in publicly accessible databases (e.g. Wormbase, http://www.wormbase.org/; NEXTDB, http://nematode.lab.nig.ac.jp/; The Hope Laboratory Expression Pattern Database, http://129.11.204.86:591/default.htm).

5.4.1.2 Reporter Genes

The invention provides for reporter genes to monitor the expression of molecules (e.g., nucleic acids or polypeptides) of interest. In one embodiment, the molecules of interest are indicator genes (as described above). In another embodiment, the molecules of interest are MOLLs. In a specific embodiment, expression of the MOLL nucleic acids and polypeptides can be monitored to ascertain effects of test compounds in order to aid in the identification of agents of the invention.

In general, changes in the amount or localization of a reporter gene product is indicative of the changes that the molecule of interest is exhibiting due to e.g., progression through development or incubation with a candidate compound. In one embodiment, a transgenic animal is made which expresses one or more reporter genes under the control of a promoter or enhancer of a molecule of interest. Such animals may be use in screening assays of the invention.

Reporter genes include, but are not limited to, luciferase, green fluorescent protein, beta-galactosidase, chloramphenicol acetyltransferase, and alkaline phosphatase. Such methods are well known to one of skill in the art. In a preferred embodiment, the reporter gene is easily assayed and has an activity which is not normally found in the host cell.

In one embodiment, luciferase is the reporter gene. Luciferases are enzymes that emit light in the presence of oxygen and a substrate (luciferin) and which have been used for real-time, low-light imaging of gene expression in cell cultures, individual cells, whole organisms, and transgenic organisms (reviewed by Greer & Szalay, 2002, Luminescence 17:43-74).

As used herein, the term “luciferase” is intended to embrace all luciferases, or recombinant enzymes derived from luciferases which have luciferase activity. The luciferase genes from fireflies have been well characterized, for example, from the Photinus and Luciola species (see, e.g., International Patent Publication No. WO 95/25798 for Photinus pyralis, European Patent Application No. EP 0 524 448 for Luciola cruciata and Luciola lateralis, and Devine et al., 1993, Biochim. Biophys. Acta 1173:121-132 for Luciola mingrelica). Other eucaryotic luciferase genes include, but are not limited to, the sea panzy (Renilla reniformis, see, e.g., Lorenz et al., 1991, PNAS 88:4438-4442), and the glow worm (Lampyris noctiluca, see e.g., Sula-Newby et al., 1996, Biochem J. 313:761-767). Bacterial luciferin-luciferase systems include, but are not limited to, the bacterial lux genes of terrestrial Photorhabdus luminescens (see, e.g., Manukhov et al., 2000, Genetika 36:322-30) and marine bacteria Vibrio fischeri and Vibrio harveyi (see, e.g., Miyamoto et al., 1988, J. Biol. Chem. 263:13393-9, and Cohn et al., 1983, PNAS 80:120-3, respectively). The luciferases encompassed by the present invention also includes the mutant luciferases described in U.S. Pat. No. 6,265,177.

In another embodiment, green fluorescent protein (“GFP”) is the reporter gene. GFP is a 238 amino acid protein with amino acids65 to 67 involved in the formation of the chromophore which does not require additional substrates or cofactors to fluoresce (see, e.g., Prasher et al., 1992, Gene 111:229-233; Yang et al., 1996, Nature Biotechnol. 14:1252-1256; and Cody et al., 1993, Biochemistry 32:1212-1218).

As used herein, the term “green fluorescent protein” or “GFP” is intended to embrace all GFPs (including the various forms of GFPs which exhibit colors other than green), or recombinant enzymes derived from GFPs which have GFP activity. The native gene for GFP was cloned from the bioluminescent jellyfish Aequorea victoria (see, e.g., Morin et al., 1972, J. Cell Physiol. 77:313-318). Wild type GFP has a major excitation peak at 395 nm and a minor excitation peak at 470 nm. The absorption peak at 470 nm allows the monitoring of GFP levels using standard fluorescein isothiocyanate (FITC) filter sets. Mutants of the GFP gene have been found useful to enhance expression and to modify excitation and fluorescence. For example, mutant GFPs with alanine, glycine, isoleucine, or threonine substituted for serine at position 65 result in mutant GFPs with shifts in excitation maxima and greater fluorescence than wild type protein when excited at 488 nm (see, e.g., Heim et al., 1995, Nature 373:663-664; U.S. Pat. No. 5,625,048; Delagrave et al., 1995, Biotechnology 13:151-154; Cormack et al., 1996, Gene 173:33-38; and Cramer et al., 1996, Nature Biotechnol. 14:315-319). The ability to excite GFP at 488 nm permits the use of GFP with standard fluorescence activated cell sorting (“FACS”) equipment. In another embodiment, GFPs are isolated from organisms other than the jellyfish, such as, but not limited to, the sea pansy, Renilla reriformis.

Techniques for labeling cells with GFP in general are described in U.S. Pat. Nos. 5,491,084 and 5,804,387; Chalfie et al., 1994, Science 263:802-805; Heim et al., 1994, PNAS 91:12501-12504; Morise et al., 1974, Biochemistry 13:2656-2662; Ward et al., 1980, Photochem Photobiol. 31:611-615; Rizzuto et al., 1995, Curr. Biology 5:635-642; and Kaether & Gerdes, 1995, FEBS Lett. 369:267-271. The expression of GFPs in E. coli and C. elegans is described in U.S. Pat. No. 6,251,384. The expression of GFP in plant cells is discussed in Hu & Cheng, 1995, FEBS Lett. 369:331-33, and GFP expression in Drosophila is described in Davis et al., 1995, Dev. Biology 170:726-729.

In another embodiment, beta galactosidase (“β-gal”) is used as a reporter gene. βgal is an enzyme that catalyzes the hydrolysis of β-galactosides (e.g., lactose) as well as galactoside analogs (e.g., o-nitrophenyl-β-D-galactopyranoside (“ONPG”) and chlorophenol red-β-D-galactopyranoside (“CPRG”)) (see, e.g., Nielsen et al., 1983 PNAS 80:5198-5202; Eustice et al., 1991, Biotechniques 11:739-742; and Henderson et al., 1986, Clin. Chem. 32:1637-1641).

As used herein, the term “beta galactosidase” or “β-gal” is intended to embrace all β-gals, including lacZ gene products, or recombinant enzymes derived from β-gals which have β-gal activity. The β-gal gene functions well as a reporter gene because the protein product is extremely stable, resistant to proteolytic degradation in cellular lysates, and easily assayed. In an embodiment where ONPG is the substrate, β-gal activity can be quantitated with a spectrophotometer or microplate reader to determine the amount of ONPG converted at 420 nm. In an embodiment when CPRG is the substrate, β-gal activity can be quantitated with a spectrophotometer or microplate reader to determine the amount of CPRG converted at 570 to 595 nm. In yet another embodiment, the β-gal activity can be visually ascertained by plating bacterial cells transformed with a β-gal construct onto plates containing Xgal and IPTG. Bacterial colonies that are dark blue indicate the presence of high β-gal activity and colonies that are varying shades of blue indicate varying levels of β-gal activity.

In one embodiment, chloramphenicol acetyltransferase (“CAT”) is used as a reporter gene. CAT is commonly used as a reporter gene in mammalian cell systems because mammalian cells do not have detectable levels of CAT activity. The assay for CAT involves incubating cellular extracts with radiolabeled chloramphenicol and appropriate co-factors, separating the starting materials from the product by, for example, thin layer chromatography (“TLC”), followed by scintillation counting (see, e.g., U.S. Pat. No. 5,726,041).

As used herein, the term “chloramphenicol acetyltransferase” or “CAT” is intended to embrace all CATs, or recombinant enzymes derived from CAT which have CAT activity. While it is preferable that a reporter system which does not require cell processing, radioisotopes, and chromatographic separations would be more amenable to high through-put screening, CAT as a reporter gene may be preferable in situations when stability of the reporter gene is important. For example, the CAT reporter protein has an in vivo half life of about 50 hours, which is advantageous when an accumulative versus a dynamic change type of result is desired.

In another embodiment, secreted alkaline phosphatase (“SEAP”) is used as a reporter gene. SEAP enzyme is a truncated form of alkaline phosphatase, in which the cleavage of the transmembrane domain of the protein allows it to be secreted from the cells into the surrounding media. In a preferred embodiment, the alkaline phosphatase is isolated from human placenta.

As used herein, the term “secreted alkaline phosphatase” or “SEAP” is intended to embrace all SEAP or recombinant enzymes derived from SEAP which have alkaline phosphatase activity. SEAP activity can be detected by a variety of methods including, but not limited to, measurement of catalysis of a fluorescent substrate, immunoprecipitation, HPLC, and radiometric detection. The luminescent method is preferred due to its increased sensitivity over calorimetric detection methods. The advantages of using SEAP is that a cell lysis step is not required since the SEAP protein is secreted out of the cell, which facilitates the automation of sampling and assay procedures. A cell-based assay using SEAP for use in cell-based assessment of inhibitors of the Hepatitis C virus protease is described in U.S. Pat. No. 6,280,940.

5.4.2 C. elegans-Based Assays

5.4.2.1 Assays Based on Defecation Rate

The invention features assays that use the length of defecation cycle as a test phenotype. In C. elegans, defecation is effected by a stereotyped Defecation Motor Program (DMP). The DMP consists of three distinct steps: the posterior body muscle contraction (pBoc), the anterior body muscle contraction (aBoc), and the expulsion (Exp), which consists of the enteric muscle contractions (EMC) (Thomas et al, 1990, Genetics 124: 855-872.). The defecation cycle length is defined as the duration between the pBoc steps of two consecutive defecations. In wild-type animals (in presence of adequate food), the defecation cycle length is ˜56 seconds, with a standard deviation of ±3.4 sec (at 20° C.). A mutation that affects the length and/or periodicity of the defecation cycle (e.g., clk-1 mutation) can be used in this assay.

In one embodiment, mutant clk-1 is used in the assay. As previously described (Felkai et al., 1999, EMBO J 18: 1783-1792 and Wong et al., 1995, Genetics 139: 1247-1259), in clk-1 mutants the defecation cycle is both increased in length and more irregular: in clk-1(qm30) animals, the cycle length is 88 sec, with a standard deviation of ±14 sec, and in the weaker allele clk-1(e2519), it is 77 sec, with a standard deviation of ±7 sec (at 20° C.).

In addition to defecation cycle length at 20° C., the ability of the mutant C. elegans to re-adjust the length of their defecation cycle after being shifted to a new temperature is affected. In contrast to wild type worms, clk-1 mutants are unable to re-adjust the length of their defecation cycle after being shifted to a new temperature. When clk-1 mutants are transferred from either 20° C. to 25° C. or from 20° C. to 15° C. there is no change in the mean cycle length. Rescue of this aspect of the mutant phenotype would restore the increase in cycle length at lower temperatures (e.g., 15° C.) and the decrease in cycle length at higher temperatures (e.g., 25° C.). For example, test compounds added to clk-1 mutant worm assays can be identified based on their ability to phenocopy a clk-1/dsc-3 double mutant worm or a clk-1/dsc-4 double mutant. In certain embodiments, the phenotypes of the mutant worms and worms with the test compound are measured under higher and lower temperatures.

In yet other related embodiments, one or more genes or mutants thereof that are characterized by an alteration in nematode defecation cycle can be used in the screening assays of the invention.

In another embodiment, the method comprises detecting the expression of a reporter encoded by a reporter gene that is operably linked to the regulatory sequences of an indicator gene of which the expression level is associated with defecation. Additionally, an expression profile of indicator genes may be used. Exemplary indicator genes of which the promoter can be used include those described in publicly accessible databases (e.g. Wormbase, http://www.wormbase.org/; NEXTDB, http://nematode.lab.nig.ac.jp/; The Hope Laboratory Expression Pattern Database, http://129.11.204.86:591/default.htm).

5.4.2.2 Assays Based on Rate of Germline Development

The invention features assays that use the rate of germline development as a test phenotype. In wild type nematodes, the soma and germline develop at the same rate. A mutation that affects the synchronicity of soma/germline development (e.g., clk-1 mutation) can be used in this assay. In one embodiment, mutant clk-1 is used in the assay. clk-1 mutant worms display a heterochronic phenotype which is characterized by a disruption in the synchronization between the rate of development of the germline and the soma at large such that the germline develops more slowly than the soma. As demonstrated by the experiments described in Section 7.2, the suppression of this phenotype was associated with a lower level of cholesterol intake, a reduced level of production and/or secretion of LDL-like protein, or a lower level of oxidation of the LDL-like protein. An accelerated rate or a restoration to normal rate of germline development as compared to control nematodes is scored as a positive in this assay. The rate of germline development can be measured in a number of ways, including but not limited to, time at which the peak egg laying rate is attained, the time at which there are oocytes at the proximal end of the posterior germline, and the time at which there is an onset of gametogenesis.

In one embodiment, peak egg laying rate is used to screen for suppressors of a clk-1 mutation. clk-1 mutant C. elegans reach their peak of egg laying rate three times more slowly than wild type C. elegans (72 hours versus 24 hours after molting into adults). C. elegans clk-1 mutants that have been mutagenized can be examined for those that reach peak egg laying rate in about 24 hours after molting into adults.

In another embodiment, oocytes at the proximal end of the posterior germline are used to screen for suppressors of a clk-1 mutation. The C. elegans adult hermaphrodite gonad consists of two U-shaped arms (an anterior and a posterior), each of which terminates in a spermatheca. The two spermathecae (the distal end) join the gonad arms to the uterus, which stores the fertilized eggs, and fuse at the vulva (the proximal end). The stage of development of the germline is polarized along the distal-proximal axis. Most of spermatogenesis takes place in the proximal gonad. For oogenesis, the distal arm of each gonad forms a syncytium that contains the germ cell nuclei undergoing mitosis. Moving proximally, germ cells exit the mitotic cycle and enter into, and progress through the first stages of meiosis. At 6 hours after the adult molt, wild-type C. elegans have oocytes at the proximal end of the anterior and posterior germline. In comparison, the onset of oogenesis is dramatically delayed in clk-1 mutants. Only about 3% of clk-1 C. elegans had initiated oogenesis by 6 hours after adult molt. clk-1 C. elegans that have been mutagenized can be examined for those that initiated oogenesis by 6 hours after adult molt.

In yet another embodiment, the onset of gametogenesis is used to screen for suppressors of clk-1. Wild-type C. elegans complete primary spermatocyte formation 1.5 hours after the adult molt. In contrast, clk-1 mutant C. elegans have not started or are just beginning primary spermatocyte formation 1.5 hours after the adult molt. clk-1 C. elegans that have been mutagenized can be examined for those that complete primary spermatocyte formation 1.5 hours after the adult molt.

In yet another embodiment, the method comprises detecting the expression of a reporter encoded by a reporter gene that is operably linked to the regulatory sequences of an indicator gene of which the expression level is associated with germline development. Additionally, an expression profile of indicator genes may be used. Exemplary indicator genes of which the promoter can be used include, but are not limited to, ark- 1, itr- 1, and let 60. The experiments described in Section 5.4.2 demonstrate use of such indicator genes the expression level of which is associated with germline development. Other examples of exemplary indicator genes of which the promoter can be used include those disclosed in Reinke et al., 2000, Mol. Cell 6:605-16; Colaiacovo et al., Genetics 2002 September; 162:113-28 or in publicly available databases (e.g. Wormbase, http://www.wormbase.org/; NEXTDB, http:H/nematode.lab.nig.ac.jp/; The Hope Laboratory Expression Pattern Database, http://129.11.204.86:591/default.htm).

5.4.2.3 Assays Based on Rate of Embryonic Development

The invention features assays that use the rate of embryonic development as a test phenotype. A synchronous population of C. elegans can be obtained by dissecting 2-4 cell stage embryos from mothers and placing them onto a new plate (see e.g., Wong et al., 1995, Genetics 139: 1247-1259). In wild type nematodes, embryonic development takes 13 hours. A mutation that affects the rate of embryonic development (e.g., clk-1) can be used in the assay.

In one embodiment, mutant clk-1 is used in the assay. Embryonic development of clk-1 mutant C. elegans takes between 17 and 22 hours.

In another embodiment, the method comprises detecting the expression of a reporter encoded by a reporter gene that is operably linked to the regulatory sequences of an indicator gene of which the expression level is associated with embryonic development. Additionally, an expression profile of indicator genes may be used. Exemplary indicator genes of which the promoter can be used include those described in publicly accessible databases (e.g. Wormbase, http://www.wormbase.org/; NEXTDB, http://nematode.lab.nig.ac.jp/; The Hope Laboratory Expression Pattern Database, http://129.11.204.86:591/default.htm).

5.4.2.4 Assays Based on Rate of Post-Embryonic Development

The invention features assays that use the rate of post-embryonic development as a test phenotype. A synchronous population of C. elegans can be obtained by picking eggs to a new plate and examining one hour later (see e.g., Wong et al., 1995, Genetics 139: 1247-1259). Animals that hatch during this period are used for the assay. Wild type nematodes reach adulthood between 45 and 51 hours after hatching. A mutation that affects the rate of post-embryonic development (e.g., clk-1) can be used in the assay.

In one embodiment, C. elegans clk-1 mutant is used in the assay. These mutants reach adulthood between 63 and 81 hours after hatching.

In another embodiment, the method comprises detecting the expression of a reporter encoded by a reporter gene that is operably linked to the regulatory sequences of an indicator gene of which the expression level is associated with post-embryonic development. Additionally, an expression profile of indicator genes may be used. Exemplary indicator genes of which the promoter can be used include those described in publicly accessible databases (e.g. Wormbase, http://www.wormbase.org/; NEXTDB, http://nematode.lab.nig.ac.jp/; The Hope Laboratory Expression Pattern Database, http://129.11.204.86:591/default.htm).

5.4.2.5 Gene Mutations and Assays of the Invention

In certain embodiments of the methods of the invention, the methods are carried out in a C. elegans comprising a mutation in one or more gene. Such mutants provide the desirable genetic background for screening and/or validation experiments and for creating worms with multiple mutations. In preferred embodiments, the mutant C. elegans comprises a knockout (loss-of-function) or modulation of function mutation in one or more of the clk-1, dsc-3, or dsc-4 genes. According to certain aspects of the invention, the mutation is a substitution, deletion, or insertion mutation in one or more of the domains of clk-1, dsc-3, or dsc-4.

In related embodiments, the methods of the invention are carried out in a mutant C. elegans that comprises a clk-1 mutation and one or more mutation in other genes. In related embodiments, the methods of the invention are carried out in a mutant C. elegans that comprises a clk-1 mutation and one or more mutation in dsc-3. In related embodiments, the methods of the invention are carried out in a mutant C. elegans that comprises a clk-1 mutation and one or more mutation in dsc-4. Examples of mutations useful in the methods of the invention include, but are not limited to clk-1(e2519), dsc-3(qm180), and dsc-3(qm184). Non-limiting examples of such combination mutant backgrounds include clk-1(qm30)/ dsc-3(qm184).

According to one aspect of the invention, the C. elegans comprising clk-1, dsc-3, or dsc-4 mutations can further comprise one or more RNAi suppression constructs to block expression of one or more of the following: fat-2, fat-3, elo-1, elo-2, vit-2, vit-3, vit-4, vit-5 vit-6, sod-1, sod-2, sod-3, sod-4, or let-60. Expression of other genes involved in cholesterol and/or LDL metabolism, defecation cycle, rate of germline development, or ROS levels can also be blocked using RNAi to generate C. elegans with a desirable biological background for use in the methods of the invention. In related embodiments, one or more of the genes can be suppressed using other techniques commonly used in the art such as the well-known antisense, gene “knock-out,” ribozyme and/or triple helix methods.

In related embodiments, the mutant C. elegans comprises a knockout (loss-of-function) or modulation of function mutation in a gene that results in modulation of defecation cycle. Examples of such mutations include, but are not limited to, itr-1(sa73) and isp-1(qm150).

In related embodiments, the mutant C. elegans comprises a knockout (loss-of-function) or modulation of function mutation in one or more of the vit-2, vit-3, vit-4, vit-5, or vit-6 genes. These genes encode vitellogenins, which are apoB homologues, secreted by cells of the intestines of C. elegans. Vitellogenins are also known to function in yolk lipoproteins. The results presented herein in Section 8.2 indicate that vitellogenins function in lipoprotein particles that resemble the apoB-dependent LDL particles found in vertebrates and that can be distinct from the yolk lipoprotein particles. Since apoB is involved in synthesis and secretion of LDL particles in the intestines of mammals, mutants of vit genes can also be used in the methods of the invention for identifying compounds that modulate LDL uptake or cholesterol levels.

In other related embodiments, the mutant C. elegans comprises a knockout (loss-of-function) or modulation of function mutation in one or more gene that results in a change in reactive oxygen species (ROS) levels. For example, in certain embodiments of the invention, the mutant background can comprise mutations in clk-1, sod-1, sod- 2, sod-3, or sod-4. According to certain aspects of the invention, the methods of the invention may be practiced in animal models. For example, one or more of the genes, mutant forms of genes, or constructs designed to suppress the genes described herein can be transformed into an organism such as a nematode, mouse, or rat. Such transformed animals can then be used in the methods of the invention for identifying compounds.

5.4.2.6 ATPase Assays

ATPase enzymes are a large family of enzymes that are integral membrane proteins the enzymes transport agents such as metals, ions, and phospholipids across a membrane using ATP (Harris et al, 2003, Biochim. Biophys. 1633:127-131). Mutations in ATPase, such as mutations of the human ATP8B1/FIC1 gene result in a cholestatic phenotype characteristic of Byler's disease (Trauner et al, 2002, Physiol. Rev. 83:633-671). Cholestatic diseases are conditions in which bile flow within the liver is impaired, which suggests ATPase enzymes such as, but not limited to, ATP8B1/FIC1 play a role in bile acid transport and secretion. ATPase enzymes are also involved in maintaining plasma membrane phospholipid asymmetry which facilitates lipid transport across membranes (Daleke, 2003, J. Lip. Res. 44:233-242). ATP8B1/FIC1 has been expressed in CHOK1 cells and then identified in membranes of those cells where an altered the distribution of lipids in the membrane was observed (Ujhazy et al., 2001, Hepatology 34:768-775).

dsc-3, having similarities to ATPases as described in the example sections below, can be used in assays to screen for compounds that modulate its biological function by modulating ATPase enzyme activity. DSC-3 can be obtained or made by techniques known in the art. For example, DSC-3 can be isolated from membranes of recombinant or normal cells expressing dsc-3 or in cells designed to recombinantly produce DSC-3. The DSC-3 enzyme can then be contacted with an amount of ATP and a lipid in the presence and absence of a test compound and the resulting amount of ATP in the presence and absence of a test compound indicates whether the test compound modulates the activity of the enzyme. In related embodiments, the ATP is labeled. dsc-3 can also be used in assays to screen for compounds that modulate transport of lipids across a membrane. In such assays, isolated membranes or whole cells with DSC-3 containing membranes can be contacted with lipids that have been labeled in the presence and absence of a test compound. For example, if the labeled lipid is contacted to cells with DSC-3 containing membranes in the presence and absence of a test compound and a change in the intracellular and extracellular distribution of the labeled lipid is observed in the presence and absence of a test compound, then a compound that modulates DSC-3 lipid transport is identified.

dsc-3 can also be used in assays to screen for compounds that modulate, i.e., enhance or inhibit, bile acid transport and/or secretion which would be useful in the treatment and prevention of cholestatic diseases. For example, an organism which exhibits bile acid transport and/or secretion, such as a mouse, can be transformed to express dsc-3. The effects on the level of bile acid secretion can be measured by sampling bile fluid in transformed and non-transformed mice. Such transgenic mice can also be used to screen for compounds that modulate bile acid transport and/or secretion. For example, the levels of bile acid in the liver can be measured mice expressing dsc-3 in the presence and absence of a test compound, such that if the levels of bile differ in the presence and absence of the test compound, a compound that modulates bile acid is identified.

In certain embodiments of the invention, human genes that correspond to dsc-3 or dsc-4 can be expressed in transformed mice to screen for compounds that modulate bile acid transport and/or secretion which would be useful in the treatment and prevention of cholestatic diseases. In related embodiments, the human genes corresponding to dsc-3 or dsc-4 have about 10%, 20%, 30%, 40%, 50%, 60%, 70%, 80%, 90%, or greater similarity to dsc-3 or dsc-4.

5.4.3 Non C. elegans-Based Assays

The present invention also encompasses in vivo assays that do not involve C. elegans. In one embodiment, a polypeptide or agent that binds clk-1 or a MOLL of the invention is identified using a yeast-two hybrid screen. Any method known in the art can be used to perform such a two hybrid screen. One version of the two-hybrid system has been described (Chien et al., 1991, PNAS 88:9578-9582) and is commercially available from Clontech (Palo Alto, Calif.). In one embodiment, a polypeptide that binds clk-1 or a MOLL is identified. In such an embodiment, the identified polypeptide can be tested to see if it is itself a MOLL and thus useful as a target using the directed mutagenesis techniques discussed above (see Sections 5.3.1.1-5.3.1.4). Alternatively, the identified polypeptide can be used to identify agents that bind to it. In another embodiment, an agent that binds clk-1 or a MOLL is identified. In such an embodiment, the identified agent can be tested to see if it is an agent of the invention as demonstrated by activity in a screen described previously (see Section 5.3.2).

5.4.4 In Vitro Biochemical Assays

Compounds that bind to a MOLL polypeptide of the invention (e.g., dsc-4) can be identified by any method known in the art. For example, any method that detects an altered physical property (e.g., size, mobility, etc.) of a MOLL polypeptide of the invention complexed to a test compound from an unbound polypeptide of the invention can be used in the methods of the invention, including, but not limited to, electrophoresis, size exclusion chromatography, and mass spectrometry. Other methods to detect binding between MOLL polypeptides of the invention and test compounds directly can also be used, including, but not limited to, affinity chromatography, scintillation proximity assay, nuclear magnetic resonance spectroscopy, and fluorescence resonance energy transfer.

In a first embodiment, electrophoresis is used to identify test compounds capable of binding a MOLL polypeptide of the invention. In general, a MOLL polypeptide of the invention bound to a test compound is larger than an unbound MOLL polypeptide of the invention. Electrophoretic separation based on size allows for determination of such a change in size. Any method of electrophoretic separation, including but not limited to, denaturing and non-denaturing polyacrylamide gel electrophoresis, urea gel electrophoresis, gel filtration, pulsed field gel electrophoresis, two dimensional gel electrophoresis, continuous flow electrophoresis, zone electrophoresis, agarose gel electrophoresis, and capillary electrophoresis can be used.

In a preferred embodiment, an automated electrophoretic system can be used, including, but not limited to, those systems comprising a capillary cartridge (see e.g., U.S. Pat. Nos. 5,885,430; 5,916,428; 6,027,627; and 6,063,251) or a chip (see e.g., U.S. Pat. Nos. 5,699,157; 5,842,787; 5,869,004; 5,876,675; 5,942,443; 5,948,227; 6,042,709; 6,042,710; 6,046,056; 6,048,498; 6,086,740; 6,132,685; 6,150,119; 6,150,180; 6,153,073; 6,167,910; 6,171,850; and 6,186,660).

In another preferred embodiment, the electrophoretic method of separation comprises polyacrylamide gel electrophoresis, preferably non-denaturing the polyacrylamide gel electrophoresis, so as to differentiate the mobilities of the MOLL polypeptides of the invention that are either unbound or bound to a test compound. In another embodiment, the MOLL polypeptides of the invention separated by the electrophoresis are transferred to a membrane for immunoblotting. Such techniques are well known to one of skill in the art.

In a second embodiment, size exclusion chromatography is used to identify test compounds capable of binding MOLL polypeptides of the invention. Size-exclusion chromatography separates molecules based on their size and uses gel-based media comprised of beads with specific size distributions. When applied to a column, this media settles into a tightly packed matrix and forms a complex array of pores. Separation is accomplished by the inclusion or exclusion of molecules by these pores based on molecular size. Small molecules are included into the pores and, consequently, their migration through the matrix is retarded due to the added distance they must travel before elution. Large molecules are excluded from the pores and migrate with the void volume when applied to the matrix. In the present invention, a MOLL polypeptide of the invention bound to a test compound will be larger, and thus elute faster from the size exclusion column, than an unbound MOLL polypeptide.

In a third embodiment, mass spectrometry is used to identify test compounds capable of binding polypeptides of the invention. An automated method for analyzing mass spectrometer data which can analyze complex mixtures containing many thousands of components and can correct for background noise, multiply charged peaks and atomic isotope peaks is described in U.S. Pat. No. 6,147,344. The system disclosed in U.S. Pat. No. 6,147,344 is a method for analyzing mass spectrometer data in which a control sample measurement is performed providing a background noise check. The peak height and width values at each m/z ratio as a function of time are stored in a memory. A mass spectrometer operation on a material to be analyzed is performed and the peak height and width values at each m/z ratio versus time are stored in a second memory location. The mass spectrometer operation on the material to be analyzed is repeated a fixed number of times and the stored control sample values at each m/z ratio level at each time increment are subtracted from each corresponding one from the operational runs, thus producing a difference value at each mass ratio for each of the multiple runs at each time increment. If the MS value minus the background noise does not exceed a preset value, the m/z ratio data point is not recorded, thus eliminating background noise, chemical noise and false positive peaks from the mass spectrometer data. The stored data for each of the multiple runs is then compared to a predetermined value at each m/z ratio and the resultant series of peaks, which are now determined to be above the background, is stored in the m/z points in which the peaks are of significance.

In a fourth embodiment, affinity chromatography is used to identify test compounds capable of binding MOLL polypeptides of the invention. To accomplish this, a MOLL polypeptide of the invention is labeled with an affinity tag (e.g., GST, HA, myc, streptavidin, biotin) such that the MOLL polypeptide of the invention can attach to a solid support through interaction with the affinity tag and solid support medium. The tagged MOLL polypeptide of the invention is contacted with a test compound either while free in solution or while bound to a solid support. The solid support is typically comprised of, but not limited to, cross-linked agarose beads that are coupled with a ligand for the affinity tag. Alternatively, the solid support may be a glass, silicon, metal, or carbon, plastic (polystyrene, polypropylene) surface with or without a self-assembled monolayer either with a covalently attached ligand for the affinity tag, or with inherent affinity for the tag on the MOLL polypeptide of the invention. Once the complex between the MOLL polypeptide of the invention and test compound has reached equilibrium and has been captured, one skilled in the art will appreciate that the retention of bound compounds and removal of unbound compounds is facilitated by washing the solid support with large excesses of binding reaction buffer. Furthermore, retention of high affinity compounds and removal of low affinity compounds can be accomplished by a number of means that increase the stringency of washing; these means include, but are not limited to, increasing the number and duration of washes, raising the salt concentration of the wash buffer, addition of detergent or surfactant to the wash buffer, and addition of non-specific competitor to the wash buffer. Following the removal of unbound compounds, bound compounds with high affinity for the immobilized MOLL polypeptide of the invention can be eluted and analyzed. The elution of test compounds can be accomplished by any means that break the non-covalent interactions between the polypeptide of the invention and test compound. Means for elution include, but are not limited to, changing the pH, changing the salt concentration, the application of organic solvents, and the application of molecules that compete with the bound ligand. Preferably, the means employed for elution will release the compound from the MOLL polypeptide of invention, but will not effect the interaction between the affinity tag and the solid support, thereby achieving selective elution of test compound.

In a fifth embodiment, a scintillation proximity assay (“SPA”) is used to identify test compounds capable of binding to a polypeptide of the invention. In this embodiment either the polypeptide of the invention or the test compound must labeled (e.g., with a radioisotope, etc.). The unlabeled entity is attached to a surface impregnated with a scintillant. The labeled entity is then incubated with the attached unlabeled entity under conditions that allow binding. The amount of binding between a polypeptide of the invention and test compound is quantitated with a scintillation counter (Cook, 1996, Drug Discov. Today 1:287-294; Mei et al., 1997, Bioorg. Med. Chem. 5:1173-1184; Mei et al., 1998, Biochemistry 37:14204-14212). High throughput SPA screening uses microplates with scintillant either directly incorporated into the plastic (Nakayama et al., 1998, J. Biomol. Screening 3:43-48) or coating the plastic. In a preferred embodiment, such microtiter plates are used in methods of the invention comprising (a) labeling of the MOLL polypeptide of the invention with a radioactive label; (b) contacting the labeled MOLL polypeptide with a test compound, wherein the test compound is attached to a microtiter well coated with scintillant; and (c) identifying and quantifying the amount of polypeptide of the invention bound to the test compound with SPA.

In a sixth embodiment, nuclear magnetic resonance spectroscopy (“NMR”) is used to identify test compounds capable of binding MOLL polypeptides of the invention. NMR is used to identify MOLL polypeptides of the invention that are bound by a test compound by qualitatively determining changes in chemical shift, specifically from distances measured using relaxation effects. NMR-based approaches have been used in the identification of small molecule binders of protein drug targets (Xavier et al., 2000, Trends Biotechnol. 18:349-356). A strategy for lead generation by NMR using a library of small molecules has been described (Fejzo et al., 1999, Chem. Biol. 6:755-769).

In a seventh embodiment, fluorescence resonance energy transfer (“FRET”) can be used to identify test compounds capable of binding to MOLL polypeptides of the invention. In this embodiment, both the MOLL polypeptide of the invention and the test compound are labeled with a different fluorescent molecule (i.e., flourophore). A characteristic change in fluorescence occurs when two fluorophores with overlapping emission and excitation wavelength bands are held together in close proximity, such as by a binding event. One of the fluorophores used as a label will have overlapping excitation and emission spectra with the other fluorophore used as a label such that one fluorophore (the donor) transfers its emission energy to excite the other fluorophore (the acceptor). The acceptor preferably emits light of a different wavelength upon relaxing to the ground state, or relaxes non-radioactively to quench fluorescence. FRET is very sensitive to the distance between the two fluorophores, and allows measurement of molecular distances less than 10 nm (e.g., U.S. Pat. No. 6,337,183 and Matsumoto et al., 2000, Bioorg. Med. Chem. Lett. 10:1857-1861).

### 5.5 Prophylactic/Therapeutic Methods

The invention provides methods for treating, preventing, and managing a disorder associated with undesirable/abnormal levels of lipids or lipoproteins, or ROS levels by administrating to a subject in need thereof a therapeutically or prophylactically effective amount of one or more agents of the invention. The agents of the invention can be administered alone or in combination with one or more other prophylactic/therapeutic agents useful in the treatment, prevention or management of the disorder that are not MOLL-based. The subject is preferably a mammal including, but not limited to, a non-primate (e.g., cows, pigs, horses, cats, dogs, rats, etc.) and a primate (e.g., monkey, such as a cynomolgous monkey and a human). In a preferred embodiment, the subject is a human.

The methods and compositions described herein may also be applied for the amelioration of symptoms, such as an undesirable lipid profile, associated with such disorders. Examples of disorders or conditions that can treated or prevented by the methods of the invention include but are not limited to cardiovascular disorders, heart disease, atherosclerosis, blood vessel disease, cerebrovascular disorders, and obesity. For illustration purposes only, and not to limit the scope of the disorders to be treated by the methods and compositions of the invention, cardiovascular disorders are discussed herein below as an example.

Certain aspects of cardiovascular disorders are brought about, at least in part, by an excessive level of gene product, or by the presence of a gene product exhibiting an abnormal or excessive activity. As such, the reduction in the level and/or activity of such gene products would bring about the amelioration of disorder symptoms. As discussed above, a high level of circulating LDL and oxidation of LDL play a major role in the pathogenesis of cardiovascular disorders. In one embodiment, the invention provides the use of a compound that reduce the level and/or activity of a target gene product that is involved in the formation and accumulation of LDL, e.g., target genes involved in the synthesis of apoproteins that are constituents of LDL, as well as enzymes and carriers that process and transport lipids, such as cholesterol. In another embodiment, the invention provides the use of a compound that reduce the level and/or activity of a target gene product that is involved in the formation and accumulation of oxidized LDL.

In other aspects, cardiovascular disorders are brought about, at least in part, by the absence or reduction of the level of gene expression, or a reduction in the level of a gene product's activity. As such, an increase in the level of gene expression and/or the activity of such gene products would bring about the amelioration of cardiovascular disorder symptoms.

In some cases, the up-regulation of a gene in a disorder reflects a protective role for that gene product in responding to the condition of the disorder. Enhancement of such a target gene's expression, or the activity of the target gene product, will reinforce the protective effect it exerts. Some cardiovascular disorders may result from an abnormally low level of activity of such a protective gene. In these cases also, an increase in the level of gene expression and/or the activity of such gene products would bring about the amelioration of cardiovascular disorder symptoms.

In one embodiment, the disorder to be treated or prevented by the methods of the invention is atherosclerosis. In humans, this is caused, at least in part, by excessive oxidized LDL. Oxidized LDL is recognized by different types of receptors than native LDL, including scavenger receptors on macrophages. These macrophages can then develop into foam cells which are involved in the etiology of atherosclerosis.

For example, compounds such as those identified through assays described above (e.g., Section 5.4) which exhibit inhibitory activity, may be used in accordance with the invention to treat or prevent cardiovascular disorder or ameliorate the symptoms. Such molecules may include, but are not limited to small organic molecules, peptides, antibodies, and the like.

The agents or compounds identified by the methods of the invention can be used for treatment or prevention of disease. In certain embodiments, compounds identified by the methods of the invention can be used to treat cholestasis. In related embodiments, the cholestatic disease is the result of a heritable genetic defect. In other embodiments, the cholestatic disease is acquired. Cholestatic disease is characterized by an impairment of bile flow (van Mil et al., 2001, Seminars in Liver Disease 21:4).

In certain embodiments, compounds identified by the methods of the invention can also be used to increase reactive oxygen species (ROS) levels by administration of such compounds to an organism in need thereof or as a prophylactic.

In certain embodiments, the compounds identified by the methods of the invention can be used to modulate cholesterol levels in a patient in need of having an adjustment of the level of cholesterol in circulation. In other embodiments, the compounds identified by the methods of the invention can be used to prevent increases in cholesterol levels in a patient. In certain embodiments, the compounds identified by the methods of the invention can modulate cholesterol metabolism.

In certain embodiments, the compounds identified by the methods of the invention can be used to treat or prevent diseases involve the buildup of lipid and/or fat deposits in blood vessels, such as arteriosclerosis or atherosclerosis. In other embodiments, the compounds identified by the methods of the invention can be used to prevent arteriosclerosis or atherosclerosis.

5.5.1 Determination of Therapeutic/Prophylactic Utility

The methods and compositions of the invention are preferably tested in vitro, and then in vivo, for the desired therapeutic or prophylactic activity, prior to use in humans. For example, in vitro assays which can be used to determine whether administration of a specific therapeutic method is indicated, include in vitro cell culture assays in which a patient tissue sample is grown in culture, and exposed to or otherwise administered a therapeutic agent, and the effect of such agent upon the tissue sample is observed. Alternatively, instead of culturing cells from a patient, agents and methods may be screened using cells of a relevant cell line (e.g. endothelial cell line).

Agents for use in therapy can be tested in suitable animal model systems prior to testing in humans, including but not limited to in rats, mice, chicken, cows, monkeys, rabbits, hamsters, etc. The agents can then be used in the appropriate clinical trials.

Toxicity and efficacy of the prophylactic and/or therapeutic methods of the instant invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.5.2 Combination Therapy with Other Prophylactic/Therapeutic Agents

In some embodiments, the invention provides methods for treating a disorder by administering one or more agents of the invention in combination with another prophylactic/therapeutic agent. In some specific embodiments, dosages of the other prophylactic/therapeutic can be reduced due to combination therapy with agents of the invention such that the prophylactic/therapeutic agents can be administered less frequently or unwanted/adverse effects are reduced. The invention also encompasses synergistic combinations where the efficacy of the combination prophylactics/therapeutics is greater than additive. In certain embodiments, the combination prophylactics/therapeutics encompassed by the invention provide an improved overall therapy relative to administration of any component alone.

The present invention also relates to a method for increasing a patient's sensitivity to a prophylactic/therapeutic modality comprising administering an agent of the invention (e.g., MOLL nucleic acid, MOLL polypeptide, MOLL agonist, MOLL antagonist, inhibitor of a MOLL agonist, inhibitor of a MOLL antagonist) to a subject who is receiving, had received or will receive the prophylactic/therapeutic modality. In a specific embodiment, the patient had been refractory to one or more other non-MOLL based prophylactics/therapeutics.

Examples of prophylactic/therapeutic agents that can be used in combination are bile-acid-binding resins (e.g., cholestyramine and colestipol hydrochloride), statins (e.g., lovastatin and pravastatin), fibrates (e.g., clofibrate), and niacin. Prophylactic/therapeutic agents and their dosages, routes of administration and recommended usage are known in the art and have been described in such literature as the Physician's Desk Reference (56^{th} ed., 2002), and Goodman & Gilman's The Pharmacological Basis of Therapeutics, Tenth Edition, Chapter 36. Drug Therapy for Hypercholesterolemia and Dyslipidemia, which are incorporated herein by reference in their entireties.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second prophylactic or therapeutic agent to a subject which had, has, or is susceptible to a disorder. Any additional prophylactic or therapeutic agent can be administered in any order with the other additional prophylactic or therapeutic agents. In certain embodiments, an agent of the invention is one of the prophylactic and/or therapeutic agents administered. In certain embodiments, agent of the invention is administered in combination with a prophylactic and/or therapeutic agents that is not based on a MOLL polypeptide of the invention.

### 5.6 Pharmaceutical Compositions

The compositions of the invention include bulk drug compositions useful in the manufacture of pharmaceutical compositions (e.g., impure or non-sterile compositions) and parenteral pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more agents of the invention and a pharmaceutically acceptable carrier. In a further embodiment, the composition of the invention further comprises an additional prophylactic or therapeutic useful for treating, managing, or preventing the same disorder as the agent of the invention.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infuision, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

5.6.1 Modes of Administration

Pharmaceutical compositions for use in accordance with the present invention may be formulated in conventional manner using one or more physiologically acceptable carriers or excipients. The formulation should suit the mode of administration. Various delivery systems are known and can be used to administer an agent of the invention or the combination of an agent of the invention and a prophylactic or therapeutic useful for treating, managing, or preventing the same disorder as the agent of the invention. Administration of the pharmaceutical compositions of the invention includes, but is not limited to, oral, inhalation, parenteral, intravenous, intramuscular, intraperitoneal, intraorbital, intraocular, intracapsular, intraspinal, intrasternal, intra-arterial, intradermal, subcutaneous, topical, depo injection, implantation, time-release mode, intracavitary, intranasal, intratumor, and controlled release, transmucosal, and rectal administration. The skilled artisan can appreciate the specific advantages and disadvantages to be considered in choosing a mode of administration.

Multiple modes of administration are encompassed by the invention. For example, a agent of the invention is administered by subcutaneous injection, whereas a combination therapeutic agent is administered by intravenous infusion.

Systemic administration can also be by transmucosal or transdermal means. For transmucosal or transdermal administration, penetrants appropriate to the barrier to be permeated are used in the formulation. Penetrants for transmucosal administration are generally known in the art, and include, for example, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Pharmaceutical compositions adapted for transdermal administration can be provided as discrete patches intended to remain in intimate contact with the epidermis for a prolonged period of time.

Pharmaceutical compositions adapted for topical administration to the eye include, for example, eye drops or injectable compositions. In these compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilles and mouthwashes.

Pharmaceutical compositions adapted for oral administration may be provided, for example, as capsules, tablets, powders, granules, solutions, syrups, suspensions (in aqueous or non-aqueous liquids), edible foams, whips, or emulsions. Tablets or hard gelatine capsules may comprise, for example, lactose, starch or derivatives thereof, magnesium stearate, sodium saccharine, cellulose, magnesium carbonate, stearic acid or salts thereof. Soft gelatin capsules may comprise, for example, vegetable oils, waxes, fats, semi-solid, or liquid polyols. Solutions and syrups may comprise, for example, water, polyols and sugars.

Pharmaceutically compatible binding agents, and/or adjuvant materials can be included as part of the composition. The tablets, pills, capsules, and troches can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

An active agent intended for oral administration may be coated with or admixed with a material (e.g., glyceryl monostearate or glyceryl distearate) that delays disintegration or affects absorption of the active agent in the gastrointestinal tract. Thus, for example, the sustained release of an active agent may be achieved over many hours and, if necessary, the active agent can be protected from being degraded within the gastrointestinal tract. Taking advantage of the various pH and enzymatic conditions along the gastrointestinal tract, pharmaceutical compositions for oral administration may be formulated to facilitate release of an active agent at a particular gastrointestinal location. Oral formulations preferably comprise 10% to 95% active ingredient by weight.

Pharmaceutical compositions adapted for nasal administration can comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers such as, for example, nasal sprays or nasal drops. These compositions may comprise aqueous or oil solutions of the active ingredient. Compositions for administration by inhalation may be supplied in specially adapted devices including, but not limited to, pressurized aerosols, nebulizers, or insufflators, which can be constructed so as to provide predetermined dosages of the active ingredient.

Pharmaceutical compositions adapted for rectal administration can be prepared in the form of suppositories (e.g., with conventional suppository bases such as cocoa butter and other glycerides) or retention enemas for rectal delivery. Pharmaceutical compositions adapted for vaginal administration may be provided, for example, as pessaries, tampons, creams, gels, pastes, foams, or spray formulations.

In one embodiment, a pharmaceutical composition of the invention is delivered by a controlled-release system. Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760. For example, the pharmaceutical composition may be administered using intravenous infusion, an implantable osmotic pump, a transdermal patch, liposomes, or other modes of administration. In one embodiment, a pump may be used (See, e.g., Langer, 1990, Science 249:1527-33; Sefton, 1987, CRC Crit. Ref. Biomed. Eng. 14:201; Buchwald et al., 1980, Surgery 88:507; Saudek et al., 1989, N. Eng. J. Med. 321:574). In another embodiment, the compound can be delivered in a vesicle, in particular a liposome (See, e.g., Langer, 1990, Science 249:1527-1533; Treat et al., 1989, in Liposomes in the Therapy of Infectious Disease and Cancer, Lopez-Berestein and Fidler (eds.) Liss, N.Y., pp. 353-65; Lopez-Berestein, ibid., pp. 317-27; International Patent Publication No. WO 91/04014; U.S. Pat. No. 4,704,355). In another embodiment, polymeric materials can be used (See, e.g., Medical Applications of Controlled Release, Langer and Wise (eds.) CRC Press: Boca Raton, Fla., 1974; Controlled Drug Bioavailability, Drug Product Design and Performance, Smolen and Ball (eds.) Wiley: New York (1984); Ranger and Peppas, 1953, J. Macromol. Sci. Rev. Macromol. Chem. 23:61; Levy et al., 1985, Science 228:190; During et al., 1989, Ann Neurol. 25:351; Howard et al., 1989, J. Neurosurg. 71:105).

In one embodiment, the active compounds, which comprise polynucleotides, polypeptides, antibodies, or other agents of the invention, are prepared with carriers that will protect the compound from rapid elimination from the body. Such carriers can be a controlled release formulation, which includes, but is not limited to, implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In a particular embodiment, polypeptides of the invention can be administered using a biodegradable polymer having reverse thermal gelatin properties (See, e.g., U.S. Pat. No. 5,702,717).

In yet another embodiment, a controlled release system can be placed in proximity of the target. For example, to treat cancer, a micropump may deliver controlled doses directly into the tumor region, thereby requiring only a fraction of the systemic dose (See, e.g., Goodson, 1984, in Medical Applications of Controlled Release, vol. 2, pp. 115-138).

In one embodiment, it may be desirable to administer a pharmaceutical composition of the invention locally to the area in need of treatment; this may be achieved, for example, by local infusion during angioplasty, surgery, topical application (e.g., in conjunction with a wound dressing after surgery), injection, by means of a catheter, by means of a suppository, or by means of an implant. An implant can be of a porous, non-porous, or gelatinous material, including membranes, such as sialastic membranes, or fibers.

Pharmaceutical compositions suitable for injectable use include sterile aqueous solutions, or dispersions, or sterile powders (for the extemporaneous preparation of sterile injectable solutions or dispersions). For intravenous administration, suitable carriers include physiological saline, bacteriostatic water, Cremophor EL™ (BASF; Parsippany, N.J.) or phosphate buffered saline (PBS). The carrier can be a solvent or dispersion medium comprising, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, or by the use of a surfactant. Prevention of the action of microorganisms can be achieved by various antibacterial and antifingal agents, such as for example, parabens, chlorobutanol, phenol, ascorbic acid, and thimerosal. It can be preferable to include in the composition isotonic agents, such as for example, sugars, polyalcohols (e.g., mannitol), sorbitol, and sodium chloride. Prolonged absorption of the injectable compositions can be brought about by including in the composition an agent which delays absorption, such as for example, aluminum monostearate and gelatin.

For administration by inhalation, the compounds are delivered in the form of an aerosol spray from a pressurized container or dispenser which comprises a suitable propellant, e.g., a gas such as carbon dioxide, or a nebulizer.

Oral or parenteral compositions can be formulated in dosage unit form for ease of administration and uniformity of dosage. Dosage unit form as used herein refers to physically discrete units suited as unitary dosages for the subject to be treated, such that each unit contains a predetermined quantity of active compound, which is calculated to produce the desired therapeutic effect, and a pharmaceutical carrier. The skilled artisan will appreciate that dosage unit forms are dependent on the unique characteristics of the active compound, the particular therapeutic effect to be achieved, and the limitations inherent in the art of compounding such an active compound for human administration.

The skilled artisan will appreciate that certain factors may influence the dose necessary to effectively treat a subject, which factors include, but are not limited to, previous treatment regimens, severity of the disorder, general health and/or age of the subject, and concurrent disorders. Moreover, treatment of a subject with a therapeutically effective amount of an agent of the invention can include a single treatment or, preferably, can include a series of treatments.

5.6.2 Administration of Nucleic Acids as Agents of the Invention

In a specific embodiment, nucleic acids of the invention (e.g., MOLL antisense nucleic acids, MOLL dsRNA, or nucleic acids that encode a MOLL polypeptide or MOLL intrabody) are administered to treat, prevent or manage a disorder by way of gene therapy. Gene therapy refers to therapy performed by the administration to a subject of an expressed or expressible nucleic acid. In this embodiment of the invention, the nucleic acids mediate a prophylactic or therapeutic effect.

Any of the methods for gene therapy available in the art can be used according to the present invention. Exemplary methods are described below.

For general reviews of the methods of gene therapy, see Goldspiel et al, 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11:155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises a nucleic acid of the invention (e.g., encode an antisense or intrabody molecule), said nucleic acid being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules used comprise nucleic acid molecules of the invention flanked by regions that promote homologous recombination at a desired site in the genome, thus providing for intrachromosomal expression of the nucleic acids of the invention (Koller and Smithies, 1989, PNAS 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. In a specific embodiment, the nucleic acid sequences are directly administered in vivo. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see e.g., U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide, e.g., through a thioester bond, which is known to enter the cell (e.g., a membrane permeable sequence) and/or nucleus, by administering it in linkage to a ligand subject to receptor-mediated endocytosis (see, e.g., Wu and Wu, 1987, J. Biol. Chem. 262:4429) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; W092/203 16; W093/14188, WO 93/20221). Alternatively, the nucleic acid can be introduced intracellularly and incorporated within host cell DNA for expression, by homologous recombination (Koller and Smithies, 1989, PNAS 86:8932; and Zijlstra et al., 1989, Nature 342:435).

In a specific embodiment, viral vectors that contain the nucleic acid sequences of the invention are used. For example, a retroviral vector can be used (see Miller et al., 1993, Meth. Enzymol. 217:581). These retroviral vectors contain the components necessary for the correct packaging of the viral genome and integration into the host cell DNA. The nucleic acid sequences to be used in gene therapy are cloned into one or more vectors, which facilitates delivery of the nucleic acid into a subject. More detail about retroviral vectors can be found in Boesen et al., 1994, Biotherapy 6:291-302, Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431; Rosenfeld et al., 1992, Cell 68:143; Mastrangeli et al., 1993, J. Clin. Invest. 91:225; International Patent Publication No. WO94/12649; and Wang et al., 1995, Gene Therapy 2:775. In a preferred embodiment, adenovirus vectors are used. Adeno-associated virus (AAV) has also been proposed for use in gene therapy (Walsh et al., 1993, Proc. Soc. Exp. Biol. Med. 204:289-300; and U.S. Pat. No. 5,436,146).

Numerous techniques are known in the art for the introduction of foreign genes into cells (see, e.g., Loeffler and Behr, 1993, Meth. Enzymol. 217:599; Cohen et al., 1993, Meth. Enzymol. 217:618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

### 6. EXAMPLES

### Identification of Dsc Mutants

### 6.1 Materials and Methods

6.1.1 General Methods and Strains

Most strains were derived from the wild-type C. elegans N2 Bristol strain and were cultured as described (Brenner, 1974, Genetics 77:71-94). The wild-type RW7000 strain was used for some of the linkage analyzes, using sequence-tagged sites (STS). All animals were grown at 20° C. unless otherwise indicated. The genes, alleles, and STSs used are as follows:

LGI: b1i-4(e937), stP124; LGII: ro1-6(e187), ro1-1(e91), unc-52(su250ts), maP1; LGIII: daf-2(e1368), dpy-17(e164), clk-1(qm30), unc-32(e189), dec-7(sa296), vab-7(e1562); LGIV: flr-3(ut9), dpy-9(e12), unc-33(e204), unc-5(e53), unc-31(e928), dpy-4(e166sd), sP4; LGV: unc-34(e315), dpy-11(e224), stP192; LGX: lin-15(n765), flr-4(ut7), unc-3(e151), lon-2(e678), unc-2(e55), stP103.

6.1.2 Isolation of Suppressor Mutations clk-1(qm30) animals were mutagenized with 25 mM ethyl methane sulfonate (EMS), as described (Sulston and Hodgkin, 1988, Methods, pp. 587-606 in The Nematode Caenorhabditis elegans, edited by W. B. Wood. Cold Spring Harbor Laboratory, Cold Spring Harbor, N.Y.). Groups of five mutagenized hermaphrodites (P0) were plated on 60-mm petri dishes and left to self-fertilize. Groups of 25 F1 animals were transferred to 90-mm plates as young adults and were left to lay eggs for ˜24 hours. F2 animals were scored for one defecation cycle each at 20° C. Based on the assumption that specific suppressor mutations would not cause morphological or other behavioral defects, only wild-type looking animals were scored. A maximum of 50 F2 animals were scored from each plate to minimize the probability of scoring multiple worms carrying the same mutation. Animals that had a defecation cycle length of less than 65 seconds were picked to 60-mm plates, singled, and left to self-fertilize. The progeny (F3) of the singled candidate worms were scored for defecation; only those strains that had a significant proportion of fast defecating worms in the F3 generation were kept for further genetic and phenotypic analysis. A total of 5421 F2 animals were screened, a number equivalent to 2134 haploid genomes (Ellis and Horvitz, 1991, Development 112:591-603).

6.1.3 Mapping of Suppressor Mutations

Mutations were linked to chromosomes in one of three ways. For mutations that could be mapped on wild-type (clk-1(+)) backgrounds, linkage analysis was performed either by using the DA438 strain (for qm133, qm141) (Avery, 1993, Genetics 133: 897-917), or by using the wild-type RW7000 strain and testing linkage to STSs by PCR analysis (for qm142, qm183) as described (Williams, 1995, Genetic Mapping with Polymorphic Sequence-Tagged Sites, pp. 81-95 in Caenorhabditis elegans: Modern Biological analysis of an Organism, edited by H. F. Epstein and D. C. Shakes. Academic Press, San Diego). For mutations that had less obvious phenotypes on wild-type backgrounds or that could not be linked to an STS, linkage to each chromosome was tested separately, using strains that contained visible marker mutations in the clk-1(qm30) background (for qm178, qm179, qm180, qm182, and qm184). Formal linkage analysis was not needed for qm166, as it was found to be tightly linked to clk-1(qm30). Once linkage to a chromosome was established, mutations were mapped more precisely using 2- and 3-point mapping strategies.

To avoid marker effects, for most 3-point mapping experiments, homozygous recombinant progeny were isolated from F2 recombinant animals, and were crossed with homozygous mutant males. Defecation was scored in the F1 cross-progeny. Because qm133 has other phenotypes that could be scored in addition to defecation cycle length, including a thin, starved appearance, all mapping of qm133 was done using conventional methods (by singling recombinant F2 animals and scoring for the presence of ½ qm133 animals in the F3 generation). All mapping of qm133, qm141, and qm183 was done with strains in a clk-1(+) background and was scored at 20° C.; all mapping of qm179, qm180, qm182, and qm184 was done with strains in a clk-1(qm30) background, and was scored at 25° C. Mapping of qm142 was also done in a clk-1(qm30) background, but was scored at 20° C. As qm142 has a dominant effect, it was mapped by crossing homozygous recombinant progeny with clk-1(qm30) males and scoring the F1 animals after 48hr after they had reached adulthood, when the dominant effect of qm142 is strongest. The mapping data is summarized in Table 2.

6.1.4 Complementation Tests

Complementation tests were performed when different mutations appeared to map to the same genetic region, or when a mutation mapped to a region where a dec gene had previously been mapped. Generally, males homozygous for one mutation were mated to hermaphrodites homozygous for the other mutation, and defecation was scored in the trans-heterozygous F1 animals. In this manner it was found that: qm166 and qm178 fail to complement each other, and the previously identified mutant dec-7(sa296), which are therefore all likely to be allelic. Similarly, qm179, qm184 and qm180all fail to complement each other, and are therefore all likely to be allelic. qm133 maps in the region of flr-4, but complements flr-4(ut7), suggesting that they define distinct genes. Similarly, qm182 maps in the region of flr-3, but complements flr-3(ut9), suggesting that they define distinct genes.

6.1.5 Behavioral Analyzes

Defecation was scored in hermaphrodites on their first day of adulthood at 20° C., unless otherwise indicated. The defecation cycle length was defined as the duration between the pBoc steps of two consecutive defecations. Each animal was scored for five consecutive cycles (six consecutive pBocs), and the mean and standard deviation was calculated. To prevent the animals from being heated by the microscope lamp during the scoring session, the plates were placed on ‘heat sinks’ (petri dishes filled with water) and animals were only scored from them for a maximum of 15 minutes.

6.1.6 Temperature Shift Experiments

Animals were grown at 20° C. and transferred to either 15° C. or 25° C. as young adults. Animals were then scored at 15° C. or 25° C. 2-6 hours after being transferred to that temperature. As it was difficult to maintain the temperature of the plates at 15°, animals were only scored for three consecutive cycles at this temperature, and plates were only kept on the microscope for as long as was required to score one animal.

6.1.7 qm142 Time Course Studies

To generate heterozygous dsc-2(qm142)/+animals, dsc-2(qm142) dpy-11(e224) animals were mated with N2 males. To generate qm30; qm142/+animals, clk-1(qm30); unc-5(e53) hermaphrodites were mated with clk-1(qm30); dsc-2(qm142) males. Late L4 stage, F1 generation animals were picked to plates and examined 3 hours later. Animals that had molted to adults during this period were used for the experiment and were considered to be 1.5 hr old adults at the end of the interval. The sample size of each genotype for each time point is ˜10. The same sets of animals were scored at the different time points.

6.1.8 Statistical Analyses

A 2-sample student's t-tests was performed taking into account the unequal variances of the samples to determine if N2 and clk-1(qm30) mutants were different when grown and scored at 15° C. and 25° C. than when they were grown and scored at 20° C. Highly significant differences (p<0.05) were found for both N2 and clk-1(qm30) at 20° C. vs at 15° C. Significant differences were not detected for N2 at 20° C. vs at 25° C. (p=0.40) or for clk-1(qm30) at 20° C. vs at 25° C. (p=0.27). A 2-sample student's t-tests was also performed to determine if N2 and clk-1(qm30) mutants were different when grown at 20° C. and scored at 15° C. and 25° C. than when they were grown and scored at 20°. Highly significant differences (p<0.001) were found all comparisons except for clk-1(qm30) at 20° C. vs at 15° C. (p=0.29) and at 20° C. vs at 25° C. (p=0.99). A 2-sample student's t-tests was also performed to determine if each suppressor mutation had a significant effect on the defecation cycle by comparing, at every temperature, each clk-1(qm30) double mutant strain with clk-1(qm30), and every clk-1(+) mutant strain with clk-1(+). Highly significant differences (generally p<0.001) were found for all comparisons except: clk-1(qm30) dec-7(qm166) vs clk-1(qm30) at 15° (p=0.34) and at 25° C. (p=0.97); clk-1(qm30) dec-7(qm178) vs clk-1(qm30) at 15° C. (p=0.83) and at 25° C. (p=0.15); clk-1(qm30); dsc-3(qm184) vs clk-1(qm30) at 15° C. (p=0.80); clk-1(qm30); dec(qm183) vs clk-1(qm30) at 20° C. (p=0.12) and at 25° C. (p=0.31).

6.2 Defecation Cycle Phenotype of Clk-1 Mutants

Defecation in Caenorhabditis elegans is achieved by the periodic activation of a stereotyped motor program. In wild-type animals, the defecation cycle length is 56 seconds, with a standard deviation of only 3.4 sec (at 20° C.). As previously described (Felkai et al., 1999, EMBO J 18: 1783-1792 and Wong et al., 1995, Genetics 139: 1247-1259), in clk-1 mutants the defecation cycle is both increased in length and more irregular: in clk-1(qm30) animals, the cycle length is 88 sec, with a standard deviation of 14 sec, and in the weaker allele clk-1(e2519), it is 77 sec, with a standard deviation of 7 sec (at 20° C.) (Table 1).

To examine the effect of temperature on the defecation cycle length of wild-type and clk-1 mutant worms, worms were raised for two generations at 15° C. or 25° C. and scored at the temperature at which they had been raised. clk-1 mutants were slower than the wild type at all temperatures and both genotypes had significantly longer cycles when grown and scored at 15° C. than when grown and scored at 20° C. (data not shown). In contrast, when wild-type or mutant worms are grown and scored at 25° C. the defecation rates were not significantly different from those at 20° C. (data not shown).

The effect of temperature shifts on the defecation cycle length was examined in wild-type and clk-1 worms. Wild type worms transferred from 20° C. to 25° C. had a mean cycle length that was decreased by 13 sec while wild type worms transferred from 20° C. to 15° C. had a mean cycle length increased by 26 sec. This adjustment happened very rapidly, e.g., wild-type worms scored 5 minutes or 2-6 hours after they were transferred to 25° C. had substantially similar decreased cycle length (data not shown). Note that the defecation cycle of wild-type worms raised and scored at 25° C. is in fact slower than when the worms are raised at 20° C. and then shifted to 25° C. for scoring. This suggested that the worms had become adapted or acclimated to the higher temperature during development and were thus less affected when scored at that temperature. However, whether the worms are raised at 15° C. or 20° C. did not affect the length of the defecation cycle when scored at 15° C.

In contrast to the observations with the wild type worms, clk-1 mutants were unable to re-adjust the length of their defecation cycle after being shifted to a new temperature. When clk-1 mutants were transferred from either 20° C. to 25° C. or from 20° C. to 15° C. there was no change in the mean cycle length (Table 1). This suggested that mutants adapted to 20° C. cannot re-adjust the defecation cycle length upon a temperature shift, thus, wild type clk-1 activity was required for the adjustment to occur.

6.3 Suppression Screen Slow Defecation Phenotype

A screen for suppressors of clk-1 was conducted in order to identify genes that interact with clk-1 to regulate the defecation cycle length. Mutations isolated were those that could suppress clk-1 and restore the length of the defecation cycle to that of wild type worms. clk-1 (qm30) worms were mutagenized with EMS and second generation (F2) animals were directly scored for one defecation cycle each at 20° C. Animals that had a cycle length of less than 65 sec were kept for further analysis. In this manner, 5421 F2 animals (an equivalent of ˜2134 haploid genomes) were screened and eight suppressor mutations were identified. Seven of these mutations were recessive and one mutation, dsc (qm142), had dominant effects (described in detail below). Based on the mapping and complementation tests performed (Table 2), these mutations defined four new complementation groups which have been called dsc for defecation suppressor of clk-1. Two mutations, qm166 and qm178, were alleles of the previously identified Dec-s gene dec-7. Two other recessive mutations were isolated from the screen, dsc (qm141) and dsc (qm183), affected defecation but did not suppress clk-1(qm30).

### 6.4 Analysis of Dsc Suppressors

The phenotypes of the suppressor mutants were analyzed in a number of different ways. All of the mutations identified in the screen were re-isolated on a wild-type clk-1 background and a clk-1 (e2519), an allele with partial clk-1 activity, background. The length of the defecation cycle was scored in the dsc single mutants and dsc/clk-1 double mutants. Such scoring was conducted at 20° C. as well as after shits to 15° C. and 25° C. Results are shown in Table 1.

6.4.1 Effect in a clk-1(qm13O) or Wild Type Background

The suppressors fell into two distinct classes based on their differential abilities to suppress clk-1(qm30) after temperature shifts (particularly to 25° C.). Class I mutants strongly suppress clk-1(qm30) at 20° C. as well as after shifts to 25° C. Isolated mutants which fell into this class were dsc-3(qm179, qm180, qm184) and dsc-4(qm182). At 25° C., dsc-3(qm179) the strongest mutant in this respect, shortened the defecation cycle length of clk-1(qm30) mutants to less than the wild type length. Thus, the profile of defecation in the Class I dsc/clk-1(qm30) double mutants at the different temperatures is substantially similar to that of the wild type (i.e., slowest at 15° C., fastest at 25° C.). Therefore, these mutants suppressed the long defecation cycle of clk-1(qm30) mutants when grown and scored at 20° C., as well as the temperature insensitivity when grown at 20° C. and then shifted to another temperature.

In contrast to Class I mutants, the Class II mutants suppressed only weakly (dsc-1(qm133) and dsc-2(qm142)) or not at all (dec-7(qm166, qm178)), after a temperature shift. This suggested that, although these mutants suppressed the long defecation cycle of clk-1(qm30) mutants at 20° C., they cannot suppress the inability of clk-1(qm30) mutants to readjust their defecation cycle length after a temperature shift.

The phenotypes of Class I and Class II mutants were much more similar on the wild-type background than on the clk-1(qm30) background, although the Class I mutants generally had weaker effects on the wild type than the Class II mutants, particularly at 15° C. and 20° C. All the mutations significantly decreased the length of the defecation cycle at all temperatures and both the Class I and Class II mutants reacted to changes in temperature like the wild type (i.e. defecation was fastest at 25° C. and slowest at 15° C.). This indicates that the lack of effect of the Class II mutants at 25° C. on the clk-1(qm30) background cannot be due to any of the dsc mutations being intrinsically temperature sensitive. Overall, the observation that on the clk-1(qm30) background there are two distinct classes of interactions suggests that the genes of the different classes interact with clk-1 in different ways.

6.4.2 Effect in a clk-1 (e2519) Background

The dsc mutations were re-isolated on the background of clk-1(e2519), which has a weaker phenotype than clk-1(qm30). Although the defecation cycle lengths of the clk-1(e2519)/dsc double mutants were shorter than that of the clk-1(qm30)/dsc double mutants, the differences were generally very small (Table 1; see below for a description of the special case of dsc-5(qm14l)).

While screening for suppressors, a number of candidate strains were isolated from which mutations that strongly suppress clk-1(qm30) could not be isolated. In two cases, however, the candidate strains did segregate mutations that significantly affected the rate of defecation. One of these mutations, dsc-5(qm14l), had a strong effect on the defecation cycle of wild-type worms at all temperatures, as well as on clk-1(e2519) mutants, but only a very weak, but significant, effect on clk-1(qm30) mutants at all temperatures. This suggests that dsc-5(qm141) might be acting largely through clk-1, as it affects the null mutant (qm30) only slightly, whereas it fully suppressed the partial loss of function mutation clk-1 (e2519). The other mutation isolated, qm183, also had significant effects on the defecation cycle of wild-type worms at all temperatures, but could not suppress either clk-1(e2519) or clk-1(qm30) mutants. It is clear, therefore, that the slow defecation of clk-1(qm30) mutants cannot simply be suppressed by every mutation that decreases the defecation cycle length of the wild type. In fact, some mutations may require full (i.e. qm183) or at least partial (i.e. qm141) wild type clk-1 activity in order to affect the defecation cycle.

6.4.3 Further Characterization of dsc-2 and dec-7

The strength of the qm142 mutant phenotype changes with the age of the animal, in both heterozygotes and homozygotes, albeit at very different rates. A time course study was performed defecation was scored in the same animals at different time points after they had molted to adults. In a clk-1(qm30) background, the homozygous dsc-2(qm142) animals are almost as slow as the clk-1(qm30) animals 2 hours after molting to adults, but by 8 hr, the defecation cycle length has been restored to the wild-type length, and by 18 hr, the defecation cycle length is significantly shorter than that of the wild type. The defecation cycle length of clk-1(qm30) worm with one dsc-2(qm142) allele and one wild type dsc-2 allele was very similar to that of clk-1(qm30) animals until about 40 hr. By about 48 hours after molting to adults, the dsc-2(qm142)/+heterozygous animals had defecation cycle lengths that were as fast as the age matched wild-type animals. As the dsc-2(qm142)/+heterozygotes never become as fast as the homozygotes at any time point, and take longer than the homozygotes to speed up significantly, the effect of the qm142 mutation is incompletely dominant over the wild-type allele. This was also confirmed by observations of qm142 heterozygotes and homozygotes on the wild-type background, although the effects were much less dramatic. One way in which the dsc-2(qm142) allele could have this semi-dominant time-dependent effect is that the mutation results in a protein that can interfere with the function of the wild-type dsc-2 protein. An accumulation of the mutant product with time could increase the severity of the mutant phenotype.

All the suppressor mutants were characterized by analyzing the mean defecation cycle length of a number of animals that had each been scored for five defecation cycles (Table 1). Animals carrying dec-7 mutations in a clk-1(qm30) background had very high standard deviations at 15° C. and 20° C. but not at 25° C. This variability was analyzed further by plotting the frequency of single defecation cycle lengths of clk-1/dec-7 animals at four different temperatures. At all temperatures, there was only one frequency peak for clk-1(qm30) mutants, but there were two peaks for both clk-1/dec-7 double mutant strains at the three temperatures below 25° C. (e.g., 22.5° C., 20° C., and 15° C.). One of the peaks occurs at a cycle length that is two times that of where the other peak occurs. At 25° C., however, there is only one peak, which coincides with the clk-1(qm30) peak.

One interpretation of this pattern is that clk-1 and/or dec-7 have a role in coupling the activation of the defecation motor program (DMP) to the cycle, such that the coupling increasingly fails in clk-1/dec-7 double mutants with increasing temperature. This would result in double cycle lengths and could mean that at 25° C. every cycle observed is actually a double cycle. Multiple discrete cycle lengths are not observed in dec-7 mutants on a wild type clk-1 background or in other Class II mutants, and thus, this phenomenon appears to be specific to clk-1/dec-7 mutants.

Another interpretation is that, with increasing temperature, there is a decrease in the penetrance of the suppression of clk-1 by dec-7. This is suggested by the observation that at 20° C., 22.5° C., and 25° C. the main peak coincides with the unsuppressed clk-1 peak. However, this does not happen at 15° C., which is difficult to explain with this interpretation.

TABLE 1
Quantitative phenotypic analysis of suppressor mutations on wild-type and clk-1 backgrounds.

| | Length of Defecation Cycle(sec) | | | | | | |
|---|---|---|---|---|---|---|---|
| | Wild type | | | clk-1(qm30) | | | clk-1(e2519) |
| Mutation | 15° | 20° | 25° | 15° | 20° | 25° | 20° |
| + | 82.8 ± 9.2 | 55.9 ± 3.4 | 42.5 ± 2.5 | 90.7 ± 6.0 | 88.3 ± 13.5 | 88.4 ± 13.8 | 77.1 ± 6.7 |
| dsc-1(qm133) | 62.3 ± 9.6 | 46.9 ± 4.5 | 33.4 ± 4.0 | 76.7 ± 10.6 | 64.7 ± 12.2 | 72.9 ± 18.4 | 62.2 ± 12.9 |
| dsc-2(qm142sd) | 61.1 ± 5.0 | 45.1 ± 15.5 | 31.2 ± 3.7 | 76.7 ± 21.1 | 60.0 ± 14.0 | 74.6 ± 10.0 | 58.8 ± 6.7 |
| dec-7(qm166) | 55.1 ± 3.2 | 35.6 ± 3.2 | 34.1 ± 4.7 | 86.2 ± 22.2 | 58.9 ± 27.8 | 88.6 ± 13.2 | ND |
| dec-7(qm178) | ND | ND | ND | 91.8 ± 23.9 | 63.2 ± 24.0 | 92.3 ± 11.4 | ND |
| dsc-3(qm179) | 75.6 ± 4.4 | 51.9 ± 2.8 | 35.3 ± 3.5 | 76.8 ± 9.1 | 63.0 ± 6.2 | 41.6 ± 4.3 | 56.4 ± 3.0 |
| dsc-3(qm180) | 74.4 ± 5.8 | 52.7 ± 2.9 | 33.7 ± 2.9 | 85.7 ± 9.2 | 68.5 ± 6.9 | 60.0 ± 20.7 | 66.7 ± 5.1 |
| dsc-3(qm184) | 73.9 ± 4.7 | 53.0 ± 3.2 | 37.0 ± 2.2 | 90.3 ± 5.3 | 66.0 ± 10.3 | 53.3 ± 7.6 | 61.4 ± 5.7 |
| dsc-4(qm182) | 68.1 ± 5.6 | 47.8 ± 2.5 | 33.9 ± 2.0 | 75.8 ± 7.5 | 60.7 ± 6.3 | 48.0 ± 7.7 | 60.2 ± 3.4 |
| dsc-5(qm141) | 64.7 ± 8.1 | 46.3 ± 7.0 | 36.5 ± 1.8 | 79.5 ± 6.9 | 75.5 ± 9.6 | 74.8 ± 7.4 | 56.7 ± 5.1 |
| dec(qm183) | 69.7 ± 5.2 | 49.9 ± 3.0 | 33.3 ± 1.2 | 102.2 ± 11.6 | 84.8 ± 12.2 | 84.4 ± 13.9 | 83.1 ± 15.8 |

TABLE 2
Summary of genetic mapping of mutants isolated in the suppressor screen.

| 6.5 Mutation | Genetic Mapping Data^{a} |
|---|---|
| dsc-1(qm133) X | [unc-3 lin15/dsc-1] |
| | unc-3 (15/51) dsc-1 (36/51) lin-15 |
| | complements flr-4(ut7) |
| dsc-2(qm142d) V | [clk-1; unc-34 dpy-11/dsc-2] |
| | unc-34 (6/41) dsc-2 (35/41) dpy-11 |
| dsc-3(qm179) IV | [clk-1, unc-33 dpy-4/dsc-3] |
| | unc-33 (17/61) dsc-3 (44/61) dpy-4 |
| | fails to complement qm180 and qm184 |
| dsc-4(qm182) IV | [clk-1; dpy-9/dsc-4] |
| | 0/40^{b} |
| | complements flr-3(ut9) |
| dsc-5(qm141) II | [rol-lunc-52/dsc-5] |
| | rd-1 (7/37) dsc-5 (30/37) unc-52 |
| dec(qm183) X | [lon-2 unc-2/dec] |
| | lon-2 (3/45) dec (42/45) unc-2 |
| dec-7(qm166) III | fails to complement dec-7(sa296) and qm178 |

^{a}The genotypes given in square brackets are those of the F1 animals whose descendants were scored to obtain 2- and 3- factor mapping data.
^{b}Non-Dpy F2 progeny were scored for the presence of the qm182 mutation; the denominator represents the number of qm182 animals that were isolated and the numerator represents the number of qm182 animals that were also heterozygous for the dpy-9 mutation

### 7. EXAMPLES

### Characterization of Dsc-4

### 7.1 Materials and Methods

7.1.1 Strains and Culture Methods

Animals were cultured at 20° C. as described (Brenner, 1974, Genetics 77:71-94) and were fed the E. coli strain OP50 unless otherwise indicated. Cholesterol depletion experiments were performed on standard NGM plates seeded with OP50, but without any added cholesterol (NGM-C). Worms were cultured on NGM-C plates for two or more generations before the rate of germline development was examined.

For RNAi experiments, worms were cultured on NGM plates supplemented with 1 mM IPTG and 50 μg/mL ampicilin, and were fed the E. coli strain HT115 transformed with pPD129.36 derived plasmids. P0s were transferred to the RNAi plates as L4 larvae and the F1 generation was examined.

The wild-type strain was N2 (Bristol strain). The following mutations were used: clk-1(qm30) III; dsc-4(qm182), dpy-9(e12), lin-1(e1026), unc-33(e204), lfe-1/itr-1/dec-4(sy328), unc-24(el38), let-60(n1046) IV, sid-1(qt) V.

7.1.2 Cloning and Sequencing of dsc-4

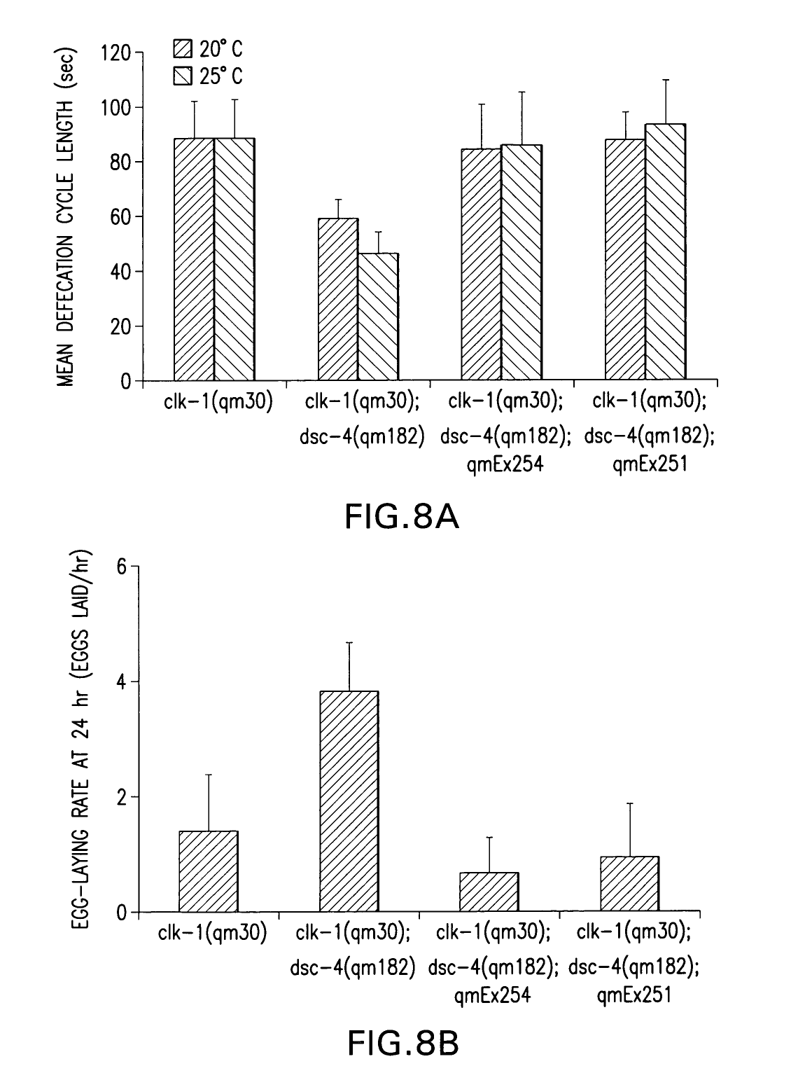

dsc-4 had been mapped to the left arm of LG IV (−27.6). Three-point mapping experiments with unc-33 dpy-9 suggested that dsc-4 was to the left or very close to dpy-9: in a cross between clk-1; dsc-4 and clk-1; unc-33 dpy-9, 14/14 Unc non-Dpy picked up dsc-4, while 0/20 Dpy non-Unc picked up dsc-4. Two-point mapping experiments failed to separate dsc-4 from dpy-9 by recombination, suggesting that dsc-4 is very close to dpy-9, however, as this has also been reported for other genes mapped to the left of dpy-9 (Katsura et al., 1994, Genetics 136:145-154), it is likely that recombination is suppressed in this region. 14 cosmids which roughly correspond to this genetic region were assayed for rescuing activity. They were divided into three pools (pool 1; C15E6, T05C7, B0545, B0312 and F52F6, pool 2; R02D3, T21D12, K02D7, F18F11, AH12, F40D2 and T07A9, pool 3; M04G7 and M04G7 and M02G12) and each pool was injected into clk-1(qm30); dsc-4(qm182) mutants. For each pool, the total concentration of cosmids was 15 μg/mL and the concentration of the co-injection marker Pₜₜₓ₋₃::gfp (Hobert et al., 1997, Neuron 19:345-357) was 185 μg/ml. Pool 2 rescued the fast defecation of clk-1(qm30); dsc-4(qm182) mutants (semicolon indicates mutations on separate chromosome). Each cosmid in pool 2 was tested individually and the cosmid K02D7 was found to rescue the clk-1(qm30); dsc-4(qm182) mutants. PCR products that correspond to the predicted genes on K02D7 were tested individually and it was found that the PCR product corresponding to a gene predicted to be present on the cosmid by Genefinder software and referred to as K02D7.4 (from 26654 to 34896 of K02D7) could rescue the clk-1(qm30); dsc-4(qm182) mutants (FIG. 8A and 8B). This PCR product was amplified from N2 genomic DNA by nested PCR and contains 1.6 kb of the region upstream of dsc-4. PCR products were injected at a concentration of 2 μg/ml with the co-injection marker Pₜₜₓ₋₃::gfp at a concentration of 190 μg/ml.

To determine the nature of the qm182 mutation, the predicted sequence of K02D7.4 was amplified by PCR from genomic DNA samples of clk-1(qm30), clk-1(qm30); dsc-4(qm182) mutants. Both sense and antisense strands of the amplified genomic DNA corresponding to the predicted sequence of K02D7.4 were sequenced. Upon comparison with the predicted nucleotide sequence of K02D7.4, two missense mutations were found in the clk-1(qm30); dsc-4(qm182) mutants (C355T and G605A). A C→T transition at position 354 of the cDNA results in a serine to phenylalanine substitution at position 62 of the protein; A G→A transition at position 605 of the cDNA results in an alanine to threonine substitution at position 146 of the protein (FIG. 4 and 3A). These mutations were absent in the PCR products obtained from clk-1(qm30) mutants.

The dsc-4 DNA sequence shown in FIG. 4 (SEQ ID NO:1) contains 11 exons which can be found following a 5′ untranslated region at nucleotide bases 1-169. The nucleotide base pair locations are as follows: exon 1: 170-215, exon 2: 216-472, exon 3: 473-819, exon 4: 820-924, exon 5: 925-1084, exon 6: 1085-1251, exon 7: 1252-1543, exon 8: 1544-1738, exon 9:1739-2182. exon 10:2183-2529, and exon 11:2530-2848 of SEQ ID NO:1. In the dsc-4 sequence, the exon coding regions are followed by a 3′ untranslated region found at nucleotide base pairs 2849-2922.

The effect of RNAi based on the sequence of dsc-4 on the phenotype of worms with clk-1, dsc-4 mutations and wildtype backgrounds was investigated. RNAi directed against the dsc-4 sequence almost exactly phenocopies the qm182 mutation in the clk-1 background. The defecation rate as well as the egg-laying rate of clk-1(qm30); dsc-4 (RNAi) animals is similar to that of the clk-1(qm30); dsc-4(qm182) mutants. It was observed that the effect of dsc-4 (RNAi) is not additive to that of the dsc-4 mutation for defecation or egg laying in the clk-1 background. Also, dsc-4 (RNAi) in dsc-4 mutants did not cause any obvious enhancement of the phenotype. Together, these results demonstrate that the predicted gene sequence of K02D7.4 corresponds to that of dsc-4, and that the qm182 allele is a strong or complete loss-of-functional allele.

7.1.3 dsc-4 Amino Acid Sequence Analysis

The dsc-4 gene encodes an 892-residue protein, which is similar to the large subunit of the microsomal triglyceride transfer protein (MTP). DSC-4 has a single clear homologue in every animal species whose genome has been sequenced, but appears to be absent from plants and unicellular organisms. The identities between homologues extend across the entire sequence and are not confined to particular regions or domains, and the alignment of DSC-4 with vertebrate MTPs does not require the introduction of numerous gaps (FIG. 3A). When DSC-4 is compared to the NCBI non-redundant protein database using PSI-blast, the 7 proteins with the highest scores are bona fide vertebrate MTPs. The applicants defined amino acid residues 19-295 as the apoB binding domain, amino acid residues 296-609 as the apoB and PDI binding domain and amino acid residues 610-890 as the lipid binding domain. However, these domains are loosely defined functional domains and not protein domains that have characteristic motifs.

7.1.4 Sequencing of the dsc-4 CDNA

A cDNA clone yk357a6 was identified which corresponds to the predicted K02D7.4 gene. The cDNA clone was sequenced. A comparative alignment of the clone sequence and the K02D7.4 sequence showed that the cDNA clone sequence did not contain the full 5′ end of the dsc-4. Using an SLI-specific primer and dsc-4 gene-specific primers, the 5′ end of the dsc-4 was amplified from a first-strand cDNA library generated by the reverse-transcription of poly(A)⁺ selected RNA isolated from mixed-stage wild-type animals using a poly-dT primer. Both sense and antisense DNA strands of cDNA from mixed-stage wild-type animals corresponding to the 5′ end of dsc-4 were sequenced (FIG. 4). The sequence of the dsc-4 cDNA from mixed-stage wild-type animals (SEQ ID NO:1) differed from that predicted for K02D7.4 by Genefinder: the first exon predicted was absent and the second exon was slightly longer at the 5′ end than predicted.

7.1.5 RNA Interference

RNAi experiments were performed as described (Kamath et al., 2001, Genome Biol 2:research0002.1-research0002.10). The controls were fed the HT115 bacteria transformed with the pPD129.36 vector. For all experiments in which the effect of RNA interference with gene action was tested, the controls used for comparison were also cultured on the RNAi plates because both germline and vulval development phenotypes were found to be different on RNAi plates, as compared to normal NGM plates. The dsc-4 RNAi clone was a HindIII-XhoI fragment of yk357a6 cloned into the HindIII XhoI sites of pPD129.36.

7.1.6 Construction of the Transcriptional and Translational dsc-4::gfp Fusions

The translational fusion, Pdsc-4::dsc-4::gfp, was constructed in 2 steps. Step 1: A PCR product containing the region 1.6 kb upstream (from 30259 to 34896 of K02D7) and the 5′-end of dsc-4 was amplified from N2 genomic DNA; a HindIII-XbaI fragment generated from the PCR product was cloned into pPD95.75. Step 2: The 3′ end of dsc-4 (excluding the stop codon) was amplified from yk357a6 (from 498 to 2845); a SaII-StuI fragment generated from the PCR product was cloned into the SaII and SmaI sites of the first clone. The Pdsc-4 ::dsc-4::gfp clone was injected at 100 μg/ml with the co-injection marker Pttx-3::gfp at 100 μg/mL.

The transcriptional fusion, Pdsc-4(1.6kb)::GFP, was constructed as follows: A region containing 1.6 kb upstream and the first 22 amino acid residues of K02D7.4 (from 32017 to 34922 of K02D7) was amplified. The PCR product was cloned into the HindII and SmaI and sites of pPD95.75. The Pdsc-4::GFP was injected at 100 μg/mL with the co-injection marker pRF4 (which contains the dominant mutation rol-6(su1006)) at 100 μg/mL.

7.1.7 Phenotypic Analyses

Time course analysis of egg-laying rate: L4-stage animals were picked to plates and examined three hours later. Animals that had molted to adults during this period were used for the experiment and were considered to be 1.5 hours old at the end of the interval. At 24 hour intervals, animals were singled and allowed to lay eggs for four hours. The average number of eggs laid per hour per worm was calculated.

Brood size: L4 animals were singled and transferred to new plates daily during the period of egg laying. The total number of progeny produced per worm was determined.

Rate ofpostembryonic development: Eggs were picked to plates and examined one hour later. Animals that had hatched during this period were used for the experiment. The percentage of animals that had reached adulthood by each time point was scored.

Developmental stage of the germline: Worms were synchronized at the final molt as for the time course analyses of egg-laying rate. The proximal portion of the germline was examined using DIC microscopy either immediately afterward (for examining 1.5 h old adults), or 4.5 hours later (for examining 6 hour old adults).

Time course analyses of egg production: Worms were synchronized at the final molt as for the time course analyses of egg-laying rate. Worms were examined at three hour intervals and the percentage of worms containing fertilized eggs in the uterus was determined.

Vulva Formation: Animals were examined under the dissecting microscope and were considered to be Muv if they had more than one vulva.

### 7.2 Suppression of Heterochronic Phenotype

clk-1 mutants have a pleiotropic phenotype that includes an average slowing of embryonic and post-embryonic development, rhythmic behaviors, reproduction and aging (Wong et al., 1995, Genetics 139: 1247-1259). The slow reproduction phenotype was analyzed in detail by counting the number of eggs laid per hour at different time points after the worms had molted into adults (FIG. 1A). Wild-type animals reach their peak egg-laying rate at about 24 hours after molting into adults and have almost finished producing fertilized eggs at 72 hours. The peak of egg laying is delayed in the clk-1(qm30) mutants, as they reach their peak rate at around 72 hours. One possible cause for the delay could be egg retention (the egg-laying defective (Eg1-d) phenotype); however, clk-1 mutants are not Eg1-d. Another possibility for the delay could be excessive spermatogenesis (Hodgkin and Barnes, 1991, Proc R Soc Lond B Biol Sci 246:19-24). C. elegans hermaphrodites first produce sperm, then switch permanently to oocyte production. Thus, neither oocytes nor eggs can be produced before the end of spermatogenesis, and the number of sperm produced determines the brood size. The decreased brood size of clk-1 mutants compared to the wild type suggests that excess spermatogenesis is not the cause of the delayed egg laying (FIG 1B) (Wong et al., 1995, Genetics 139: 1247-1259). A third possibility, which was examined in detail, is that the development of the germline relative to that of the soma is delayed in clk-1 mutants.

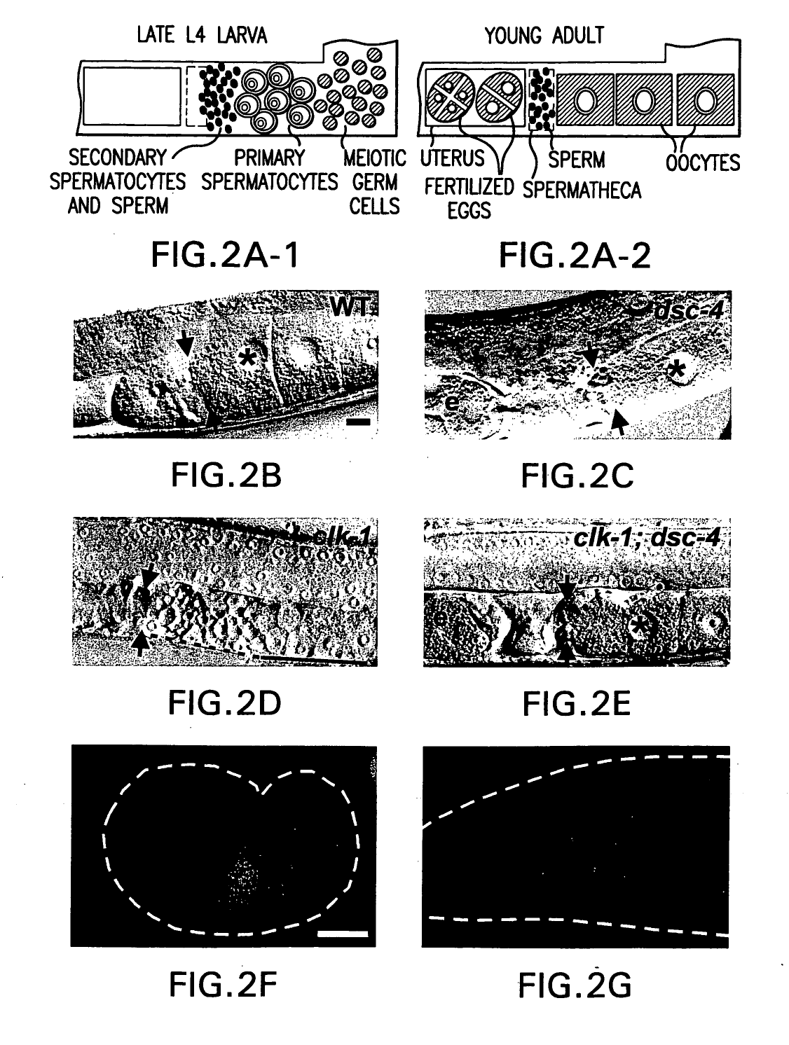

The C. elegans adult hermaphrodite gonad consists of two U-shaped arms (an anterior and a posterior), each of which terminates in a spermatheca (FIG. 2A). The two spermathecae join the gonad arms to the uterus, which stores the fertilized eggs, and fuse at the vulva. When referring to the distal-proximal axis it is relative to the vulva, which is the proximal opening of the gonad to the exterior. The stage of development of the germline is polarized along the distal-proximal axis. Most of spermatogenesis takes place in the proximal gonad. For oogenesis, the distal arm of each gonad forms a syncytium that contains the germ cell nuclei undergoing mitosis. Moving proximally, germ cells exit the mitotic cycle and enter into, and progress through the first stages of meiosis. In the wild-type hermaphrodite, primary spermatocytes, the first gametes to differentiate, are observed at the late L4 stage and oogenesis commences shortly after the hermaphrodites molt into adults. To explore the possibility that the development of the germline is slow in clk-1 mutants, we used DIC microscopy to examine the stage of development of the germline at the proximal end of the anterior and posterior gonads shortly after the worms had molted into adults. At 6 hours after the adult molt, all the wild-type worms examined had oocytes at the proximal end of the anterior and posterior germline, and half the animals also had fertilized eggs (FIG. 1C, 2B). In comparison, the onset of oogenesis is dramatically delayed in clk-1 mutants, in which 97% of the anterior gonads examined in 6 hour old adults were still undergoing spermatogenesis, and only 3% had initiated oogenesis (FIG 1C, 2D). Interestingly, although the development of the germline of the posterior gonad of clk-1 mutants is also delayed compared to that of the wild type, it is remarkably less affected than the anterior gonad: 28% have begun oogenesis and 19% already have fertilized eggs (FIG 1C).

To determine whether the onset of gamete differentiation (that is, spermatogenesis) was also delayed in clk-1 mutants, the proximal anterior arm of the germline was examined just after the adult molt. When wild-type worms were examined 1.5 hours after they had molted into adults, the majority (87%) had completed primary spermatocyte formation (FIG 1D). In contrast, the majority of clk-1 mutants were either before (42%) or in the process (48%) of primary spermatocyte formation. Together, these results indicate that clk-1 mutants show a heterochronic phenotype: the overall development of the germline of the mutants at a given stage of somatic development is delayed relative to wild-type animals, with the stage of the germline of clk-1 mutants at the adult molt corresponding to that of wild-type animals at the mid- to late-L4 stage. dsc-4(qm182) was isolated as a suppressor of the slow defecation phenotype of clk-1 mutants (Branicky et al., 2001, Genetics 159:997-1006). Although the dsc-4 mutation does not suppress all aspects of the clk-1 phenotype, closer examination has revealed that it does in fact suppress several other phenotypes. As described above, wild-type worms reach their peak of egg laying between 24 and 48 hours after the molt to adulthood, whereas clk-1 mutants only reach their peak of egg laying at 72 hours. The dsc-4 mutation suppressed this delay as the peak egg-laying rate of the clk-1/dsc-4 double mutants was reached by about 48 hours (FIG 1A). Given that the delayed egg laying of clk-1 mutants is due to their delayed production of oocytes (see above), these results suggested that the dsc-4 mutation suppressed the delayed oogenesis of clk-1 mutants.

The effect of the dsc-4 mutation on the development of the germline was examined directly through observation of the germline of clk-1/dsc-4 double mutants using DIC microscopy. At 6 hours after the adult molt, 50% of the clk-1/dsc-4 double mutants had oocytes at the proximal end of the anterior germline (41% without eggs in the uterus, 9% with eggs; FIG 1C, 2E) compared to only 3% of the clk-1 single mutants. The proximal end of the posterior germline was even more advanced by dsc-4(qm182) than the anterior, as 100% of the double mutants were undergoing oogenesis (97% with eggs in the uterus). This suggested that, although dsc-4 can dramatically suppress the overall slow germline development of clk-1 mutants, there is an additional defect in the anterior gonad of clk-1 mutants that cannot be overcome. The proximal germline was examined at earlier time points and revealed that the dsc-4 mutation also suppresses the delayed onset of gametogenesis. At 1.5 hours after the adult molt, 100% of the germline examined in the clk-1/dsc-4 double mutants had finished primary spermatocyte production, compared to only 10% of the clk-1 single mutants (FIG 1D). This corresponded to a full suppression of the heterochronic defect observed in the clk-1 mutants as the clk-1/dsc-4 double mutants began spermatogenesis and oogenesis at the correct stages relative to those of the development of the soma.

The effect of the dsc-4(qm 182) mutation on the rate of postembryonic development was analyzed to determine whether the suppression by dsc-4 was caused by an acceleration of germline development rather than by retardation of somatic development. Wild-type worms reached adulthood between 45 and 51 hours after hatching (FIG. 1E). The dsc-4 mutants have slower post-embryonic development than the wild type as they only reached adulthood between 57 to 69 hours after hatching. However, the duration of post-embryonic development of the clk-1/dsc-4 double mutant is almost identical to that of the clk-1 mutants. Both clk-1 and clk-1/dsc-4 mutants reached adulthood between 63 and 81 hours after hatching. This indicated that the dsc-4 mutation suppressed the heterochronic defect of clk-1 mutants by re-synchronizing the development of the germline with that of the soma without slowing down somatic development.

The mutant was rescued with the cosmid K02D7 and with a PCR product that contained the predicted sequence K02D7.4. The PCR product rescued the fast defecation of clk-1/dsc-4 double mutants as well as the fast egg-laying rate. Expression of K02D7.4 was inhibited with RNAi in clk-1/dsc-4 and wild-type backgrounds. K02D7.4 RNAi was found to phenocopy the dsc-4(qm182) mutation in the clk-1 background. The defecation rate as well as the egg-laying rate of clk-1(qm30)/K02D7.4 RNAi animals was similar to that of the clk-1(qm30)/dsc-4(qm182)double mutants (data not shown). However, the effect of K02D7.4 RNAi was not additive to that of the dsc-4 mutation for defecation or egg laying in the clk-1 background. Also, K02D7.4 RNAi in dsc-4 mutants did not cause any obvious enhancement of the phenotype. Together, these results demonstrated that the gene in the genomic cosmid clone K02D7.4 is dsc-4, and that the dsc-4(qm182) allele is probably null. dsc-4 encodes an 892-residue protein, which is similar in sequence to the large subunit of the microsomal triglyceride transfer protein (MTP; FIGS. 3A, C and 4). MTP is an endoplasmic reticulum (ER) protein that is necessary of the secretion of apolipoprotein B (apoB)-containing lipoproteins, in particular LDLs (Berriot-Varoqueaux et al., 2000, Ann, Rev. Nutr. 20:663). Lipoproteins consist of a high-molecular weight protein complexed to various lipids, including triglycerides, cholesteryl esters, cholesterol, and phospholipids.

In humans, mutations in the large subunit of MTP cause abetalipoproteinemia (ABL), a severed deficiency in LDL secretion (Nakamuta et al., 1996, Genomics 33:313-316 and Sharp et al., 1993, Nature 365:65-69). The DSC-4 polypeptide has a putative N-terminal signal sequence for secretion, with a predicted cleavage site between residues 18 and 19 (FIG. 4). DSC-4 has a single clear homologue in every animal species whose genome has been sequenced, but appears to be absent from plants and unicellular organisms. The identities between homologues extend across the entire sequence and are not confined to particular regions or domains, though domains of greater similarity exist, and the alignment of DSC-4 with vertebrate MTPs does not require the introduction of numerous gaps (FIG. 3A). When DSC-4 was compared to the NCBI non-redundant protein database using PSI-blast, the 7 proteins with the highest scores were bona fide vertebrate MTPs, including human, rat, mouse, bovine, pig, and zebrafish MTPs, as well as anopheles and drosophila MTPs. The dsc-4(qm 182) allele was found to carry two point mutations resulting in amino acid substitutions, one of which is highly conserved between species (FIG. 3A). MTP has an apoB binding domain, an apoB and PDI binding domain, as well as a lipid binding and transfer domain (FIGS. 3C and 4) (Mann et al.,1999, J Mol Biol 285:391-408). The mutation sites of dsc-4(qm182) were in the apoB-binding domain and were different from those found in abetalipoproteinemia patients.

dsc-4 expression was analyzed using dsc-4::GFP fusion proteins (transcriptional or translational reporters). The two types of reporters showed almost identical expression patterns. The fusion protein derived from the translational reporter encoded a full length dsc-4 sequence and was capable of rescuing the mutant phenotype, suggesting that the pattern of expression observed reflected that of endogenous dsc-4. However, the GFP fluorescence filled the cytoplasm rather than being confined to the endoplasmic reticulum (ER). Possibly, overexpression of this protein in the ER was difficult and abnormal localization was due to overexpression. GFP fluorescence was observed in the intestine from early embryogenesis, just after the beginning of elongation (FIG. 2F) throughout larval stages (FIG. 2G) and adulthood, and was also observed in the intestine of males. In the worm, the very large intestine is the digestive organ and the major secretory organ. In particular, the intestine secretes the vitellogenins, which are apoB homologues. Thus, the expression of dsc-4 was consistent with the pattern of expression of MTP in vertebrates, which is found predominantly in the intestine and the liver.

### 8. EXAMPLES

### Validation of dsc-4

dsc-4 rescues a clk-1 mutant C. elegans by reducing secreted LDL-like lipoproteins (as evidenced by its homology to MTP). LDL-like lipoprotein levels were manipulated in a clk-1 single mutant C. elegans in other ways (e.g., by cholesterol depletion, vit RNAi, or SOD RNAi) to examine if they could phenocopy the clk-1/dsc-4 double mutant.

### 8.1 Materials and Methods

8.1.1 Strains and Culture Methods

Animals were cultured at 20° C. as described (Brenner, 1974, Genetics 77:71-94) and were fed the E. coli strain OP50 unless otherwise indicated. Cholesterol depletion experiments were performed on standard NGM plates seeded with OP50, but without any added cholesterol (NGM-C). Worms were cultured on the NGM-C plates for two or more generations before the rate of germline development was examined. For RNAi experiments, worms were cultured on NGM plates supplemented with 1 mM IPTG and 50 μg/mL ampicilin, and were fed the E. coli strain HT115 transformed with pPD129.36 derived plasmids. The F1 generation was examined.

The wild-type strain was N2 (Bristol strain). The following mutations were used: clk-1(qm30) III; unc-33(e204), dpy-9(e12), dsc-4(qm182) IV.

8.1.2 RNA Interference

For SOD RNAi clones, PCR products were amplified from a first-strand cDNA library (generated by the reverse transcription of total RNA isolated from mixed-stage N2 worms using random primers). The PCR products were cloned into the SmaI site of pPD129.36. The following regions were used: for SOD-1, 17287-18262 of C15F1; for SOD-2, 1216-2354 of F10D11; for SOD-3, 14358-15759 of C08A9; for SOD-4, 1253-2977 of F55H2.

For vit RNAi clones, PCR products were amplified from N2 genomic DNA, digested and cloned into pPD129.36. For vit-2, an internal EcoRI-SaII fragment was cloned (24381-25252 of C42D8). For vit-5, a PCR product corresponding to 9271-10130 of C04F6 was cloned into the PstI and XbaI sites. For vit-6, a PCR product corresponding to 5787-6851 of K07H8 was cloned into the XhoI and HindIII sites. All inserts were sequenced to confirm the identity of the vit gene cloned.

RNAi experiments were performed as described (Kamath et al., 2001, Genome Biol. 2: research0002.1-research0002.10). The controls were fed the HT115 bacteria transformed with the pPD129.36 vector.

8.1.3 Phenotypic Analyses

Time course analysis of egg-laying rate: L4-stage animals were picked to plates and examined three hours later. Animals that had molted to adults during this period were used for the experiment and were considered to be 1.5 hours old at the end of the interval. At 24 hour intervals, animals were singled and allowed to lay eggs for four hours. The average number of eggs laid per hour per worm was calculated.

Brood size: L4 animals were singled and transferred to new plates daily during the period of egg laying. The total number of progeny produced per worm was determined.

Rate ofpostembryonic development: Eggs were picked to plates and examined one hour later. Animals that had hatched during this period were used for the experiment. The percentage of animals that had reached adulthood by each time point was scored.

Developmental stage of the germline: Worms were synchronized at the final molt as for the time course analyses of egg-laying rate. The proximal portion of the germline was examined using DIC microscopy either immediately afterward (for examining 1.5 h old adults), or 4.5 hours later (for examining 6 hour old adults).

Time course analyses of egg production: Worms were synchronized at the final molt as for the time course analyses of egg-laying rate. Worms were examined at three hour intervals and the percentage of worms containing fertilized eggs in the uterus was determined.

### 8.2 VIT Knockdown/Mutation

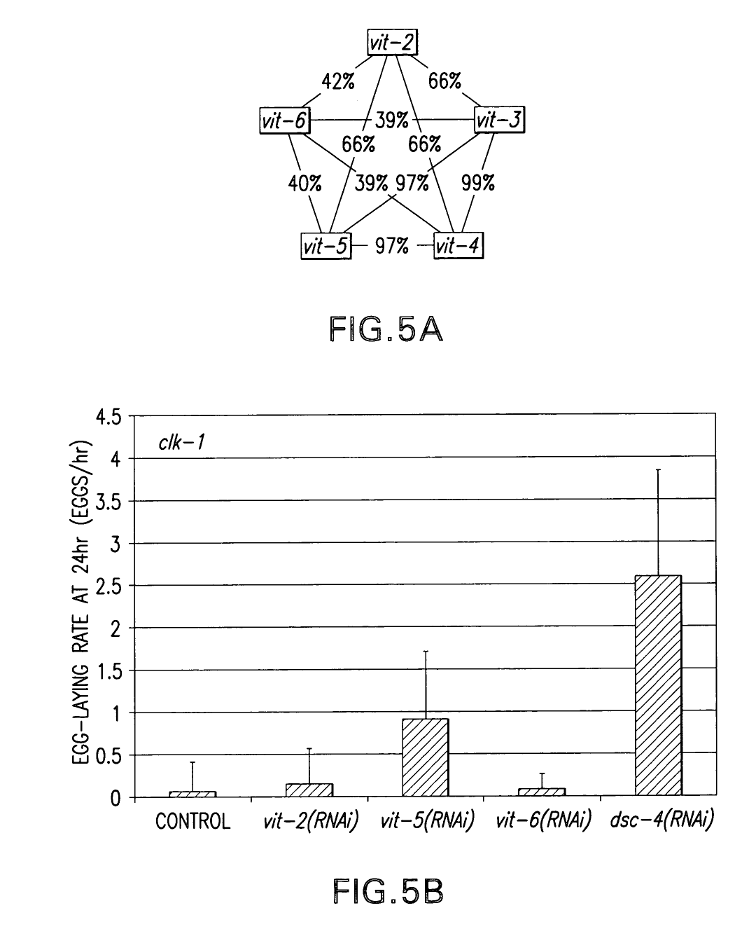

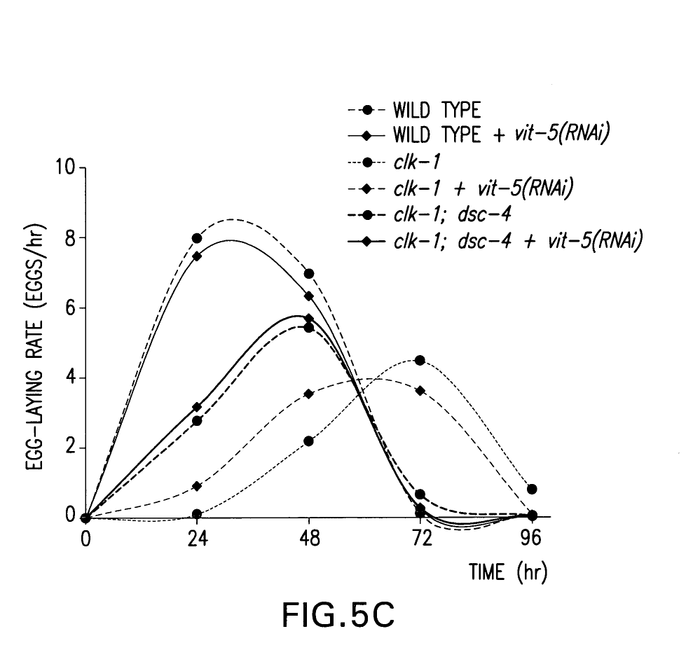

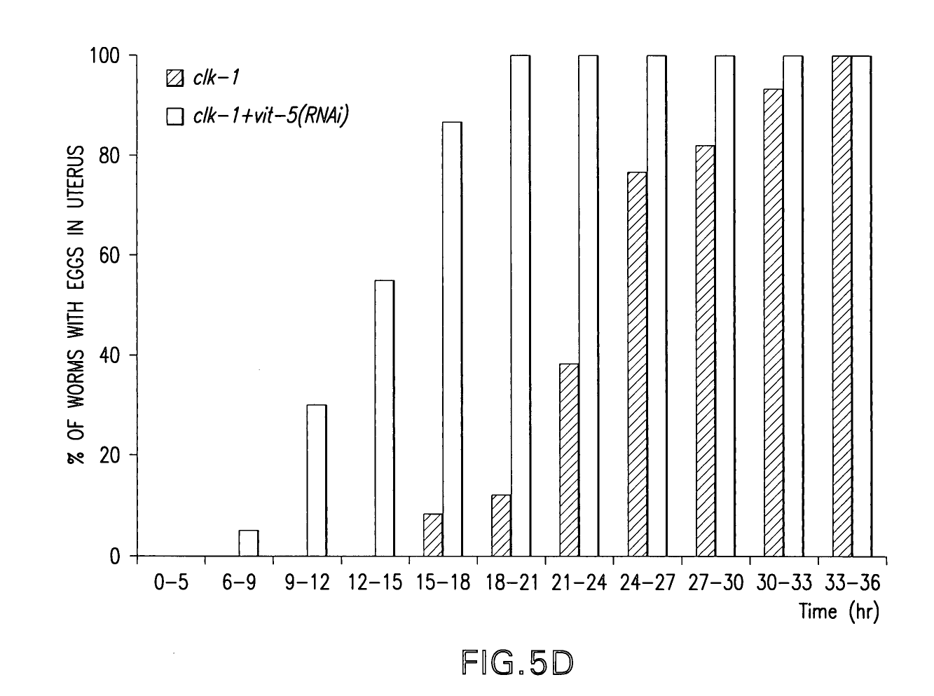

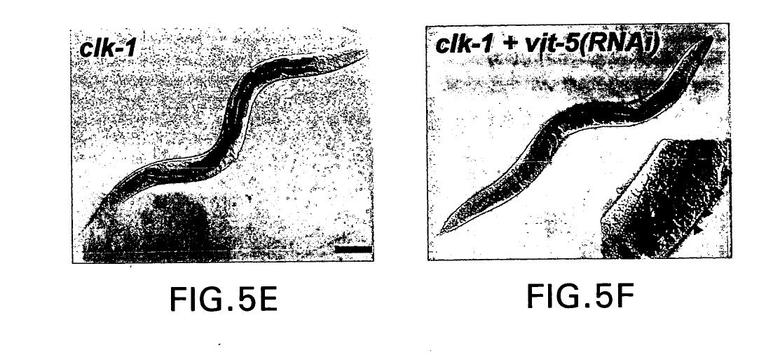

In mammals, MTP is required for the secretion of apoB-containing lipoproteins. The genome of C. elegans contains five apoB-like genes (vit-2, -3, -4, -5 and -6) and one apoB-like pseudogene (vit-1) (Blumenthal et al., 1984, J Mol Biol 174:1-18; Spieth and Blumenthal, 1985, Mol Cell Biol 5:2495-2501; Spieth et al., 1985, Nucleic Acids Res 13:7129-7138). The vit genes (vit-2, -5 and -6) were disrupted by RNAi to determine whether altered secretion of the C. elegans apoB-like genes could phenocopy the effect of dsc-4 on germline development. Since the coding region of the vit-5 RNAi clone is 98% identical to both vit-3 and -4, the vit-5 RNAi treatment is also expected to disrupt the function of vit-3 and -4 (FIG. 5A).

The effect of vit RNAi on the egg-laying rate of clk-1 mutants was analyzed. The most significant effect was observed 24 hours after the animals had molted into adults (FIG. 5C). At that time, the clk-1/vit-5 RNAi animals had begun to lay eggs while the clk-1 controls had not (FIG. 5B,C). The peak of egg laying rate of the clk-1/vit-5 RNAi animals was reached approximately 12 hours prior to that of the clk-1 controls (FIG. 5C). The effect of vit-5 RNAi was not additive to the effect of dsc-4, as expected if the effect of the dsc-4 mutation was due to reduced VIT protein secretion. The effect of RNAi against the other vit genes was extremely weak by that measure (FIG. 5B). No effect on defecation was seen for any of the genes.

A time course experiment was done to examine the rate of germline development in vit-5 RNAi animals to determine whether the increased rate of egg laying produced was caused by an increase in the rate of germline development. The percentage of animals that contained fertilized eggs in their uteri was determined for clk-1 single mutants and clk-1/vit-5 RNAi double mutants. clk-1 mutant animals started having fertilized eggs between 15 to 18 hours after they have molted to adults, and it takes 36 hours for all worms to contain fertilized eggs. In contrast, clk-1/vit-5 RNAi double mutants started having fertilized eggs between 6 to 9 hours and all contain fertilized eggs by 21 hours (FIG. 5D). As vit-5 RNAi appeared to have the most significant effect on egg laying at 24 hours (FIG. 5C), the average number of eggs in uteri at that time was scored. At that time, clk-1/vit-5 RNAi double mutants contained more than 10 times more eggs in their uteri than the clk-1 animals (FIGS. 5E and F). Thus, knocking down vit-5 expression suppressed the slow germline development of clk-1 mutants.

The time course analysis indicated that scoring the presence of eggs in the uterus is a very sensitive measure of the effect of the vit-5 RNAi. vit-2 RNAi and vit-6 RNAi were also scored in this way at 18 hours after the adult molt, which succeeded in revealing a small effect for these genes (percent animals with eggs in the uterus: clk-1=5.2%, clk-1/vit-2 RNAi=30.4%, clk-1/vit-6 RNAi=25.0%, clk-1/vit-5 RNAi=87%; n>44).

In summary, reduced production of the apoB homologues vit-3, -4 and-5, and, to a much lesser degree, vit-2 and -6, appeared to mediate the effect of dsc-4 on germline development. One possibility for why the effects observed with the vits were less severe than those observed with dsc-4 (including no effect on defecation) is that RNAi against the vit genes was not efficient because of their high level of expression (Kimble and Sharrock, 1983 Dev Biol 96:189-196; Sharrock, 1983, Dev Biol 96:182-188). Another possibility is that all the vit genes would have to be targeted at once for maximum effect. This possibility is also supported by the observation that, although the vits function as essential yolk proteins and RNAi has no significant effect on the ability to produce eggs (FIG. 5).

The vit genes were originally isolated as genes that encode yolk proteins. vit-2 and -6 are mainly synthesized in the intestine of hermaphrodites and are transported to the germline from the intestine (Kimble and Sharrock, 1983, Dev Biol 96:189-196). It has been reported that vit-5 shows a strong hermaphrodite-specific signal by northern blotting (Blumenthal et al., 1984, J Mol Biol 174:1-18) which suggests that vit-5 and/or vit-3, -4 (which are 97% identical to vit-5) also encode yolk proteins. However, our results indicated that the vits also function in lipoprotein particles that resemble the apoB-dependent LDL particles found in vertebrates and that might be distinct from the yolk lipoproteins. Two pieces of evidence suggest this. First, disruption of dsc-4 does not affect brood-size. If dsc-4 was required for yolk production, mutation in dsc-4 should drastically reduce brood size like mutations in rme-2, which encodes a receptor for yolk proteins (Grant and Hirsh, 1999, Mol Biol Cell 10:4311-4326). Second, yolk protein production should only be necessary in hermaphrodites, but dsc-4 is also expressed in the intestine of males.

### 8.3 Cholesterol Depletion

Because dsc-4 caused a decrease in secreted LDL-like lipoproteins, experiments were conducted to determine if dsc-4 mutations could be phenocopied by depletion of cholesterol. In mammals, cholesterol is a major constituent of lipoproteins, and reducing its intake or synthesis leads to reduced levels of LDL. Since C. elegans cannot synthesize cholesterol, decreasing the amount of dietary cholesterol causes a decrease in internal cholesterol levels (Crowder et al., 2001, J Biol Chem 276:44369-44372). The effect of cholesterol depletion on germline development was examined in wild-type, clk-1, dsc-4, and clk-1/dsc-4 backgrounds (FIG 1D). Cholesterol depletion completely abolished the slow germline development of clk-1 mutants, but had only a mild effect on other genotypes. The effect of low cholesterol on the clk-1 germline was indistinguishable from that of dsc-4 in the presence of cholesterol, suggesting that, as in mammals, the reduction in dietary cholesterol reduces the secretion of the LDL-like particles.

Although cholesterol depletion mimicked the effect of the dsc-4 mutation on germline development, in general, the effect of cholesterol depletion was much more severe and included numerous defects not seen in dsc-4 mutants (Crowder et al., 2001, J Biol Chem 276:44369-44372; Gerisch et al., 2001, Dev Cell 1:841-851; Merris et al., 2003, J Lipid Res 44:172-181; Shim et al., 2002, Mol Reprod Dev 61:358-366; Yokoyama, 2000, Ann N Y Acad Sci 902:241-247). This difference could be explained in various ways. One possibility is that dsc-4 polypeptide is not as stringently required for secretion of LDL-like lipoproteins in worms as is MTP in mammals. Another possibility is that there are other pathways of cholesterol redistribution from the intestine to peripheral tissues in worms, which makes the disruption of one pathway much less severe than an overall deficit in cholesterol intake. In mammals, for example, there are forms of lipoprotein, such as HDL, that do not require MTP for secretion. Finally, some of the defects observed in cholesterol-depleted worms might be the result of various processes requiring cholesterol in the intestine itself, which would not manifest themselves when only redistribution from the intestine is affected.

### 8.4 SOD-1 Knockdown/Mutation

clk-1 encodes a putative hydroxylase, which is required for the biosynthesis of UQ (Stenmark et al., 2001, J Biol Chem 276:33297-33300). In clk-1 mutants, UQ is absent and the biosynthetic intermediate, demethoxyubiquinone (DMQ), accumulates instead (Miyadera et al., 2001, J Biol Chem 276: 7713-7716). One of the important roles of UQ is as an electron carrier in the mitochondrial respiratory chain. In this role, UQ is one of the main sites of reactive oxygen species (ROS) production (Raha and Robinson, 2000, Trends Biochem Sci 25:502-508). However, UQ also has several other cellular roles including, somewhat paradoxically, as an antioxidant. The redox properties of DMQ are quantitatively different from those of UQ, in particular, DMQ might be less prone to ROS production (Miyadera et al., 2002, FEBS Lett 512:33-37). Together with the observation that treatment with an antioxidant, vitamin E, slows down germline development in C. elegans (Harrington and Harley, 1988, Mechanisms of Aging and Development 43:71-78.), this raises the possibility that the effect of clk-1 on the development of the germline may be mediated through an alteration of the metabolism of ROS. If the developmental timing of the germline is redox-regulated and the redox properties of DMQ result in less oxidative stress than UQ, reducing the function of antioxidant enzymes such as the superoxide dismutases (SOD) could suppress the abnormal timing of germline development by increasing the amount of ROS.

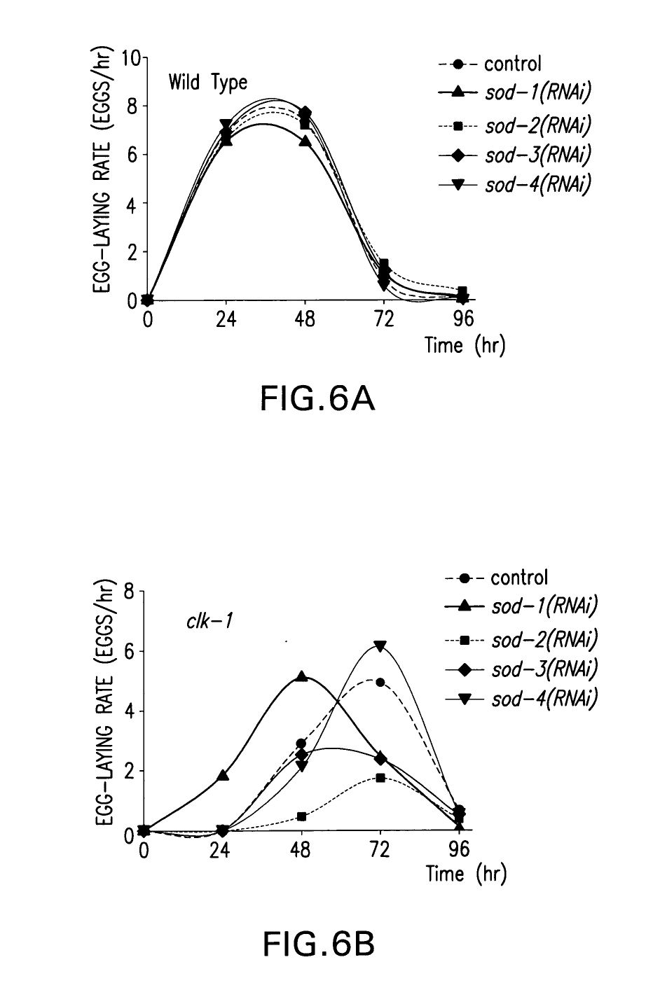

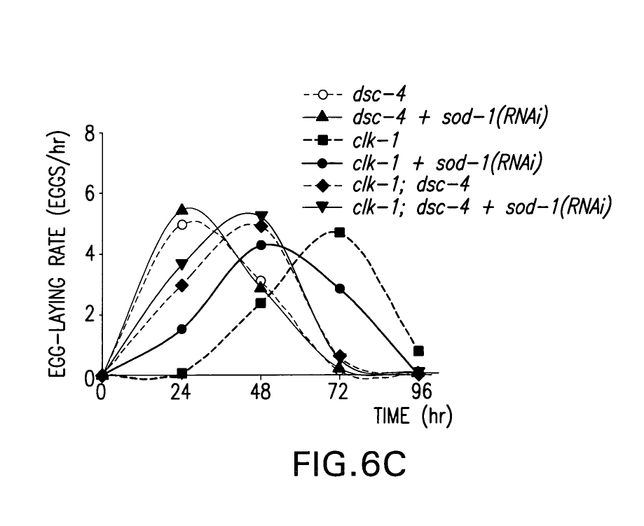

The effect of reducing SOD activity by RNAi was examined to test whether ROS levels affect the developmental rate of the germline. When levels of SOD-1,- 2, -3, and -4 were decreased by RNAi, no effect was seen on the egg-laying rate of wild-type animals (FIG. 6A). However, in a clk-1 mutant, SOD-1 RNAi suppressed the delayed egg production (FIG. 6B). This effect was not seen when RNAi against any of the other genes was administered. clk-1 mutants reached their peak of egg laying at 72 hours, whereas clk-1/SOD-1 RNAi double mutants reached their peak of egg laying by 48 hours. These results suggest that the slow development of the germline in clk-1 mutants can be relieved by increasing the amount of superoxide generated in the cytoplasm (since SOD-1 is the cytoplasmic Cu/Zn superoxide dismutase). Since the central defect in clk-1 mutants is the accumulation of DMQ instead of UQ, this suggests that DMQ leads to lower levels of superoxide than when UQ is present.

Although SOD-1 RNAi suppressed the slow germline development of clk-1 mutants, it did not have any effect on the clk-1/dsc-4 double mutants (FIG. 6C). This suggests that sod-1 and dsc-4 function in the same process or signaling pathway to suppress the slow germline development of clk-1 mutants. As shown above, the effect of dsc-4 mutation on the germline development of clk-1 mutants is very likely through its role in affecting the secretion of LDL-like lipoprotein. The observation that sod-I RNAi had the same effect as a dsc-4 mutation, together with previous findings that lipid oxidation is reduced in clk-1 mutants (Braeckman et al., 2002, Mech Aging Devel 123:1447-56) and that the cytoplasmic Cu/Zn-SOD regulates the level of LDL oxidation in other systems (Guo et al., 2001, Arterioscler Thromb Vasc Biol 21:1131- 1138) strongly suggests that the oxidation of LDL-like lipoproteins is an important factor in controlling the development of the germline in C. elegans.

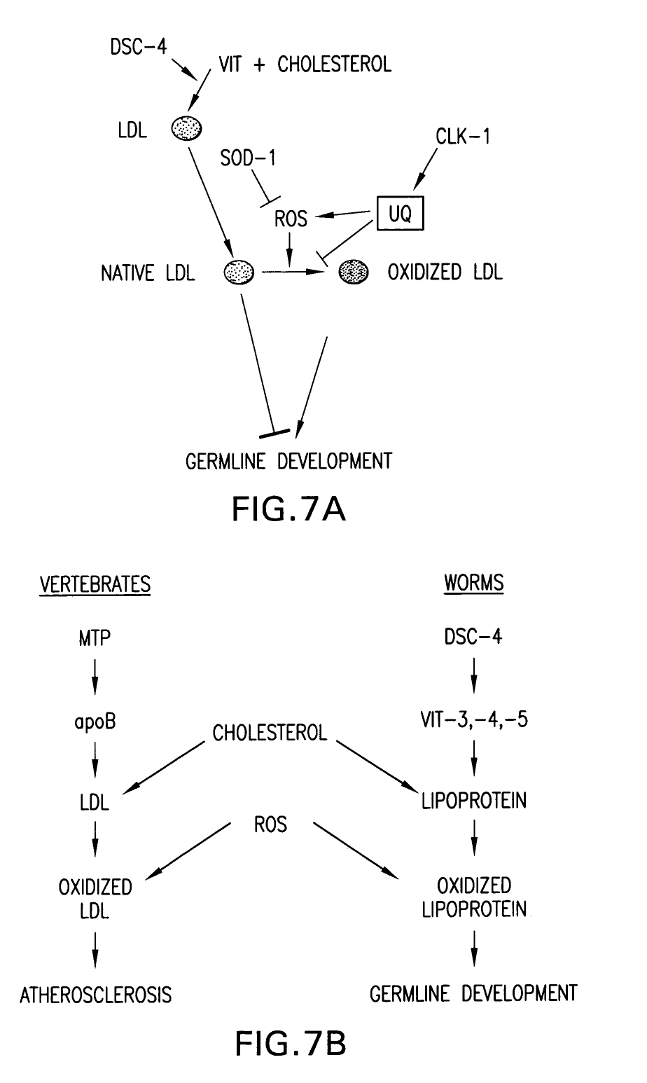

In conclusion, it appears that decreasing the oxidation of LDL-like lipoproteins slows down the rate of germline development relative to that of the soma (as in clk-1 mutants), but that the proper coordination of soma and germline can be restored, either by interfering with the function of SOD-1, or by decreasing the production and secretion of native LDL-like lipoproteins (as in clk-1/dsc-4 double mutants). Thus, oxidized lipoprotein stimulate, and native lipoprotein inhibit germline development (FIG. 7A).

### 9. EXAMPLE

### Dsc-3

### 9.1 Identification and Characterization

The dsc-3 gene was mapped to a genetic region of 0.5 cM on chromosome 4 of C. elegans. However, the dsc-3 gene could not be cloned by the standard transformation rescue technique because injection of DNA from cosmids corresponding to this genetic interval failed to rescue the dsc-3 mutants.

A candidate approach was taken to identify dsc-3. Predicted genes in the genomic region where dsc-3 mutations had been mapped were investigated and one potential candidate, the predicted nucleotide sequence H06H21.10 was identified from a sequence database available from the internet web site wormbase.org. The predicted nucleotide sequence H06H21.10 was annotated as having similarity to the DNA encoding a human gene. Because the predicted H06H21.10 gene was very large (>13 kb) and was not fully contained on any available cosmid, rescue experiments could not easily be performed.

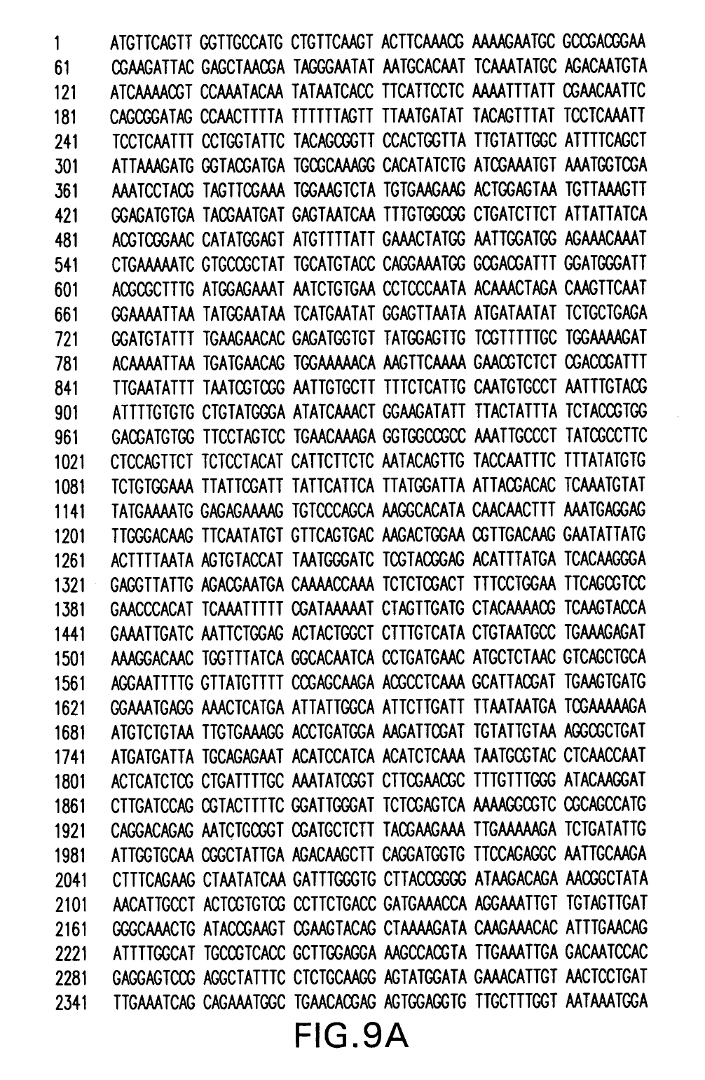

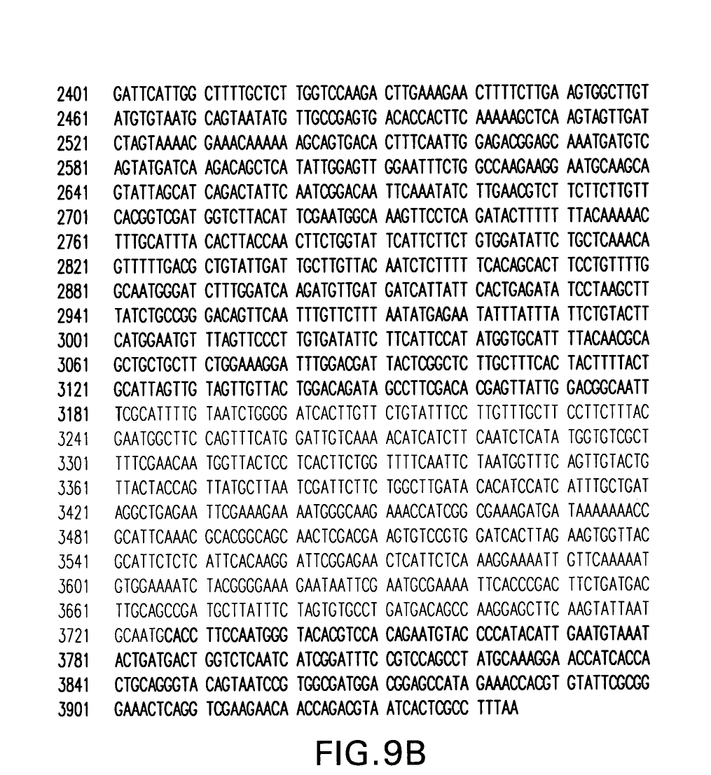

To further examine the predicted H06H21.10 gene, the genomic sequence was compared to the nucleotide sequence of the human gene. Further analysis and refinement of the alignment of the sequences revealed that two additional exons are present in the region but that-were not identified in the worm sequence database as coding sequences of the predicted H06H21.10 gene. According to the invention, the correct coding sequence of this gene, referred to herein as dsc-3, comprises 3945 bp and encodes for a protein with 1314 amino acids, see FIG. 9 and SEQ ID NO:7. The dsc-3 coding sequence comprises an additional 276 bps (nucleotides 459-734) which correspond to the addition of one exon between exon 4 and exon 5 of the predicted H06H21.10 gene (nucleotides 459-613) and a second addition 121 nucleotides 5′ to the predicted exon 5 (nucleotides 614-734). The predicted sequence of the H06H21.10 gene reported in the database is incorrect. According to the invention the 22 exons of dsc-3 were identified at the following nucleotide base pairs: exon 1: 1-110, exon 2: 111-224, exon 3: 225-323, exon 4: 324-458, exon 5: 459-613, 614-991, exon 7: 992-1272, exon 8: 1273-1341, exon 9: 1342-1764, exon 10: 1765-1890, exon 11: 1891-2088, exon 12: 2089-2232, exon 13: 2233-2320, exon 14: 2321-2469, exon 15: 2470-2620, exon 16: 2621-3014, exon 17: 3015-3147, exon 18: 3148-3476, exon 19: 3477-3693, exon 20: 3694-3791, exon 21: 3792-3909, and exon 22: 3910-3945 of SEQ ID NO:7.

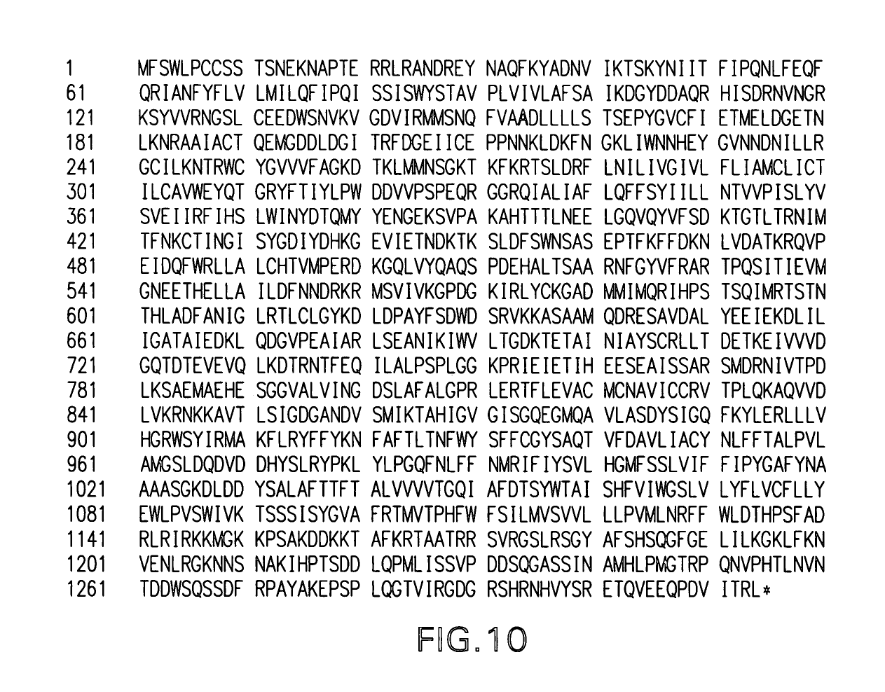

The predicted amino acid sequence encoded by dsc-3 is shown in FIG. 10 (SEQ ID NO:8), hereafter referred to as DSC-3. An alignment of the amino acid sequence of DSC-3 and four homologous Type IV P-Type ATPases from humans (SEQ ID NOs:9, 10, 11, and 12) is shown at FIG. 11. ATP8B 1 shares highest amino acid identity with ATP8B2 and ATP8B4. The degree of amino acid identity between the DSC-3 and the ATP8B4 sequence is 54% when the amino acids are aligned from DSC-3: 137 to 1115 and ATP8B4: 2 to 946.

### 9.2 RNA Interference

RNA interference (RNAi) was used to reduce the expression of dsc-3. Exons 18 and 19 were amplified by PCR from a first-strand cDNA library (generated by the reverse transcription of total RNA isolated from mixed-stage N2 worms using random primers). The PCR product (corresponding to nucleotides 3182-3666 of the dsc-3 transcript) was cloned into the PstI and NheI sites of pPD129.36, which are flanked by the T7 promoter. Primers complementary to the T7 promoter were used to amplify the dsc-3 fragment from the clone. This PCR product was used as a template for an in vitro transcription reaction to produce double-stranded RNA (dsRNA). This dsRNA was injected into clk-1(qm30) mutants at a concentration of ˜μg/mL as described in Fire et al., 1998 (Nature. 391(6669):806-11.) Worms exhibiting reduced expression of dsc-3 were able to fully phenocopy (produce the same phenotype as) the dsc-3 mutants. It is clear from the results of this RNAi experiment that the predicted dsc-3 is a dsc gene, as defined above in Section 9.1, as reducing its function produces a Dsc phenotype.

### 9.3 DSC-3 and Lipid Metabolism

Three dsc-3 mutants, dsc-3(qm179), dsc-3(qm180), and dsc-3(qm184) were isolated as suppressors of the slow defecation phenotype of clk-1 mutants. The dsc-3 mutants have the same phenotype as the dsc-4 mutants, that is, they suppress the slow defecation of clk-1 mutants at 20° C. as well as after shifts to 25° C. The dsc-3 mutants also show the accelerated germline development observed in dsc-4 mutants. In addition, the effect of the dsc-3(qm179) mutation is not additive to that of the dsc-4(qm 182) mutation, which suggests that these two mutations act in the same pathway or affect the same process. dsc-3 encodes a Type IV P-type ATPase, more specifically, an ATP-dependant amino-phospholipid transporter dsc-3 is 50% identical to the amino acid sequence encoded by human gene ATP8B1, which corresponds to the human disease locus for FIC1/BRIC/PFIC1. ATPase enzymes are a large family of enzymes that are integral membrane proteins which transport agents such as metals, ions, and phospholipids across a membrane using ATP (Harris et al, 2003, Biochim. Biophys. 1633:127-131). Such enzymes generates an asymmetry of lipid composition between the two leaflets of a membrane and confers particular properties to such a membrane (Daleke, 2003, Journal of Lipid Research 44:233-42). Mutations in one such ATPase, the human ATP8B1/FIC1, results in a cholestatic phenotype characteristic of Byler's disease (Bull et al., 1998, Nature Genetics 18:219-24; Trauner et al, 2002, Physiol. Rev. 83:633-671). ATPase enzymes such as, but not limited to, ATP8B1/FIC1 play a role in bile acid transport and secretion. ATP8B1/FIC1 has been expressed in CHOK1 cells and then identified in membranes of those cells where an altered the distribution of lipids in the membrane was observed (Ujhazy et al., 2001, Hepatology 34:768-775).

Cholestatic diseases are conditions in which bile flow within the liver is impaired. In mammals, cholesterol homeostasis is achieved through the coordinate regulation of its intestinal absorption, endogenous synthesis, and biliary excretion. Altering bile metabolism is one of the methods for controlling cholesterol and LDL levels (Lu et al., 2001, Trends in Endocrinology and Metabolism 12:314-20; Fuchs, 2003, Am J Physiol Gastrointest Liver Physiol. 284:G551-7). Although the relationship between defective aminophospholipid transport and cholestasis is not precisely understood, ATP8B1 plays an important role in bile salt excretion (Bull et al., 1998, Nature Genetics 18:219-24), for example, regulation of the activity of bile acid transporters by maintaining lipid asymmetries in the membrane of the liver canaliculi. In addition, because this gene is expressed in the liver but more predominantly in the intestine, lipid asymmetries across membranes can also affect the activity of membrane transporters involved in sterol absorption and secretion in the intestine (Ujhazy et al., 2001, Hepatology 34:768-75).

Given that dsc-4 encodes an activity necessary for LDL-like lipoprotein secretion, and that lowering LDL-like lipoprotein secretion or lowering cholesterol has the same effect on the rate of germline development as dsc-3, the inventors conclude that dsc-3 indeed affects cholesterol homeostasis in worms as its homologs do in humans. The sequence analysis of dsc-3 supports the invention of using dsc-3 (and its homologs, including human homologs) as a target to screen for compounds for the treatment and/or prevention of atherosclerosis, liver and intestinal problems related to cholesterol metabolism in humans.

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed is:

1. A method for identifying a compound that modulates the level of a lipid or lipoprotein in a nematode comprising:
(a) contacting a compound with test nematodes;
(b) comparing a phenotype of said test nematodes with said phenotype of nematodes not contacted with said compound, wherein a modification of said phenotype correlates with a modulated level of a lipid or lipoprotein, said phenotype being selected from the group consisting of (i) length of defecation cycle; (ii) rate of germ line development relative to rate of soma development; (iii) rate of embryonic development; and (iv) rate of post-embryonic development,
whereby a difference in said phenotypes identifies said compound.

2. A method for isolating a gene that modulates the level of a lipid or lipoprotein in nematodes comprising:
(a) subjecting nematodes that comprise at least one mutation in the clk-1 gene to mutagenesis to produce test nematodes;
(b) identifying test nematodes that exhibit a phenotype that is modified as compared to the phenotype of nematodes not subjected to mutagenesis, wherein said phenotype that is modified indicates a modulated level of a lipid or lipoprotein, said phenotype being selected from the group consisting of (i) length of defecation cycle; (ii) rate of germline development relative to rate of soma development; (iii) rate of embryonic development; and (iv) rate of post-embryonic development; and
(c) cloning the gene that was mutated in step (a) from a test nematode of step (b).

3. A method for identifying a gene that modulates the level of a lipid or lipoprotein in nematodes comprising (a) contacting test nematodes that comprise at least one mutation in the clk-1 gene with a nucleic acid that reduces specifically the level of expression of a nematode gene; and (b) identifying test nematodes that exhibit a phenotype that is modified as compared to the phenotype of nematodes not contacted with said nucleic acid, wherein a modification of said phenotype in said test nematodes indicates that the nematode gene in said test nematodes modulates the level of a lipid or lipoprotein, said phenotype being selected from the group consisting of (i) length of defecation cycle; (ii) rate of germline development relative to rate of soma development; (iii) rate of embryonic development; and (iv) rate of post-embryonic development.

4. The method of claim 3, wherein the nucleic acid is selected from the group consisting of an antisense nucleic acid, and a double-stranded RNA molecule.

5. The method of claim 4, wherein said contacting comprises feeding nematode with bacteria comprising said nucleic acid.

6. A method for selecting nematodes having modulated level of a lipid or lipoprotein comprising:
(a) treating test nematodes to modulate the level of a lipid or lipoprotein; and
(b) identifying test nematodes that exhibit a phenotype that is modified as compared to the phenotype of nematodes that has not been treated, wherein a modulated level of a lipid or lipoprotein in said test nematodes is indicated by the phenotype that is modified, said phenotype selected from the group consisting of (i) length of defecation cycle; (ii) rate of germline development relative to rate of soma development; (iii) rate of embryonic development; and (iv) rate of post-embryonic development.

7. The method of claim 1 or 6, wherein the test nematodes comprise (a) at least one mutation in the clk-1 gene; (b) at least one mutation in the clk-1 gene and at least one mutation in the dsc-3 gene; or (c) at least one mutation in the clk-1 gene and at least one mutation in the dsc-4 gene.

8. The method of claim 1, 2, 3, 4, 5, or 6, wherein the phenotype of said test nematodes in step (b) is modified by (i) a decreased length of defecation cycle; (ii) an increased rate of germline development relative to rate of soma development; (iii) an increased rate of embryonic development; or (iv) an increased rate of post-embryonic development.

9. An isolated nucleic acid comprising:
(a) the nucleotide sequence of SEQ ID NO:1 or SEQ ID NO:7;
(b) a nucleotide sequence that encodes the amino acid sequence of SEQ ID NO:2 or SEQ ID NO:8
(c) a nucleotide sequence that is at least 80% identical to the nucleotide sequence of SEQ ID NO:1 or SEQ ID NO:7;
(d) a nucleotide sequence that is a complement of (a), (b) or (c), with the proviso that the nucleic acid does not comprise genomic sequences that are contiguous to SEQ ID NO:1 or 7, or that are contiguous to a subsequence of SEQ ID NO:1 or 7.

10. An isolated nucleic acid that hybridizes under stringent conditions to:
(a) a nucleic acid probe that consists of the nucleotide sequence of SEQ ID NO:1 or SEQ ID NO:7; or
(b) a complement of (a);
with the proviso that the isolated nucleic acid is not the nucleic acid of one of the clones designated yk357a6, K02D7, H06H21, or Y17G9.

11. The nucleic acid of claim 10, wherein the nucleic acid is a double-stranded RNA molecule.

12. The nucleic acid of claim 10 wherein the nucleic acid comprises the nucleotide sequence of SEQ ID NO:1 except that the nucleotide residue at nucleotide 354 is a thymine residue and the nucleotide at position 605 is an adenine residue.

13. The nucleic acid of claim 10 wherein the nucleic acid comprises a nucleotide sequence that encodes a polypeptide consisting of a fragment of the amino acid sequence of SEQ ID NO:2 or SEQ ID NO:8, wherein said polypeptide displays one or more functional activities of the DSC-3 or DSC-4 polypeptide.

14. An isolated polypeptide comprising SEQ ID NO:2 or SEQ ID NO:8.

15. A fragment of a polypeptide comprising at least 8 consecutive amino acids of the amino acid sequence of SEQ ID NO:2 or SEQ ID NO:8.

16. A polypeptide comprising an amino acid sequence that has at least 60% identity to the amino acid sequence of SEQ ID NO:2 or SEQ ID NO:8.

17. The polypeptide of claim 14 wherein the polypeptide comprises the amino acid sequence of SEQ ID NO:2 except that the amino acid residue at position 62 is a phenylalanine residue and the amino acid residue at position 146 is a threonine residue.

18. An antibody that immunospecifically binds to a polypeptide comprising the amino acid sequence of SEQ ID NO:2 or SEQ ID NO:8, or a fragment thereof.

19. An expression vector comprising the nucleic acid of claim 9.

20. A cell comprising a recombinant nucleic acid comprising the nucleic acid of claim 9.

21. A transgenic non-human animal comprising a transgene that comprises the nucleic acid of claim 9 or 10.

22. The transgenic non-human animal of claim 21, which is a C. elegans nematode.

23. A method for making a polypeptide comprising the steps of:
(a) culturing a cell comprising a recombinant MOLL nucleic acid under conditions that allow a MOLL polypeptide to be expressed by said cell; and
(b) isolating the MOLL polypeptide.

24. A method for identifying a compound that binds a MOLL polypeptide comprising the steps of:
(a) contacting a MOLL polypeptide with a test compound under conditions that allow said test compound to bind said MOLL polypeptide; and
(b) detecting binding of said MOLL polypeptide to said test compound.

25. A method for preventing or treating atherosclerosis or a dyslipidemia disorder in humans, said method comprising administering to a human in need thereof an amount of a pharmaceutical composition comprising an agonist or an antagonist of a MOLL polypeptide.

26. A method for preventing or treating atherosclerosis in humans, said method comprising administering to a human in need thereof an amount of a pharmaceutical composition comprising an clk-1 antagonist.

27. The method of claim 23 wherein said MOLL nucleic acid is dsc-3 or dsc-4.

28. The method of claim 24 or 25 wherein said MOLL polypeptide is DSC-3 or DSC-4.

29. The method of claim 1, 2, 3, or 6, wherein said lipid is cholesterol.

30. The method of claim 1, 2, 3, or 6, wherein said lipoprotein is LDL-like lipoprotein.

31. The method of claim 1, 2, 3, or 6, wherein a modified phenotype is detected by (i) visual inspection of the phenotype; (ii) the expression profile of one or more indicator genes; (iii) the expression of one or more reporter genes; (iv) a change in the level and/or distribution of a lipid or lipoprotein in the test nematodes.
